SEQUENCE LISTING (75 sequences)

SEQ ID NO: 1 - DNA, 21 bp, synthetic construct
  misc_feature 1..21: note = Synthetic Polynucleotide
  source 1..21: mol_type = other DNA
gccgcagcctataacaacttt

SEQ ID NO: 2 - DNA, 21 bp, synthetic construct
  misc_feature 1..21: note = Synthetic Polynucleotide
  source 1..21: mol_type = other DNA
aaagttgttataggctgcggc

SEQ ID NO: 3 - DNA, 21 bp, synthetic construct
  misc_feature 1..21: note = Synthetic Polynucleotide
  source 1..21: mol_type = other DNA
gccgcagcctataacaacttt

SEQ ID NO: 4 - DNA, 21 bp, synthetic construct
  misc_feature 1..21: note = Synthetic Polynucleotide
  source 1..21: mol_type = other DNA
ccctaattctacagaagagat

SEQ ID NO: 5 - DNA, 21 bp, synthetic construct
  misc_feature 1..21: note = Synthetic Polynucleotide
  source 1..21: mol_type = other DNA
atctcttctgtagaattaggg

SEQ ID NO: 6 - DNA, 21 bp, synthetic construct
  misc_feature 1..21: note = Synthetic Polynucleotide
  source 1..21: mol_type = other DNA
ccctaattctacagaagagat

SEQ ID NO: 7 - DNA, 21 bp, synthetic construct
  misc_feature 1..21: note = Synthetic Polynucleotide
  source 1..21: mol_type = other DNA
gatgatagaaatagggacaaa

SEQ ID NO: 8 - DNA, 21 bp, synthetic construct
  misc_feature 1..21: note = Synthetic Polynucleotide
  source 1..21: mol_type = other DNA
tttgtccctatttctatcatc

SEQ ID NO: 9 - DNA, 21 bp, synthetic construct
  misc_feature 1..21: note = Synthetic Polynucleotide
  source 1..21: mol_type = other DNA
gatgatagaaatagggacaaa

SEQ ID NO: 10 - DNA, 21 bp, synthetic construct
  misc_feature 1..21: note = Synthetic Polynucleotide
  source 1..21: mol_type = other DNA
gccacggtgaataattcagtt

SEQ ID NO: 11 - DNA, 21 bp, synthetic construct
  misc_feature 1..21: note = Synthetic Polynucleotide
  source 1..21: mol_type = other DNA
aactgaattattcaccgtggc

SEQ ID NO: 12 - DNA, 21 bp, synthetic construct
  misc_feature 1..21: note = Synthetic Polynucleotide
  source 1..21: mol_type = other DNA
gccacggtgaataattcagtt

SEQ ID NO: 13 - DNA, 21 bp, synthetic construct
  misc_feature 1..21: note = Synthetic Polynucleotide
  source 1..21: mol_type = other DNA
gcttactcattgtatgaacat

SEQ ID NO: 14 - DNA, 21 bp, synthetic construct
  misc_feature 1..21: note = Synthetic Polynucleotide
  source 1..21: mol_type = other DNA
atgttcatacaatgagtaagc

SEQ ID NO: 15 - DNA, 21 bp, synthetic construct
  misc_feature 1..21: note = Synthetic Polynucleotide
  source 1..21: mol_type = other DNA
gcttactcattgtatgaacat

SEQ ID NO: 16 - DNA, 21 bp, synthetic construct
  misc_feature 1..21: note = Synthetic Polynucleotide
  source 1..21: mol_type = other DNA
cgcgacaaacaccttctttaa

SEQ ID NO: 17 - DNA, 21 bp, synthetic construct
  misc_feature 1..21: note = Synthetic Polynucleotide
  source 1..21: mol_type = other DNA
ttaaagaaggtgtttgtcgcg

SEQ ID NO: 18 - DNA, 21 bp, synthetic construct
  misc_feature 1..21: note = Synthetic Polynucleotide
  source 1..21: mol_type = other DNA
cgcgacaaacaccttctttaa

SEQ ID NO: 19 - DNA, 21 bp, synthetic construct
  misc_feature 1..21: note = Synthetic Polynucleotide
  source 1..21: mol_type = other DNA
ctacctcagcaagacgttatt

SEQ ID NO: 20 - DNA, 21 bp, synthetic construct
  misc_feature 1..21: note = Synthetic Polynucleotide
  source 1..21: mol_type = other DNA
aataacgtcttgctgaggtag

SEQ ID NO: 21 - DNA, 21 bp, synthetic construct
  misc_feature 1..21: note = Synthetic Polynucleotide
  source 1..21: mol_type = other DNA
ctacctcagcaagacgttatt

SEQ ID NO: 22 - DNA, 21 bp, synthetic construct
  misc_feature 1..21: note = Synthetic Polynucleotide
  source 1..21: mol_type = other DNA
accaattacatgtggaataat

SEQ ID NO: 23 - DNA, 20 bp, synthetic construct
  misc_feature 1..20: note = Synthetic Polynucleotide
  source 1..20: mol_type = other DNA
attattccacatgtaattgg

SEQ ID NO: 24 - DNA, 21 bp, synthetic construct
  misc_feature 1..21: note = Synthetic Polynucleotide
  source 1..21: mol_type = other DNA
accaattacatgtggaataat

SEQ ID NO: 25 - DNA, 21 bp, synthetic construct
  misc_feature 1..21: note = Synthetic Polynucleotide
  source 1..21: mol_type = other DNA
atgaccaaacagccaagattt

SEQ ID NO: 26 - DNA, 20 bp, synthetic construct
  misc_feature 1..20: note = Synthetic Polynucleotide
  source 1..20: mol_type = other DNA
aaatcttggctgtttggtca

SEQ ID NO: 27 - DNA, 22 bp, synthetic construct
  misc_feature 1..22: note = Synthetic Polynucleotide
  source 1..22: mol_type = other DNA
aatgaccaaacagccaagattt

SEQ ID NO: 28 - DNA, 21 bp, synthetic construct
  misc_feature 1..21: note = Synthetic Polynucleotide
  source 1..21: mol_type = other DNA
gtcgttgtgtcccttcatatt

SEQ ID NO: 29 - DNA, 21 bp, synthetic construct
  misc_feature 1..21: note = Synthetic Polynucleotide
  source 1..21: mol_type = other DNA
aatatgaagggacacaacgac

SEQ ID NO: 30 - DNA, 21 bp, synthetic construct
  misc_feature 1..21: note = Synthetic Polynucleotide
  source 1..21: mol_type = other DNA
gtcgttgtgtcccttcatatt

SEQ ID NO: 31 - DNA, 21 bp, synthetic construct
  misc_feature 1..21: note = Synthetic Polynucleotide
  source 1..21: mol_type = other DNA
acaacggcatccagcgatttc

SEQ ID NO: 32 - DNA, 20 bp, synthetic construct
  misc_feature 1..20: note = Synthetic Polynucleotide
  source 1..20: mol_type = other DNA
gaaatcgctggatgccgttg

SEQ ID NO: 33 - DNA, 21 bp, synthetic construct
  misc_feature 1..21: note = Synthetic Polynucleotide
  source 1..21: mol_type = other DNA
acaacggcatccagcgatttc

SEQ ID NO: 34 - DNA, 21 bp, synthetic construct
  misc_feature 1..21: note = Synthetic Polynucleotide
  source 1..21: mol_type = other DNA
ggacaccctgcaagatgtttg

SEQ ID NO: 35 - DNA, 21 bp, synthetic construct
  misc_feature 1..21: note = Synthetic Polynucleotide
  source 1..21: mol_type = other DNA
caaacatcttgcagggtgtcc

SEQ ID NO: 36 - DNA, 21 bp, synthetic construct
  misc_feature 1..21: note = Synthetic Polynucleotide
  source 1..21: mol_type = other DNA
ggacaccctgcaagatgtttg

SEQ ID NO: 37 - DNA, 21 bp, synthetic construct
  misc_feature 1..21: note = Synthetic Polynucleotide
  source 1..21: mol_type = other DNA
caccgtgtgggctgagtttaa

SEQ ID NO: 38 - DNA, 21 bp, synthetic construct
  misc_feature 1..21: note = Synthetic Polynucleotide
  source 1..21: mol_type = other DNA
ttaaactcagcccacacggtg

SEQ ID NO: 39 - DNA, 21 bp, synthetic construct
  misc_feature 1..21: note = Synthetic Polynucleotide
  source 1..21: mol_type = other DNA
caccgtgtgggctgagtttaa

SEQ ID NO: 40 - DNA, 21 bp, synthetic construct
  misc_feature 1..21: note = Synthetic Polynucleotide
  source 1..21: mol_type = other DNA
ctttaagactttcgctatttc

SEQ ID NO: 41 - DNA, 21 bp, synthetic construct
  misc_feature 1..21: note = Synthetic Polynucleotide
  source 1..21: mol_type = other DNA
gaaatagcgaaagtcttaaag

SEQ ID NO: 42 - DNA, 22 bp, synthetic construct
  misc_feature 1..22: note = Synthetic Polynucleotide
  source 1..22: mol_type = other DNA
ctttaagactttcgctatttcg

SEQ ID NO: 43 - DNA, 21 bp, synthetic construct
  misc_feature 1..21: note = Synthetic Polynucleotide
  source 1..21: mol_type = other DNA
tcacaggcaacgagctctatg

SEQ ID NO: 44 - DNA, 21 bp, synthetic construct
  misc_feature 1..21: note = Synthetic Polynucleotide
  source 1..21: mol_type = other DNA
catagagctcgttgcctgtga

SEQ ID NO: 45 - DNA, 21 bp, synthetic construct
  misc_feature 1..21: note = Synthetic Polynucleotide
  source 1..21: mol_type = other DNA
tcacaggcaacgagctctatg

SEQ ID NO: 46 - RNA, 155 nt, synthetic construct
  misc_difference 1..155: note = Synthetic Polynucleotide
  misc_difference 57..78: note = n is a, c, g, u, or absent
  misc_difference 98..119: note = n is a, c, g, u, or absent
  source 1..155: mol_type = other RNA
cttcaggttaacccaacagaaggctaaagaaggtatattgctgttgacagtgagcgnnnnnnnnnnnnnnnnnnnnnnctgtgaagccacagatgggnnnnnnnnnnnnnnnnnnnnnntgcctactgcctcggacttcaaggggctactttagg

SEQ ID NO: 47 - RNA, 153 nt, synthetic construct
  misc_difference 1..153: note = Synthetic Polynucleotide
  misc_difference 56..77: note = n is a, c, g, or u
  misc_difference 97..118: note = n is a, c, g, or u
  source 1..153: mol_type = other RNA
gacttcttaacccaacagaaggctcgagaaggtatattgctgttgacagtgagcgnnnnnnnnnnnnnnnnnnnnnntagtgaagccacagatgtannnnnnnnnnnnnnnnnnnnnntgcctactgcctcggacttcaaggggctagaattc

SEQ ID NO: 48 - protein, 745 aa, synthetic construct
  REGION 1..745: note = Synthetic Polypeptide
MAADGYLPDWLEDTLSEGIRQWWKLKPGPPPPKAAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLAISDQTKHARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 49 - protein, 10 aa, synthetic construct
  REGION 1..10: note = Synthetic Polypeptide
LAISDQTKHA

SEQ ID NO: 50 - DNA, 1374 bp, synthetic construct
  misc_feature 1..1374: note = Synthetic Polynucleotide
  source 1..1374: mol_type = other DNA
atggtttcttactgggacaccggcgtgctgctgtgtgccctgctttcttgtctgctgctgaccggctctagcagcggctctgataccggcagacccttcgtggaaatgtacagcgagatccccgagatcatccacatgaccgagggcagagagctggtcatcccttgcagagtgacaagccccaacatcaccgtgactctgaagaagttccctctggacacactgatccccgacggcaagagaatcatctgggacagccggaagggcttcatcatcagcaacgccacctacaaagagatcggcctgctgacctgtgaagccaccgtgaatggccacctgtacaagaccaactacctgacacacagacagaccaacaccatcatcgacgtggtgctgagccctagccacggcattgaactgtctgtgggcgagaagctggtgctgaactgtaccgccagaaccgagctgaacgtgggcatcgacttcaactgggagtaccccagcagcaagcaccagcacaagaaactggtcaaccgggacctgaaaacccagagcggcagcgagatgaagaaattcctgagcaccctgaccatcgacggcgtgaccagaagtgaccagggcctgtacacatgtgccgccagctctggcctgatgaccaagaaaaacagcaccttcgtgcgggtgcacgagaaggacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaatagcacctacagagtggtgtccgtgctgaccgtgctgcaccaggattggctgaacggcaaagagtacaagtgcaaggtgtccaacaaggccctgcctgctcctatcgagaaaaccatctccaaggccaagggccagcctagggaaccccaggtttacacactgcctccaagcagggacgagctgacaaagaaccaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgctccgtgatgcacgaggccctgcacaaccactacacccagaagtccctgagcctgtctcctggcaaa

SEQ ID NO: 51 - protein, 458 aa, synthetic construct
  REGION 1..458: note = Synthetic Polypeptide
MVSYWDTGVLLCALLSCLLLTGSSSGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 5 aa, synthetic construct
  REGION 1..5: note = Synthetic Polypeptide
GYYMH

SEQ ID NO: 53 - protein, 17 aa, synthetic construct
  REGION 1..17: note = Synthetic Polypeptide
WINPNSGGTNYAQKFQG

SEQ ID NO: 54 - protein, 20 aa, synthetic construct
  REGION 1..20: note = Synthetic Polypeptide
SPNPYYYDSSGYYYPGAFDI

SEQ ID NO: 55 - protein, 11 aa, synthetic construct
  REGION 1..11: note = Synthetic Polypeptide
GGNNIGSKSVH

SEQ ID NO: 56 - protein, 7 aa, synthetic construct
  REGION 1..7: note = Synthetic Polypeptide
DDSDRPS

SEQ ID NO: 57 - protein, 11 aa, synthetic construct
  REGION 1..11: note = Synthetic Polypeptide
QVWDSSSDHWV

SEQ ID NO: 58 - DNA, 1686 bp, synthetic construct
  misc_feature 1..1686: note = Synthetic Polynucleotide
  source 1..1686: mol_type = other DNA
atgcaaagaggcgccgctctctgtctgagactgtggctgtgtctgggcctgctggatggactggtgtctggctacagcatgacccctccaacactgaacatcaccgaggaatcccacgtgatcgacaccggcgatagcctgagcatcagctgcagaggacagcaccctctggaatgggcttggcctggtgctcaagaagctcctgccacaggcgacaaggacagcgaggatacaggcgttgtgcgggattgcgagggcacagatgccagaccttactgcaaggtgctgctgctgcacgaagtgcacgcccaggataccggcagctacgtgtgctactacaagtacatcaaggcccggatcgagggcaccacagccgcaagctcttatgtgttcgtgcgggacttcgagcagcccttcatcaacaagcccgacacactgctggtcaaccggaaggacgctatgtgggtgccctgtctggtgtctatccccggcctgaatgtgaccctgagaagccagagttccgtgctgtggcctgatggccaagaggtcgtgtgggacgatagaaggggcatgctggtgtccacacctctgctgcatgatgccctgtacctgcagtgcgagacaacctggggcgaccaggacttcctgagcaaccctttcctggtgcacatcaccggcaacgagctgtacgacatccagctgctgcctcgcaagagcctggaactgctcgtgggagagaagctggtgctgaactgtaccgtgtgggccgagttcaatagcggcgtgaccttcgactgggactaccctggaaagcaggccgagcgtggaaaatgggtgcccgagagaagaagccagcagacccacacagagctgagcagcatcctgaccatccacaacgtgtcccagcacgatctgggctcttacgtgtgcaaggccaacaacggcatccagcggttccgggaaagcaccgaagtgatcgtgcatgaggaacccaagagctgcgacaagacacacacctgtcctccatgtcctgctccagagcttctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaatgccaagaccaagcctagagaggaacagtacaacagcacctacagagtggtgtccgtgctgaccgtgctgcatcaggattggctgaacggcaaagagtacaagtgcaaggtgtccaacaaggccctgcctgctcctatcgagaaaaccatctccaaggccaagggccagcctcgggaacctcaagtgtataccctgcctcctagccgcgacgaactcaccaagaatcaagtgtctctgacatgtctcgtgaaggggttttaccccagcgacattgccgtcgagtgggagtccaatggacaacccgagaacaattataagaccacgccaccagtcctggactccgacggctcattttttctctactccaaactgaccgtggataagtcccggtggcagcaagggaatgtgttttcctgtagcgtgatgcatgaagctctccacaatcattacacccaaaaatctctgtctctgagccccggcaaatga

SEQ ID NO: 59 - protein, 1674 aa, synthetic construct
  REGION 1..1674: note = Synthetic Polypeptide
ATGCAGAGGGGAGCCGCCCTGTGCCTGAGGCTGTGGCTGTGCCTGGGCCTGCTGGACGGCCTGGTGTCTGGCTACAGCATGACCCCCCCTACACTGAACATCACCGAGGAGAGCCACGTGATCGACACAGGCGATAGCCTGTCCATCTCTTGCAGGGGCCAGCACCCCCTGGAGTGGGCATGGCCTGGAGCACAGGAGGCACCAGCCACCGGCGACAAGGATAGCGAGGACACAGGAGTGGTGCGGGACTGCGAGGGAACCGATGCCAGACCTTACTGTAAGGTGCTGCTGCTGCACGAGGTGCACGCCCAGGATACAGGCTCCTACGTGTGCTACTATAAGTATATCAAGGCAAGGATCGAGGGAACCACAGCAGCCAGCTCCTACGTGTTCGTGCGGGATTTTGAGCAGCCTTTCATCAACAAGCCAGACACCCTGCTGGTGAATCGGAAGGATGCCATGTGGGTGCCCTGTCTGGTGTCTATCCCTGGCCTGAATGTGACACTGAGAAGCCAGTCTAGCGTGCTGTGGCCAGACGGACAGGAGGTGGTGTGGGACGATCGGAGAGGCATGCTGGTGAGCACCCCTCTGCTGCACGATGCCCTGTACCTGCAGTGCGAGACAACATGGGGCGACCAGGATTTTCTGTCCAACCCTTTCCTGGTGCACATCACAGGCAATGAGCTGTATGACATCCAGCTGCTGCCACGGAAGTCCCTGGAGCTGCTGGTGGGCGAGAAGCTGGTGCTGAACTGTACCGTGTGGGCCGAGTTTAATTCTGGCGTGACATTCGACTGGGATTACCCCGGCAAGCAGGCCGAGAGGGGCAAGTGGGTGCCTGAGAGGCGCAGCCAGCAGACCCACACAGAGCTGTCCTCTATCCTGACCATCCACAACGTGAGCCAGCACGATCTGGGCTCCTACGTGTGCAAGGCCAACAATGGCATCCAGCGGTTTAGAGAGTCTACAGAAGTGATCGTGCACGAGGAGAGGAAGTGCTGCGTGGAGTGCCCACCATGTCCAGCACCTCCAGTGGCAGGACCATCCGTGTTCCTGTTTCCACCTAAGCCTAAGGACACCCTGATGATCAGCCGCACCCCAGAGGTGACATGCGTGGTGGTGGACGTGTCCCACGAGGATCCAGAGGTGCAGTTCAACTGGTACGTGGATGGCGTGGAGGTGCACAATGCCAAGACCAAGCCCAGGGAGGAGCAGTTTAATTCTACCTTCCGCGTGGTGAGCGTGCTGACAGTGGTGCACCAGGACTGGCTGAACGGCAAGGAGTATAAGTGCAAGGTGTCTAATAAGGGCCTGCCCGCCCCTATCGAGAAGACCATCAGCAAGACAAAGGGACAGCCACGGGAGCCACAGGTGTACACCCTGCCACCATCCAGAGAGGAGATGACCAAGAACCAGGTGTCTCTGACATGTCTGGTGAAGGGCTTTTATCCAAGCGACATCGCCGTGGAGTGGGAGTCCAATGGCCAGCCCGAGAACAATTACAAGACCACACCTCCAATGCTGGACTCCGATGGCTCTTTCTTTCTGTATTCCAAGCTGACCGTGGATAAGTCTCGGTGGCAGCAGGGCAACGTGTTCAGCTGTTCCGTGATGCACGAGGCCCTGCACAATCACTACACACAGAAGTCTCTGAGCCTGTCCCCCGGCAAGTGA

SEQ ID NO: 60 - DNA, 1257 bp, synthetic construct
  misc_feature 1..1257: note = Synthetic Polynucleotide
  source 1..1257: mol_type = other DNA
atgcaagctctggtgctgctgctgtgtatcggagccctgctgggccacagctcctgtcaaaatcctgcctctccacctgaggaaggcagccccgatccagattctacaggcgccctggtggaagaagaggacccattcttcaaggtgcccgtgaacaaactggccgctgccgtgtccaacttcggctacgacctgtacagagtgcggagcagcacaagccccaccaccaatgttctgctgagccctctgtctgtggccaccgctctttctgctctgtctctgggagccgagcagagaaccgagagcatcattcacagagccctgtactacgatctgatcagcagccctgacatccacggcacctacaaagaactgctggacaccgtgacagcccctcagaagaatctgaagtccgccagccggatcgtgttcgagaagaagctgcggatcaagagcagcttcgtggcccctctggaaaagagctacggcaccagacctagagtgctgaccggcaatcccagactggacctgcaagagatcaacaactgggtgcaagcccagatgaagggcaagctggccagaagcaccaaagagatccccgacgagatcagcatcctgctgctgggcgtcgcccactttaaaggccagtgggtcaccaagttcgactccagaaagaccagcctcgaggacttctacctggacgaggaacggaccgtcagagtgcccatgatgagcgatcctaaggccgtgctgagatacggcctggatagcgacctgagctgcaagattgctcagctgcctctgaccggctctatgagcatcatattctttctgcccctgaaagtgacccagaatctgaccctgatcgaggaaagcctgaccagcgagttcatccacgacatcgaccgcgagctgaaaaccgtgcaggctgtgctgactgtgcccaagctgaagctgagctacgagggcgaagtgaccaagagcctgcaagaaatgaagctgcagagcctgttcgacagccccgacttcagcaagatcaccggcaagcccatcaagctgacccaggtggaacacagagccggcttcgagtggaatgaagatggcgccggaaccacaccttctccaggactgcaacctgctcacctgacctttccactggactaccacctgaaccagcctttcatcttcgtgctgcgggacacagatactggcgccctgctgttcatcggcaagatcctggatcctagaggcccctga

SEQ ID NO: 61 - DNA, 145 bp, Adeno-associated virus 2
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 62 - DNA, 145 bp, Adeno-associated virus 2
  source 1..145: mol_type = other DNA
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 63 - DNA, 245 bp, synthetic construct
  misc_feature 1..245: note = Synthetic Polynucleotide
  source 1..245: mol_type = other DNA
ggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatca

SEQ ID NO: 64 - DNA, 4297 bp, synthetic construct
  misc_feature 1..4297: note = Synthetic Polynucleotide
  source 1..4297: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctatcgattgaattccaggcgcgccatcctgcaggtattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgtacaggatatcttgctagcacgccaccatggtttcttactgggacaccggcgtgctgctgtgtgccctgctttcttgtctgctgctgaccggctctagcagcggctctgataccggcagacccttcgtggaaatgtacagcgagatccccgagatcatccacatgaccgagggcagagagctggtcatcccttgcagagtgacaagccccaacatcaccgtgactctgaagaagttccctctggacacactgatccccgacggcaagagaatcatctgggacagccggaagggcttcatcatcagcaacgccacctacaaagagatcggcctgctgacctgtgaagccaccgtgaatggccacctgtacaagaccaactacctgacacacagacagaccaacaccatcatcgacgtggtgctgagccctagccacggcattgaactgtctgtgggcgagaagctggtgctgaactgtaccgccagaaccgagctgaacgtgggcatcgacttcaactgggagtaccccagcagcaagcaccagcacaagaaactggtcaaccgggacctgaaaacccagagcggcagcgagatgaagaaattcctgagcaccctgaccatcgacggcgtgaccagaagtgaccagggcctgtacacatgtgccgccagctctggcctgatgaccaagaaaaacagcaccttcgtgcgggtgcacgagaaggacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaatagcacctacagagtggtgtccgtgctgaccgtgctgcaccaggattggctgaacggcaaagagtacaagtgcaaggtgtccaacaaggccctgcctgctcctatcgagaaaaccatctccaaggccaagggccagcctagggaaccccaggtttacacactgcctccaagcagggacgagctgacaaagaaccaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgctccgtgatgcacgaggccctgcacaaccactacacccagaagtccctgagcctgtctcctggcaaacggaagagaagaggcagcggcgaaggcagaggatccctgcttacatgtggcgacgtggaagagaaccccggacctatgcaagctctggtgctgctgctgtgtatcggagccctgctgggccacagctcctgtcaaaatcctgcctctccacctgaggaaggcagccccgatccagattctacaggcgccctggtggaagaagaggacccattcttcaaggtgcccgtgaacaaactggccgctgccgtgtccaacttcggctacgacctgtacagagtgcggagcagcacaagccccaccaccaatgttctgctgagccctctgtctgtggccaccgctctttctgctctgtctctgggagccgagcagagaaccgagagcatcattcacagagccctgtactacgatctgatcagcagccctgacatccacggcacctacaaagaactgctggacaccgtgacagcccctcagaagaatctgaagtccgccagccggatcgtgttcgagaagaagctgcggatcaagagcagcttcgtggcccctctggaaaagagctacggcaccagacctagagtgctgaccggcaatcccagactggacctgcaagagatcaacaactgggtgcaagcccagatgaagggcaagctggccagaagcaccaaagagatccccgacgagatcagcatcctgctgctgggcgtcgcccactttaaaggccagtgggtcaccaagttcgactccagaaagaccagcctcgaggacttctacctggacgaggaacggaccgtcagagtgcccatgatgagcgatcctaaggccgtgctgagatacggcctggatagcgacctgagctgcaagattgctcagctgcctctgaccggctctatgagcatcatattctttctgcccctgaaagtgacccagaatctgaccctgatcgaggaaagcctgaccagcgagttcatccacgacatcgaccgcgagctgaaaaccgtgcaggctgtgctgactgtgcccaagctgaagctgagctacgagggcgaagtgaccaagagcctgcaagaaatgaagctgcagagcctgttcgacagccccgacttcagcaagatcaccggcaagcccatcaagctgacccaggtggaacacagagccggcttcgagtggaatgaagatggcgccggaaccacaccttctccaggactgcaacctgctcacctgacctttccactggactaccacctgaaccagcctttcatcttcgtgctgcgggacacagatactggcgccctgctgttcatcggcaagatcctggatcctagaggcccctgagccacgcgtaacacgtgcatgcgagagatctgcggccgcgagctcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcaatgcatcctagccggaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 65 - DNA, 4726 bp, synthetic construct
  misc_feature 1..4726: note = Synthetic Polynucleotide
  source 1..4726: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctatcgattgaattccaggcgcgccatcctgcaggtattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgtacaggatatcttgctagcacgccaccatggtttcttactgggacaccggcgtgctgctgtgtgccctgctttcttgtctgctgctgaccggctctagcagcggctctgataccggcagacccttcgtggaaatgtacagcgagatccccgagatcatccacatgaccgagggcagagagctggtcatcccttgcagagtgacaagccccaacatcaccgtgactctgaagaagttccctctggacacactgatccccgacggcaagagaatcatctgggacagccggaagggcttcatcatcagcaacgccacctacaaagagatcggcctgctgacctgtgaagccaccgtgaatggccacctgtacaagaccaactacctgacacacagacagaccaacaccatcatcgacgtggtgctgagccctagccacggcattgaactgtctgtgggcgagaagctggtgctgaactgtaccgccagaaccgagctgaacgtgggcatcgacttcaactgggagtaccccagcagcaagcaccagcacaagaaactggtcaaccgggacctgaaaacccagagcggcagcgagatgaagaaattcctgagcaccctgaccatcgacggcgtgaccagaagtgaccagggcctgtacacatgtgccgccagctctggcctgatgaccaagaaaaacagcaccttcgtgcgggtgcacgagaaggacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaatagcacctacagagtggtgtccgtgctgaccgtgctgcaccaggattggctgaacggcaaagagtacaagtgcaaggtgtccaacaaggccctgcctgctcctatcgagaaaaccatctccaaggccaagggccagcctagggaaccccaggtttacacactgcctccaagcagggacgagctgacaaagaaccaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgctccgtgatgcacgaggccctgcacaaccactacacccagaagtccctgagcctgtctcctggcaaacggaagagaagaggcagcggcgaaggcagaggatccctgcttacatgtggcgacgtggaagagaaccccggacctatgcaaagaggcgccgctctctgtctgagactgtggctgtgtctgggcctgctggatggactggtgtctggctacagcatgacccctccaacactgaacatcaccgaggaatcccacgtgatcgacaccggcgatagcctgagcatcagctgcagaggacagcaccctctggaatgggcttggcctggtgctcaagaagctcctgccacaggcgacaaggacagcgaggatacaggcgttgtgcgggattgcgagggcacagatgccagaccttactgcaaggtgctgctgctgcacgaagtgcacgcccaggataccggcagctacgtgtgctactacaagtacatcaaggcccggatcgagggcaccacagccgctagctcttatgtgttcgtgcgggacttcgagcagcccttcatcaacaagcccgacacactgctggtcaaccggaaggacgctatgtgggtgccctgtctggtgtctatccccggcctgaatgtgaccctgagaagccagagttccgtgctgtggcctgatggccaagaggtcgtgtgggacgatagaaggggcatgctggtgtccacacctctgctgcatgatgccctgtacctgcagtgcgagacaacctggggcgaccaggacttcctgagcaaccctttcctggtgcacatcaccggcaacgagctgtacgacatccagctgctgcctcgcaagagcctggaactgctcgtgggagagaagctggtgctgaactgtaccgtgtgggccgagttcaatagcggcgtgaccttcgactgggactaccctggaaagcaggccgagcgtggaaaatgggtgcccgagagaagaagccagcagacccacacagagctgagcagcatcctgaccatccacaacgtgtcccagcacgatctgggctcttacgtgtgcaaggccaacaacggcatccagcggttccgggaaagcaccgaagtgatcgtgcatgaggaacccaagagctgcgacaagacacacacctgtcctccatgtcctgctccagagcttctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaatgccaagaccaagcctagagaggaacagtacaacagcacctacagagtggtgtccgtgctgaccgtgctgcatcaggattggctgaacggcaaagagtacaagtgcaaggtgtccaacaaggccctgcctgctcctatcgagaaaaccatctccaaggccaagggccagcctcgggaacctcaagtgtataccctgcctcctagccgcgacgaactcaccaagaatcaagtgtctctgacatgtctcgtgaaggggttttaccccagcgacattgccgtcgagtgggagtccaatggacaacccgagaacaattataagaccacgccaccagtcctggactccgacggctcattttttctctactccaaactgaccgtggataagtcccggtggcagcaagggaatgtgttttcctgtagcgtgatgcatgaagctctccacaatcattacacccaaaaatctctgtctctgagccccggcaaatgagccacgcgtaacacgtgcatgcgagagatctgcggccgcgagctcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcaatgcatcctagccggaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 66 - DNA, 4642 bp, synthetic construct
  misc_feature 1..4642: note = Synthetic Polynucleotide
  source 1..4642: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctatcgattgaattccaggcgcgccatcctgcaggtattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgtacaggatatcttgctagcacgccaccatggtttcttactgggacaccggcgtgctgctgtgtgccctgctttcttgtctgctgctgaccggctctagcagcggctctgataccggcagacccttcgtggaaatgtacagcgagatccccgagatcatccacatgaccgagggcagagagctggtcatcccttgcagagtgacaagccccaacatcaccgtgactctgaagaagttccctctggacacactgatccccgacggcaagagaatcatctgggacagccggaagggcttcatcatcagcaacgccacctacaaagagatcggcctgctgacctgtgaagccaccgtgaatggccacctgtacaagaccaactacctgacacacagacagaccaacaccatcatcgacgtggtgctgagccctagccacggcattgaactgtctgtgggcgagaagctggtgctgaactgtaccgccagaaccgagctgaacgtgggcatcgacttcaactgggagtaccccagcagcaagcaccagcacaagaaactggtcaaccgggacctgaaaacccagagcggcagcgagatgaagaaattcctgagcaccctgaccatcgacggcgtgaccagaagtgaccagggcctgtacacatgtgccgccagctctggcctgatgaccaagaaaaacagcaccttcgtgcgggtgcacgagaaggacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaatagcacctacagagtggtgtccgtgctgaccgtgctgcaccaggattggctgaacggcaaagagtacaagtgcaaggtgtccaacaaggccctgcctgctcctatcgagaaaaccatctccaaggccaagggccagcctagggaaccccaggtttacacactgcctccaagcagggacgagctgacaaagaaccaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgctccgtgatgcacgaggccctgcacaaccactacacccagaagtccctgagcctgtctcctggcaaacggaagagaagaggcagcggcgaaggcagaggatccctgcttacatgtggcgacgtggaagagaaccccggacctatggattggacctggtccatcctgtttctggtggccgctgccacaggcacatactctcaggttcagctggtgcagtctggcgccgaagtgaaaaaacctggcgcctccgtgaaggtgtcctgcaaggctagcggctacacctttaccggctactacatgcactgggtccgacaggctccaggacagggacttgaatggatgggctggatcaaccccaatagcggcggcaccaattacgcccagaaattccagggcagagtgaccatgaccagagacaccagcatcagcaccgcctacatggaactgagccggctgagatccgatgacaccgccgtgtactactgcgccagatctcccaatccttactactacgacagcagcgggtactactacccaggcgcctttgatatctggggccagggcacaatggtcaccgtgtcatctgcatctgtggccgctcctagcgtgttcatcttcccaccttccgacgaacagctgaagtctggcacagccagcgtcgtgtgcctgctgaacaacttctaccccagagaagccaaggtgcagtggaaggtggacaacgctctgcagtccggcaacagccaagagagcgtgacagagcaggacagcaaggactccacctacagcctgagcagcaccctgacactgagcaaggccgactacgagaagcacaaagtgtacgcctgcgaagtgacccaccagggcctttctagccctgtgaccaagagcttcaaccggggcgagtgcgataagacacacacaggcggaagcagcggcagcggatctggatctaccggcacatctagctctggcaccggaacatctgccggcacaactggcacaagcgcctctacatctggaagcggttctggcggaggcggaggatctggtggtggtggatctgctggcggaacagctacagctggcgcttctagcggcagcagctatgtgctgacacagcctccatccgtgtctgtggcacctggacagaccgccagaattacctgtggcggcaacaacatcggcagcaagagcgtgcactggtatcagcagaagcctggacaggcaccagtgctggtggtgtacgacgacagcgatagacctagcggcatccccgagagattcagcggctctaacagcggcaataccgccacactgaccatcagcagagtggaagctggcgacgaggccgattactactgccaagtgtgggacagcagcagcgaccactgggttttcggcggaggcaccaaactgacagtgctgtctagcgccagcacaaagggcccatctgtgttccctctggctcccagcagcaagtctacaagcggaggaacagccgctctgggctgcctcgtgaaggattactttcccgagcctgtgaccgtgtcctggaatagcggagcactgacaagcggcgtgcacacctttccagctgtgctgcaaagctccggcctgtactctctgagcagcgtggtcacagtgcctagctctagcctgggcacccagacctacatctgcaatgtgaaccacaagcctagcaacaccaaggtcgacaagaaggtggaacccaagagctgctgagccacgcgtaacacgtgcatgcgagagatctgcggccgcgagctcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcaatgcatcctagccggaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 67 - DNA, 4645 bp, synthetic construct
  misc_feature 1..4645: note = Synthetic Polynucleotide
  source 1..4645: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctatcgattgaattccaggcgcgccatcctgcaggtattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgtacaggatatcttgctagcacgccaccatggtttcttactgggacaccggcgtgctgctgtgtgccctgctttcttgtctgctgctgaccggctctagcagcggctctgataccggcagacccttcgtggaaatgtacagcgagatccccgagatcatccacatgaccgagggcagagagctggtcatcccttgcagagtgacaagccccaacatcaccgtgactctgaagaagttccctctggacacactgatccccgacggcaagagaatcatctgggacagccggaagggcttcatcatcagcaacgccacctacaaagagatcggcctgctgacctgtgaagccaccgtgaatggccacctgtacaagaccaactacctgacacacagacagaccaacaccatcatcgacgtggtgctgagccctagccacggcattgaactgtctgtgggcgagaagctggtgctgaactgtaccgccagaaccgagctgaacgtgggcatcgacttcaactgggagtaccccagcagcaagcaccagcacaagaaactggtcaaccgggacctgaaaacccagagcggcagcgagatgaagaaattcctgagcaccctgaccatcgacggcgtgaccagaagtgaccagggcctgtacacatgtgccgccagctctggcctgatgaccaagaaaaacagcaccttcgtgcgggtgcacgagaaggacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaatagcacctacagagtggtgtccgtgctgaccgtgctgcaccaggattggctgaacggcaaagagtacaagtgcaaggtgtccaacaaggccctgcctgctcctatcgagaaaaccatctccaaggccaagggccagcctagggaaccccaggtttacacactgcctccaagcagggacgagctgacaaagaaccaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgctccgtgatgcacgaggccctgcacaaccactacacccagaagtccctgagcctgtctcctggcaaacggaagagaagaggcagcggcgaaggcagaggatccctgcttacatgtggcgacgtggaagagaaccccggacctatggttctgcagacccaggtgttcatcagcctgctgctgtggatctctggcgcctacggcagctatgtgctgacacagcctccatccgtgtctgtggctccaggacagaccgccagaattacctgcggcggcaacaacatcggcagcaagagcgtgcactggtatcagcagaagcctggacaggctccagtgctggtggtgtacgacgacagcgatagacctagcggcatccccgagagattcagcggcagcaattccggcaataccgccacactgaccatcagcagagtggaagctggcgacgaggccgactactactgccaagtgtgggatagcagcagcgaccactgggttttcggcggaggcaccaaactgacagtgctgagcagcgcctctacaaagggccctagtgtgttccctctggctcccagcagcaagtctacatctggcggaacagccgctctgggctgcctcgtgaaggattactttcccgagcctgtgaccgtgtcctggaatagcggagcactgacaagcggcgtgcacacctttccagctgtgctgcaaagcagcggcctgtactctctgtccagcgtggtcacagtgccaagctctagcctgggcacccagacctacatctgcaatgtgaaccacaagcctagcaacaccaaggtcgacaagaaggtggaacccaagagctgtggcggcagctctggttctggatctggcagcacaggcacatctagctctggcaccggaacaagcgctggcacaactggcacatctgccagcacaagcggatctggaagtggcggaggcggaggatctggtggcggtggatctgcaggcggaactgctacagctggcgcttctagtggaagccaggtgcagctggttcagtctggcgccgaagtgaaaaagcctggcgcctctgtgaaggtgtcctgcaaggccagcggctacacctttaccggctactacatgcactgggtccgacaggcaccaggacagggacttgaatggatgggctggatcaaccccaatagcggcggcaccaattacgcccagaaattccagggcagagtgaccatgaccagagacaccagcatcagcaccgcctacatggaactgagccggctgagatccgatgacaccgccgtgtactactgcgccagatctcccaatccttactactacgacagcagcgggtactactacccaggcgcctttgatatctggggccagggcaccatggtcaccgtgtcatctgcatctgtggccgctcctagcgtgttcatcttcccaccttccgacgaacagctgaagtccggcacagcctctgtcgtgtgcctgctgaacaacttctaccccagagaagccaaagtgcagtggaaggtggacaacgccctgcagagcggcaatagccaagagagcgtgaccgagcaggacagcaaggactctacctacagcctgagcagcaccctgacactgagcaaggccgattacgagaagcacaaggtgtacgcctgcgaagtgacacaccagggcctgtctagccctgtgaccaagagcttcaatcggggcgagtgcgacaagacccacacctaagccacgcgtaacacgtgcatgcgagagatctgcggccgcgagctcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcaatgcatcctagccggaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 68 - DNA, 3834 bp, synthetic construct
  misc_feature 1..3834: note = Synthetic Polynucleotide
  source 1..3834: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctatcgattgaattccccggggatccactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcgcgacttcttaacccaacagaaggctcgagaaggtatattgctgttgacagtgagcggcttactcattgtatgaacatttagtgaagccacagatgtaaatgttcatacaatgagtaagctgcctactgcctcggacttcaaggggctagaattcgcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagtctagagtcgacctgcaggtggatatcttggctagcacgccaccatggtttcttactgggacaccggcgtgctgctgtgtgccctgctttcttgtctgctgctgaccggctctagcagcggctctgataccggcagacccttcgtggaaatgtacagcgagatccccgagatcatccacatgaccgagggcagagagctggtcatcccttgcagagtgacaagccccaacatcaccgtgactctgaagaagttccctctggacacactgatccccgacggcaagagaatcatctgggacagccggaagggcttcatcatcagcaacgccacctacaaagagatcggcctgctgacctgtgaagccaccgtgaatggccacctgtacaagaccaactacctgacacacagacagaccaacaccatcatcgacgtggtgctgagccctagccacggcattgaactgtctgtgggcgagaagctggtgctgaactgtaccgccagaaccgagctgaacgtgggcatcgacttcaactgggagtaccccagcagcaagcaccagcacaagaaactggtcaaccgggacctgaaaacccagagcggcagcgagatgaagaaattcctgagcaccctgaccatcgacggcgtgaccagaagtgaccagggcctgtacacatgtgccgccagctctggcctgatgaccaagaaaaacagcaccttcgtgcgggtgcacgagaaggacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaatagcacctacagagtggtgtccgtgctgaccgtgctgcaccaggattggctgaacggcaaagagtacaagtgcaaggtgtccaacaaggccctgcctgctcctatcgagaaaaccatctccaaggccaagggccagcctagggaaccccaggtttacacactgcctccaagcagggacgagctgacaaagaaccaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgctccgtgatgcacgaggccctgcacaaccactacacccagaagtccctgagcctgtctcctggcaaatgagccacgcgtaacacgtgggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcaatgcatggccggccggaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 69 - DNA, 3834 bp, synthetic construct
  misc_feature 1..3834: note = Synthetic Polynucleotide
  source 1..3834: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctatcgattgaattccccggggatccactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcgcgacttcttaacccaacagaaggctcgagaaggtatattgctgttgacagtgagcggctacctcagcaagacgttatttagtgaagccacagatgtaaataacgtcttgctgaggtagctgcctactgcctcggacttcaaggggctagaattcgcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagtctagagtcgacctgcaggtggatatcttggctagcacgccaccatggtttcttactgggacaccggcgtgctgctgtgtgccctgctttcttgtctgctgctgaccggctctagcagcggctctgataccggcagacccttcgtggaaatgtacagcgagatccccgagatcatccacatgaccgagggcagagagctggtcatcccttgcagagtgacaagccccaacatcaccgtgactctgaagaagttccctctggacacactgatccccgacggcaagagaatcatctgggacagccggaagggcttcatcatcagcaacgccacctacaaagagatcggcctgctgacctgtgaagccaccgtgaatggccacctgtacaagaccaactacctgacacacagacagaccaacaccatcatcgacgtggtgctgagccctagccacggcattgaactgtctgtgggcgagaagctggtgctgaactgtaccgccagaaccgagctgaacgtgggcatcgacttcaactgggagtaccccagcagcaagcaccagcacaagaaactggtcaaccgggacctgaaaacccagagcggcagcgagatgaagaaattcctgagcaccctgaccatcgacggcgtgaccagaagtgaccagggcctgtacacatgtgccgccagctctggcctgatgaccaagaaaaacagcaccttcgtgcgggtgcacgagaaggacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaatagcacctacagagtggtgtccgtgctgaccgtgctgcaccaggattggctgaacggcaaagagtacaagtgcaaggtgtccaacaaggccctgcctgctcctatcgagaaaaccatctccaaggccaagggccagcctagggaaccccaggtttacacactgcctccaagcagggacgagctgacaaagaaccaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgctccgtgatgcacgaggccctgcacaaccactacacccagaagtccctgagcctgtctcctggcaaatgagccacgcgtaacacgtgggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcaatgcatggccggccggaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 70 - DNA, 3834 bp, synthetic construct
  misc_feature 1..3834: note = Synthetic Polynucleotide
  source 1..3834: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctatcgattgaattccccggggatccactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcgcgacttcttaacccaacagaaggctcgagaaggtatattgctgttgacagtgagcgcaccgtgtgggctgagtttaactagtgaagccacagatgtagttaaactcagcccacacggtgtgcctactgcctcggacttcaaggggctagaattcgcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagtctagagtcgacctgcaggtggatatcttggctagcacgccaccatggtttcttactgggacaccggcgtgctgctgtgtgccctgctttcttgtctgctgctgaccggctctagcagcggctctgataccggcagacccttcgtggaaatgtacagcgagatccccgagatcatccacatgaccgagggcagagagctggtcatcccttgcagagtgacaagccccaacatcaccgtgactctgaagaagttccctctggacacactgatccccgacggcaagagaatcatctgggacagccggaagggcttcatcatcagcaacgccacctacaaagagatcggcctgctgacctgtgaagccaccgtgaatggccacctgtacaagaccaactacctgacacacagacagaccaacaccatcatcgacgtggtgctgagccctagccacggcattgaactgtctgtgggcgagaagctggtgctgaactgtaccgccagaaccgagctgaacgtgggcatcgacttcaactgggagtaccccagcagcaagcaccagcacaagaaactggtcaaccgggacctgaaaacccagagcggcagcgagatgaagaaattcctgagcaccctgaccatcgacggcgtgaccagaagtgaccagggcctgtacacatgtgccgccagctctggcctgatgaccaagaaaaacagcaccttcgtgcgggtgcacgagaaggacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaatagcacctacagagtggtgtccgtgctgaccgtgctgcaccaggattggctgaacggcaaagagtacaagtgcaaggtgtccaacaaggccctgcctgctcctatcgagaaaaccatctccaaggccaagggccagcctagggaaccccaggtttacacactgcctccaagcagggacgagctgacaaagaaccaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgctccgtgatgcacgaggccctgcacaaccactacacccagaagtccctgagcctgtctcctggcaaatgagccacgcgtaacacgtgggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcaatgcatggccggccggaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 71 - protein, 8 aa, synthetic construct
  REGION 1..8: note = Synthetic Polypeptide
DYKDDDDK

SEQ ID NO: 72 - RNA, 22 nt, synthetic construct
  misc_feature 1..22: note = Reverse Transcriptase PCR primer
  source 1..22: mol_type = other RNA
aatgttcatacaatgagtaagc

SEQ ID NO: 73 - RNA, 22 nt, synthetic construct
  misc_feature 1..22: note = Reverse Transcriptase PCR Primer
  source 1..22: mol_type = other RNA
aataacgtcttgctgaggtagc

SEQ ID NO: 74 - protein, 7 aa, synthetic construct
  REGION 1..7: note = Synthetic Polypeptide
ISDQTKH

SEQ ID NO: 75 - protein, 10 aa, synthetic construct
  VARIANT 1..10: note = Synthetic Polypeptide
  VARIANT 1..2: note = misc_feature - Xaa is a A, L, G, S, T, P, or absent
  VARIANT 10: note = misc_feature - Xaa is a A, L, G, S, T, P, or absent
XXISDQTKHX

(19) United States
(12) Patent Application Publication
(10) Pub. No.: US 2024/0226336 A9
(43) Pub. Date: Jul. 11, 2024

(54) Compositions and Methods for Treatment of Ocular Disease Associated with Angiogenesis

(71) Applicant: 4D MOLECULAR THERAPEUTICS INC., Emeryville, CA (US)

(72) Inventors: Christian BURNS, Emeryville, CA (US); Melissa CALTON, Emeryville, CA (US); Meredith LEONG, Emeryville, CA (US); Paul SZYMANSKI, Emeryville, CA (US)

(73) Assignee: 4D MOLECULAR THERAPEUTICS INC., Emeryville, CA (US)

(21) Appl. No.: 18/482,628

(22) Filed: Oct. 6, 2023

Related U.S. Application Data
(63) Continuation of application No. PCT/US22/26395, filed on Apr. 26, 2022.
(60) Provisional application No. 63/180,247, filed on Apr. 27, 2021.

(51) Int. Cl. A61K 48/00; A61P 27/02; C07K 14/71; C07K 16/22; C12N 15/113; C12N 15/86

(52) U.S. Cl. CPC A61K 48/0066; A61K 48/0058; A61K 48/0075; A61P 27/02; C07K 14/71; C07K 16/22; C12N 15/1136; C12N 15/86; C07K 2317/55; C07K 2319/30; C07K 2319/32; C12N 2310/141; C12N 2750/14122; C12N 2750/14143; C12N 2750/14171

(57) ABSTRACT

The present disclosure provides compositions and methods for the treatment of ocular diseases associated with angiogenesis, particularly wet age-related macular degeneration.

### CROSS REFERENCE TO RELATED APPLICATIONS

This application is a continuation of International Application No. PCT/US2022/026395, filed Apr. 26, 2022, which claims the benefit of U.S. Provisional Patent Application Ser. No. 63/180,247, filed Apr. 27, 2021, the full disclosure of which is incorporated herein by reference.

### SEQUENCE LISTING SUBMISSION VIA EFS-WEB

A computer readable XML file, entitled “090400-5018-US-Sequence-Listing” created on or about Oct. 4, 2023, with a file size of about 132,000 bytes, contains the sequence listing for this application and is hereby incorporated by reference in its entirety.

### BACKGROUND OF THE INVENTION

The vascular endothelial growth factor (VEGF) proteins and their receptors (VEGFRs) play important roles in vasculogenesis, the development of the embryonic vasculature from early differentiating endothelial cells, angiogenesis, the process of forming new blood vessels from pre-existing ones, and lymphangiogenesis, the process of forming new lymph vessels.

Ocular vascular diseases such as age-related macular degeneration and diabetic retinopathy are due to abnormal choroidal or retinal neovascularization respectively. Since the retina consists of well-defined layers of neuronal, glial, and vascular elements, relatively small disturbances such as those seen in vascular proliferation or edema can lead to significant loss of visual function. Inherited retinal degenerations, such as Retinitis Pigmentosa, are also associated with vascular abnormalities, such as arteriolar narrowing and vascular atrophy.

Strategies have been employed to block the function of VEGF. Current standard-of-care treatments include intravitreal (IVT) injections of protein therapies, such as aflibercept, ranibizumab, and brolucizumab that bind to vascular endothelial growth factor A (VEGF-A, VEGF) to prevent binding to its receptors. Regimens of anti-VEGF therapies shown to be safe and effective require repeated monthly-to-bimonthly IVT administrations to maintain vision and many patients fail to maintain initial visual acuity benefit due to undertreatment. The need for repeated injections can become a substantial burden for patients and caregivers with some patients requiring regular anti-VEGF injections despite treatment for a decade. Recent studies have shown that in real-world use, many patients receive fewer than recommended injections and do not receive or maintain the same benefits shown in clinical trial settings with vision gains during the first 2 years not maintained at 5 years.

Thus, there remains a need for new or improved compounds and therapies for the treatment of angiogenic ocular diseases such as wetAMD.

### SUMMARY OF THE INVENTION

Disclosed are compositions and methods for the treatment of an ocular disease associated with ocular angiogenesis including but not limited to wet (neovascular, exudative) age-related macular degeneration; macular edema following retinal vein occlusion; retinal neovascularization resulting from retinal vein occlusion; diabetic macular edema, diabetic retinopathy (including all stages of non-proliferative diabetic retinopathy and proliferative diabetic retinopathy); myopic macular degeneration; branch retinal vein occlusion, hemi-retinal vein occlusion, and central retinal vein occlusion; retinopathy of prematurity; idiopathic choroidal neovascularization; myopia macular degeneration and secondary retinal and choroidal neovascularization; retinal telangiectasia; neovascular glaucoma; vitreous hemorrhage; retinal and choroidal neovascularization secondary to retinal diseases, including but not limited to uveitis, trauma, retinal degenerative disorders, genetic retinal and/or choroidal disease, tumors of the eye, corneal and iris neovascularization. In some embodiments, the angiogenic ocular disease is selected from wet (neovascular, exudative) age-related macular degeneration; diabetic macular edema; macular edema following retinal vein occlusion; diabetic retinopathy; and myopic choroidal neovascularization.

In some embodiments, a nucleic acid is provided comprising (i) a nucleotide sequence encoding a first anti-angiogenic polypeptide (e.g., aflibercept) and (ii) a nucleotide sequence encoding one or more interfering RNA molecule(s) that reduce expression of one or more pro-angiogenic target genes. In some aspects, the RNA molecule is a short hairpin RNA (shRNA). In other aspects, the RNA molecule is a primary miRNA molecule. In some aspects, the nucleic acid comprises an expression cassette comprising (i) a nucleotide sequence encoding a first anti-angiogenic polypeptide, operably linked to an expression control sequence and (ii) a nucleotide sequence encoding an interfering RNA molecule that reduces expression of one or more pro-angiogenic target genes, operably linked to an expression control sequence. In some embodiments, the nucleotide sequence encoding the anti-angiogenic polypeptide and the nucleotide sequence encoding the interfering RNA molecule are operably linked to distinct expression control sequences. In preferred embodiments, expression of the anti-angiogenic polypeptide and the interfering RNA molecule are driven by a common (i.e., the same) expression control sequence. In some aspects, the expression control sequence(s) comprise(s) a constitutive promoter such as a CAG or CBA promoter. In other aspects, the expression control sequence(s) comprise(s) a cell-specific promoter.

In some embodiments, the nucleic acid comprises nucleotide sequence encoding an interfering RNA molecule that targets angiopoietin-2 (aka Ang2 or Ang-2). Representative human Ang2 sequences can be found at e.g., NCBI Accession No. 015123 and SEQ ID Nos: 517 and 518 of U.S. Pat. No. 8,987,420, the contents of which are incorporated herein by reference. In preferred embodiments, the interfering RNA molecule targets Ang-2 and comprises a sense strand and an antisense strand comprising, consisting essentially of, or consisting of a sequence selected from those listed in Table 1 below:

TABLE 1
Interfering RNAs targeting human Ang-2

| | | Reference |
|---|---|---|
| | | Sequence |
| | | Target |
| | | Region |
| | | (NCBI |
| | | Accession |
| Sense | Antisense | Number; |
| Strand | Strand | NM_ |
| Sequence | Sequence | 001118887.2) |
| GCCGCAGCCTATAAC | AAAGTTGTTATAGGC | GCCGCAGCCTATAAC |
| AACTTT | TGCGGC | AACTTT |
| (SEQ ID NO: 1) | (SEQ ID NO: 2) | (SEQ ID NO: 3) |
| CCCTAATTCTACAGA | ATCTCTTCTGTAGAA | CCCTAATTCTACAGA |
| AGAGAT | TTAGGG | AGAGAT |
| (SEQ ID NO: 4) | (SEQ ID NO: 5) | (SEQ ID NO: 6) |
| GATGATAGAAATAGG | TTTGTCCCTATTTCT | GATGATAGAAATAGG |
| GACAAA | ATCATC | GACAAA |
| (SEQ ID NO: 7) | (SEQ ID NO: 8) | (SEQ ID NO: 9) |
| GCCACGGTGAATAAT | AACTGAATTATTCAC | GCCACGGTGAATAAT |
| TCAGTT | CGTGGC | TCAGTT |
| (SEQ ID NO: 10) | (SEQ ID NO: 11) | (SEQ ID NO: 12) |
| GCTTACTCATTGTAT | ATGTTCATACAATGA | GCTTACTCATTGTATG |
| GAACAT | GTAAGC | AACAT |
| (SEQ ID NO: 13) | (SEQ ID NO: 14) | (SEQ ID NO: 15) |

In some embodiments, the interfering RNA molecule comprises a sense strand and an antisense strand, one or both of which comprises, consists essentially of, or consists of a sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 910%, at least 92%, at least 93%, at least 94%, at least 95%, at least 96%, at least 97%, at least 98%, or at least 99% identical to a sequence selected from those listed in Table 1. In some particularly preferred embodiments, the nucleic acid comprises nucleotide sequence encoding a first anti-angiogenic polypeptide that is aflibercept and nucleotide sequence encoding an interfering RNA molecule targets human angiopoietin-2.

In some preferred embodiments, the nucleic acid comprises nucleotide sequence encoding an interfering RNA molecule that targets VEGF-C. Representative human VEGF-C sequences can be found at e.g., GenBank Accession numbers NM . . . 005429 and X94216. In preferred embodiments, the interfering RNA molecule targets VEGF-C and comprises a sense strand and an antisense strand comprising, consisting essentially of, or consisting of a sequence selected from those listed in Table 2 below:

TABLE 2
Interfering RNAs targeting human VEGF-C

| | | Reference |
|---|---|---|
| | | Sequence |
| | | Target |
| | | Region |
| | | (NCBI |
| | | Accession |
| Sense | Antisense | Number: |
| Strand | Strand | NM_ |
| Sequence | Sequence | 005429.5) |
| CGCGACAAACACCTT | TTAAAGAAGGTGTTT | CGCGACAAACACCTT |
| CTTTAA | GTCGCG | CTTTAA |
| (SEQ ID NO: 16) | (SEQ ID NO: 17) | (SEQ ID NO: 18) |
| CTACCTCAGCAAGAC | AATAACGTCTTGCTG | CTACCTCAGCAAGAC |
| GTTATT | AGGTAG | GTTATT |
| (SEQ ID NO: 19) | (SEQ ID NO: 20) | (SEQ ID NO: 21) |
| ACCAATTACATGTGG | ATTATTCCACATGTA | ACCAATTACATGTGG |
| AATAAT | ATTGG | AATAAT |
| (SEQ ID NO: 22) | (SEQ ID NO: 23) | (SEQ ID NO: 24) |
| ATGACCAAACAGCCA | AAATCTTGGCTGTTT | AATGACCAAACAGCC |
| AGATTT | GGTCA | AAGATTT |
| (SEQ ID NO: 25) | (SEQ ID NO: 26) | (SEQ ID NO: 27) |
| GTCGTTGTGTCCCTT | AATATGAAGGGACAC | GTCGTTGTGTCCCTT |
| CATATT | AACGAC | CATATT |
| SEQ ID NO: 28) | (SEQ ID NO: 29) | (SEQ ID NO: 30) |

In some embodiments, the interfering RNA molecule comprises a sense strand and an antisense strand, one or both of which comprises, consists essentially of, or consists of a sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to a sequence selected from those listed in Table 2. Additional interfering RNA molecules targeting VEGF-C include those listed at Table 1 of US Patent Application Publication No. 2011/0293625A1, the contents of which are incorporated herein by reference. In some particularly preferred embodiments, the nucleic acid comprises nucleotide sequence encoding a first anti-angiogenic polypeptide that is aflibercept and nucleotide sequence encoding an interfering RNA molecule targets human VEGF-C.

In related embodiments, the nucleic acid comprises nucleotide sequence encoding a first anti-angiogenic protein (e.g., aflibercept) and further comprises an interfering RNA molecule that targets human VEGF-C and an interfering RNA molecule that targets human Ang-2.

In other embodiments, the nucleic acid comprises a nucleotide sequence encoding an interfering RNA molecule that targets VEGFR-3. Representative human VEGFR-3 sequences can be found e.g., at GenBank Accession Number X68203. In preferred embodiments, the interfering RNA molecule targets VEGFR-3 and comprises a sense strand and an antisense strand comprising, consisting essentially of, or consisting of a sequence selected from those listed in Table 3 below:

TABLE 3
Interfering RNAs targeting human VEGFR-3

| | | Reference Sequence Target |
|---|---|---|
| | | Region |
| | | (NCBI Accession Number: |
| Sense Strand Sequence | Antisense Strand Sequence | NM_001354989.2) |
| ACAACGGCATCCAGCGA | GAAATCGCTGGATGCCG | ACAACGGCATCCAGCGA |
| TTTC | TTG | TTTC (SEQ ID NO: 33) |
| (SEQ ID NO: 31) | (SEQ ID NO: 32) | |
| GGACACCCTGCAAGATG | CAAACATCTTGCAGGGT | GGACACCCTGCAAGATG |
| TTTG | GTCC | TTTG |
| (SEQ ID NO: 34) | (SEQ ID NO: 35) | (SEQ ID NO: 36) |
| CACCGTGTGGGCTGAGT | TTAAACTCAGCCCACAC | CACCGTGTGGGCTGAGT |
| TTAA | GGTG | TTAA |
| (SEQ ID NO: 37) | SEQ ID NO: 38) | (SEQ ID NO: 39) |
| CTTTAAGACTTTCGCTAT | GAAATAGCGAAAGTCTT | CTTTAAGACTTTCGCTAT |
| TTC | AAAG (SEQ ID NO: 41) | TTCG |
| SEQ ID NO: 40) | | (SEQ ID NO: 42) |
| TCACAGGCAACGAGCTC | CATAGAGCTCGTTGCCTG | TCACAGGCAACGAGCTC |
| TATG | TGA | TATG |
| (SEQ ID NO: 43) | (SEQ ID NO: 44) | (SEQ ID NO: 45) |

In some embodiments, the interfering RNA molecule comprises a sense strand and an antisense strand, one or both of which comprises, consists essentially of, or consists of a sequence at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to a sequence selected from those listed in Table 3. Additional interfering RNA molecules targeting VEGFR-3 include those listed at Table 2 of U.S. Pat. No. 7,517,864, the contents of which are incorporated herein by reference. In some particularly preferred embodiments, the nucleic acid comprises nucleotide sequence encoding a first anti-angiogenic polypeptide that is aflibercept and nucleotide sequence encoding an interfering RNA molecule targets human VEGFR-3.

In some embodiments, the synthetic RNA molecule is a small interfering RNA (siRNA). In some embodiments, the interfering RNA is a small hairpin RNA (shRNA). In some aspects, the shRNA has a loop comprising (5′ to 3′) the sequence CTCGAG or a sequence at least 70% or at least 80% identical thereto.

In some preferred embodiments, the synthetic RNA molecule is an artificial micro RNA (miRNA). In some embodiments, the artificial miRNA comprises a sense strand and antisense strand as herein described embedded into an miRNA “scaffold” derived from miR-30, miR-22, miR-15, miR-16, miR-103 or miR-107. In some preferred aspects, the artificial miRNA comprises a sense and antisense strand as herein described embedded into mir-30:

(SEQ ID NO: 46)
CUUCAGGUUAACCCAACAGAAGGCUAAAGAAGGUAUAUUGCUGUUGAC
AGUGAGCG(X)ₙCUGUGAAGCCACAGAUGGG(Y)ₙ
UGCCUACUGCCUCGGACUUCAAGGGGCUACUUUAGG,

wherein (X)ₙ comprises a sense strand and (Y)ₙ comprises a sense strand from any one of Tables 1-3.

In particularly preferred aspects, the artificial miRNA comprises a sense and antisense strand as herein described embedded into mir-E:

(SEQ ID NO: 47)
GACUUCUUAACCCAACAGAAGGCUCGAGAAGGUAUAUUGCUGUUGACA
GUGAGCG(X)ₙUAGUGAAGCCACAGAUGUA(Y)ₙ
UGCCUACUGCCUCGGACUUCAAGGGGCUAGAAUUC,

wherein (X)ₙ comprises a sense strand and (Y)ₙ comprises a sense strand from any one of Tables 1-3.

In some embodiments, a nucleic acid is provided comprising (i) nucleotide sequence encoding a first anti-angiogenic polypeptide (e.g., aflibercept) and (ii) nucleotide sequence encoding a second anti-angiogenic polypeptide. In some aspects, the nucleic acid comprises an expression cassette comprising (i) nucleotide sequence encoding a first anti-angiogenic polypeptide, operably linked to an expression control sequence and (ii) nucleotide sequence encoding a second anti-angiogenic polypeptide, operably linked to an expression control sequence. In some embodiments, the nucleotide sequence encoding the first anti-angiogenic polypeptide and the nucleotide sequence encoding the second anti-angiogenic polypeptide are operably linked to distinct expression control sequences. In preferred embodiments, expression of the first and second anti-angiogenic polypeptides are driven by a common (i.e., the same) expression control sequence. In some aspects, the expression control sequence(s) comprise(s) a constitutive promoter such as a CAG or CBA promoter. In other aspects, the expression control sequence(s) comprise(s) a cell-specific promoter.

In some aspects, the first and/or second anti-angiogenic polypeptide (i.e., a polypeptide that inhibits angiogenesis) is selected from endostatin; tumstatin; angiostatin; pigment epithelium-derived factor (PEDF). In some aspects, the first and/or second anti-angiogenic polypeptide is a “decoy” fusion protein (e.g., a soluble receptor fusion protein) that binds to and inhibits the activity of a VEGF (VEGF-A (see e.g., GenBank Acc. No. Q16889), VEGF-B (see e.g., GenBank Acc. No. U48801), VEGF-C (see e.g., GenBank Acc. No. X94216), VEGF-D (see e.g., GenBank Acc. No. A000185) and/or placenta growth factor (PIGF; see e.g., GenBank Acc. No. X54936)), representative examples of which include soluble VEGFR-1 (aka Flt-1; see e.g., GenBank Acc. No. X51602) receptor fusion proteins, soluble VEGFR-2 (aka Flk-1; see e.g., GenBank Acc. No. X59397) receptor fusion proteins, soluble VEGFR-3 (aka Flt-4; see e.g., GenBank Acc. Nos. X68203 and S66407) receptor fusion proteins and chimeric soluble receptor fusion proteins comprising binding regions from at least two of VEGFR-1, VEGFR-2 and VEGFR-3. VEGF-A binds to VEGFR-1 and VEGFR-2; VEGF-B and PIGF bind to VEGFR-2; VEGF-C and VEGF-D bind to VEGFR-3.

In some preferred aspects, the first anti-angiogenic polypeptide is a soluble fusion protein comprising VEGF-binding portions from the extracellular domains of VEGFR-1 and VEGFR-2, optionally fused to a human IgG1 Fc portion. In particularly preferred aspects, the first anti-angiogenic polypeptide is aflibercept. Aflibercept is a recombinant fusion protein consisting of VEGF-binding portions from the extracellular domains of human VEGFR-1 and VEGFR-2 fused to a human IgG1 Fc portion. Aflibercept is indicated for the treatment of neovascular (wet) age-related macular degeneration, macular edema following retinal vein occlusion, diabetic macular edema and diabetic retinopathy.

In other preferred aspects, the first and/or second anti-angiogenic polypeptide is a soluble fusion protein comprising one or more VEGF-binding portions from the extracellular domain of VEGFR-3.

In other aspects, the first and/or second anti-angiogenic polypeptide is an antibody or antigen-binding fragment thereof that binds to and inhibits the activity of a pro-angiogenic protein such as a VEGF and/or an angiopoietin (angiopoietin-1/Ang1/Ang-1, angiopoietin-2/Ang2/Ang-2). In some aspects, the first and/or second anti-angiogenic polypeptide is an antibody against Ang1 and/or Ang2. In other aspects the first and/or second anti-angiogenic polypeptide is an antibody against VEGF-A (e.g., bevacizumab), VEGF-B, VEGF-C, VEGF-D, or PIGF (e.g., TB-403, 16D3) that blocks binding of the VEGF to its cognate receptor. In other aspects, the first and/or second anti-angiogenic polypeptide is an antibody against VEGFR-1 (e.g., icrucumab, D16F7, KM1730/KM1732), VEGFR-2 (e.g., ramucirumab), or VEGFR-3 that blocks binding of the receptor to a VEGF. In some aspects, the antibody is a bifunctional antibody. In some preferred embodiments, the first and/or second anti-angiogenic polypeptide is an antibody or antigen binding fragment thereof that binds to Ang-2.

In some embodiments, the nucleic acid comprises nucleotide sequence encoding a first anti-angiogenic polypeptide and nucleotide sequence encoding a second anti-angiogenic polypeptide, wherein the second anti-angiogenic polypeptide is pigment epithelium-derived factor (PEDF).

In some embodiments, provided herein is a vector (e.g., an adeno-associated virus (AAV) plasmid vector) comprising a nucleic acid as herein described (e.g., comprising nucleotide sequence encoding a first and/or second anti-angiogenic polypeptide and/or an interfering RNA interfering RNA molecule that reduces expression of one or more pro-angiogenic target genes). In preferred embodiments, the vector is a recombinant adeno-associated (rAAV) expression vector. In some embodiments, the rAAV vector comprises a native capsid (e.g., a capsid of AAV serotype 2 or AAV serotype 5 or AAV serotype 8). In other embodiments, the rAAV vector comprises a capsid that is modified (e.g., comprises one or more peptide insertions and/or one or more amino acid substitutions (e.g., tyrosine to phenylalanine) and/or amino acid insertions or amino acid deletions) relative to a native AAV capsid (e.g., comprising one or more modifications relative to an AAV capsid of serotype 2, 5 or 8).

In preferred embodiments, the rAAV vector comprises a capsid with a variant capsid protein comprising the following amino acid sequence or a sequence at least 80%, 90%, 95% or 99% identical thereto:

(SEQ ID NO: 48)
MAADGYLPDWLEDTLSEGIRQWWKLKPGPPPPKAAERHKDDSRGLVLPG
YKYLGPFNGLDKGEPVNEADAAALEHDKAYDRQLDSGDNPYLKYNHADA
EFQERLKEDTSFGGNLGRAVFQAKKRVLEPLGLVEEPVKTAPGKKRPVE
HSPVEPDSSSGTGKAGQQPARKRLNFGQTGDADSVPDPQPLGQPPAAPS
GLGTNTMATGSGAPMADNNEGADGVGNSSGNWHCDSTWMGDRVITTSTR
TWALPTYNNHLYKQISSQSGASNDNHYFGYSTPWGYFDFNRFHCHFSPR
DWQRLINNNWGFRPKRLNFKLFNIQVKEVTQNDGTTTIANNLTSTVQVF
TDSEYQLPYVLGSAHQGCLPPFPADVFMVPQYGYLTLNNGSQAVGRSSF
YCLEYFPSQMLRTGNNFTFSYTFEDVPFHSSYAHSQSLDRLMNPLIDQY
LYYLSRTNTPSGTTTQSRLQFSQAGASDIRDQSRNWLPGPCYRQQRVSK
TSADNNNSEYSWTGATKYHLNGRDSLVNPGPAMASHKDDEEKFFPQSGV
LIFGKQGSEKTNVDIEKVMITDEEEIRTTNPVATEQYGSVSTNLQRGNL
AISDQTKHARQAATADVNTQGVLPGMVWQDRDVYLQGPIWAKIPHTDGH
FHPSPLMGGFGLKHPPPQILIKNTPVPANPSTTFSAAKFASFITQYSTG
QVSVEIEWELQKENSKRWNPEIQYTSNYNKSVNVDFTVDTNGVYSEPRP
IGTRYLTRNL

The variant AAV capsid protein of SEQ ID NO:48 contains the following modifications relative to native AAV2 capsid: (i) a proline (P) to alanine (A) mutation at amino acid position 34, which is located inside the assembled capsid (VP1 protein only), and (ii) an insertion of 10 amino acids (leucine-alanine-isoleucine-serine-aspartic acid-glutamine-threonine-lysine-histidine-alanine/LAISDQTKHA (SEQ ID NO:49)) at amino acid position 588, which is present in VP1, VP2, and VP3. In some embodiments, the capsid comprises a variant capsid protein comprising a sequence at least 90%, at least 95%, at least 98%, at least 99% identical to SEQ ID NO:48 and comprising a P34A substitution and an LAISDQTKHA (SEQ ID NO:49) peptide insertion in the GH loop of the capsid, e.g., between two adjacent amino acids at a position between amino acids 570 and 611 of VP1, preferably between amino acids 588 and 589 of VP1 (numbering is relative to native AAV2 VP1 capsid).

In another embodiment, provided herein is a host cell comprising a nucleic acid as herein described. In some aspects, the host cell is a mammalian cell, including without limitation, a CHO cell, an HEK293 cell, a HeLa cell, a BHK21 cell, a Vero cell or a V27 cell. In other aspects, the host cell is a photoreceptor cell (e.g., rods; cones), a retinal ganglion cell (RGC), a glial cell (e.g., a Müller glial cell, a microglial cell), a bipolar cell, an amacrine cell, a horizontal cell, or a retinal pigmented epithelium (RPE) cell.

In some embodiments, the disclosure provides a method of treating an ocular disease associated with ocular angiogenesis in a subject (e.g. a human subject) comprising administering to the subject a nucleic acid molecule or vector as described herein.

### DESCRIPTION OF THE DRAWINGS

FIGS. 1A-B Dual transgene construction. Approaches to building multi-mechanistic anti-angiogenic gene therapy are shown. FIG. 1A: this construct is a representative embodiment of a nucleic acid encoding aflibercept and a second anti-angiogenic protein. FIG. 1B: this construct is a representative embodiment of a nucleic acid encoding aflibercept and an interfering RNA targeting a pro-angiogenic gene. A ubiquitous promoter (CBA) is employed in both constructs.

FIG. 2 Depiction of Representative Constructs. pP141.001 comprises a CAG promoter with a miR-E-Ang2 sequence placed within the CAG intron (well after the splice donor site) followed by codon-optimized sequence encoding aflibercept. pP145.001 is identical to pP141.001 except that a mir-E-VEGF-C sequence is placed within the CAG intron. pP151.001 is identical to pP145.001 except that a miR-E-Ang2 sequence is placed within the 3′ UTR of the aflibercept gene. pP151.002 is identical to pP145.001 except that a miR-E-Ang2 sequence is placed within the aflibercept coding sequence. pP153.001 comprises a CAG promoter with miR-E-Ang2 and miR-E-VEGF-C sequences placed within the CAG intron (followed by codon-optimized sequence encoding aflibercept). CBA Prom.: ubiquitous chicken b-actin promoter, CBA Ex1/Int1: chicken b-actin exon 1/hybrid chicken b-actin and rabbit beta globulin intron, RGB SA/PPT: rabbit beta globulin exon 3 fragment (splice acceptor)/polypyrimidine tract, T2A: self cleaving peptide, AFLB: aflibercept.

FIGS. 3A-B FIG. 3A is a Western blot using anti-Human IgG Fe to detect aflibercept in HEK293T media following transfection with AAV plasmid comprising nucleotide sequence encoding aflibercept along with a second polypeptide (PEDF, VEGFR3, anti-Ang2 scFab (LH and HL)) with both sequences under control of a CBA promoter. FIG. 3B is a graph illustrating expression of aflibercept in HEK293T media following transfection with AAV plasmid encoding aflibercept and a second polypeptide (PEDF, anti-Ang2 scFab) or encoding I1 aflibercept and an interfering RNA under control of the indicated promoter. Results are normalized to expression of aflibercept in HEK293T media following transfection with AAV plasmid encoding aflibercept alone under the control of a CBA promoter. Error represents S.E.M. N=6 Biological replicates. Analyzed by one-way ANOVA. ****: P<0.0001. Constructs only weighed statistically against their appropriate control. % Shows mean difference compared to CBA-AFLB.

FIG. 4 illustrates free and active aflibercept (AFLB) in the media eight days after transduction of RPE cells with rAAV comprising a capsid of SEQ ID NO:48 and heterologous nucleic acid encoding the indicated transgenes at the specified MOI (N=3 biological replicates per conditions; statistics only calculated for matched MOIs).

FIGS. 5A-C FIGS. 5A and 5B illustrate free and active aflibercept (AFLB) in the media seven (FIG. 5A) and eleven (FIG. 5B) days after transduction of RPE cells with rAAV comprising a capsid of SEQ ID NO:48 and heterologous nucleic acid encoding the indicated transgenes at MOI of 5K and 1K. FIG. 5C compares AFLB following transduction of RPE cells with a rAAV comprising a construct encoding AFLB only with rAAV comprising a construct encoding AFLB+RNAi against VEGF-C.

FIGS. 6A-B illustrate time course comparisons of free and active AFLB in media of RPE cells following transduction with a rAAV comprising a construct encoding AFLB only with rAAV comprising a construct encoding AFLB+RNAi against VEGF-C.

FIGS. 7A-B illustrate a comparison of VEGF-A neutralization (assessed by ELISA) in RPE supernatant at day 7 (FIG. 7A) and day 11 (FIG. 7B) following transduction at the specified MOIs with rAAV comprising a capsid protein of SEQ ID NO:48 and the indicated constructs.

FIGS. 8A-D illustrate expression of functional anti-Ang2 scFab from dual protein constructs (plasmid encoding AFLB+anti-Ang2 scFab in LH or HL configuration) following transfection of HEK293T cells (assessed by competition ELISA and Tie2 receptor competition assays.

FIG. 9 illustrates anti-Ang2 scFab (and anti-AFLB) Western blot results from media of RPE cells following transduction with rAAV comprising capsid protein of SEQ ID NO:48 and heterologous nucleic acid encoding AFLB+anti-Ang2 scFab (HL and LH conformations) at MOI 5000 and 1000, with similar expression levels of LH and HL forms observed.

FIGS. 10A-B illustrate the results of anti-Ang2 functional ELISA (ANG2-coated plates) (FIG. 10A) and competition ELISA (FIG. 10B) on media from RPE cells eleven days after transduction with rAAV as described in FIG. 9 at MOI of 5000 and 1000.

FIG. 11 illustrates binding affinities of anti-Ang2 scFab (assessed by Biacore) encoded by the rAAV as described in FIG. 9.

FIGS. 12A-B illustrate PEDF expression from dual protein constructs (encoding AFLB+PEDF) following plasmid transfection of HEK293T cells

FIGS. 13A-B illustrate VEGFR3-Fc expression from dual protein constructs (encoding AFLB+VEGFR3-Fc) following plasmid transfection of HEK293T cells. Dramatic loss of aflibercept is shown by ELISA, resulting from heterodimerization.

FIG. 14 illustrates constructs pP143.001 (miRNA-only efficacy control) and pP141.001, encoding Ang2 miR-E (RNAi sense and antisense strands embedded within miR-E backbone) and RPF657 (pP143.001) or AFLB (pP141.001).

FIGS. 15A-B illustrate kD of Ang2 by ELISA (secreted and cellular) following transduction of HUVECs with the indicated shAng2 constructs, normalized to control (FIG. 15A) or non-normalized (FIG. 15B)

FIGS. 16A-B FIG. 16A illustrates results of Ang2 ELISA (secreted and cellular) following transduction of human RMVEC cells with rAAV comprising a capsid protein of SEQ ID NO:48 and a nucleic acid encoding miRNA comprising the sense and antisense strands of shRNA #5 (as described in FIGS. 15A-B) embedded within an miR-E backbone. FIG. 16B illustrates a significant decrease in Ang2 secretion from RMVEC assessed by Ang2 qPCR.

FIGS. 17A-B illustrate Ang2 RNA levels (RT-qPCR; FIG. 17A) and protein levels (ELISA; FIG. 17B) in human RPE cells eight days after transduction with rAAV as described in FIG. 16 at the specified MOIs (rAAV encoding AFLB only or GFP served as controls).

FIGS. 18A-B illustrate free AFLB protein levels (FIG. 18A) and AFLB mRNA levels (FIG. 18B) in RMVEC cells eight days after transduction at the specified MOIs with rAAV comprising a capsid protein of SEQ ID NO:48 and the specified nucleic acid constructs.

FIGS. 19A-C illustrate Ang2 secretion (FIG. 19A) and Ang2 RNA levels (FIGS. 19B and 19C) in RMVEC cells eight days after transduction with rAAV comprising a capsid protein of SEQ ID NO:48 and the specified nucleic acid constructs.

FIG. 20 illustrates the percentage of VEGF-C RNA levels (normalized to RPL32; RNA analysis by qPCR) in HEK293T cells following transduction with the indicated shVEGF-C constructs.

FIGS. 21A-C illustrate VEGF-C protein (by ELISA; FIG. 21A) and VEGF-C mRNA (by qPCR; FIG. 21B) following transduction of human RPE cells at the specified MOI with rAAV comprising a capsid protein of SEQ ID NO:48 and the specified nucleic acid constructs. FIG. 21C illustrates a dose-dependent increase in expression of miRNA targeting VEGF-C in the cells.

FIGS. 22A-C illustrate endogenous VEGF-A neutralization (FIG. 22A) and VEGF-C protein (FIG. 22B) and mRNA levels (FIG. 22C) in RPE cells eight days after transduction at the specified MOIs with rAAV comprising a capsid protein of SEQ ID NO:48 and the specified nucleic acid constructs

FIG. 23 illustrates histopathological staining for GFP and CD31 colocalization in retinal endothelial cells from NHP eyes following intravitreal injection with 1.2×10¹² vg of rAAV comprising a capsid protein of SEQ ID NO:48 (R100) and nucleic acid encoding GFP.

FIG. 24 illustrates aflibercept (AFLB) expression in the aqueous, vitreous and retina+choroid of NHPs following intravitreal administration of rAAV comprising a capsid protein of SEQ ID NO:48 and a nucleic acid encoding aflibercept and an miRNA targeting VEGF-C.

FIG. 25 illustrates miRNA copies in the retina of NHPs (as described in FIG. 24, left panel) and MiSeq data confirming miRNA targeting VEGF-C as the predominant miRNA species in the NHP retinas.

FIG. 26 illustrates the protective effect of rAAV comprising a capsid protein of SEQ ID NO:48 and a nucleic acid encoding aflibercept and an miRNA targeting VEGF-C, administered at the specified doses, compared to vehicle control in an NHP model of choroidal neovascularization.

FIG. 27 illustrates cell proliferation (left) and migration (right) of HUVEC cells following electroporation with plasmids encoding (i) aflibercept and an miRNA targeting VEGF-C (ii) aflibercept only or (iii) GFP only.

FIG. 28 illustrates aflibercept concentration (ng/ml) in aqueous humor of NHPs following a single intravitreal administration at the specified dose of (i) rAAV comprising a capsid protein of SEQ ID NO:48 and a nucleic acid encoding aflibercept and miR targeting VEGF-C (comprising the sense and antisense strands corresponding to SEQ ID Nos: 19 and 20 and the full construct corresponding to SEQ ID NO:69) or (ii) rAAV comprising a capsid protein of SEQ ID NO:48 and a nucleic acid encoding aflibercept only.

FIG. 29 illustrates intraocular inflammation in NHPs following a single intravitreal administration at the specified dose of (i) rAAV comprising a capsid protein of SEQ ID NO:48 and a nucleic acid encoding aflibercept and miR targeting VEGF-C (comprising the sense and antisense strands corresponding to SEQ ID Nos: 19 and 20 and the full construct corresponding to SEQ ID NO:69) or (ii) rAAV comprising a capsid protein of SEQ ID NO:48 and a nucleic acid encoding aflibercept only, at the specified timepoints as assessed by slit lamp biomicroscopy compared to vehicle-treated control eyes.

FIG. 30 illustrates mean values of the sum of retinal volume and average central retinal thickness in NHPs from baseline to 22 weeks following a single intravitreal administration at the specified dose of (i) rAAV comprising a capsid protein of SEQ ID NO:48 and a nucleic acid encoding aflibercept and miR targeting VEGF-C (comprising the sense and antisense strands corresponding to SEQ ID Nos: 19 and 20 and the full construct corresponding to SEQ ID NO:69) or (ii) rAAV comprising a capsid protein of SEQ ID NO:48 and a nucleic acid encoding aflibercept only.

FIG. 31 illustrates the results of full field electroretinography (ffERG) at day 84 and week 22 in NHPs following a single intravitreal administration at the specified dose of (i) rAAV comprising a capsid protein of SEQ ID NO:48 and a nucleic acid encoding aflibercept and miR targeting VEGF-C (comprising the sense and antisense strands corresponding to SEQ ID Nos: 19 and 20 and the full construct corresponding to SEQ ID NO:69) or (ii) rAAV comprising a capsid protein of SEQ ID NO:48 and a nucleic acid encoding aflibercept only.

### DETAILED DESCRIPTION OF THE INVENTION

### Definitions

A “codon adaptation index,” as used herein, refers to a measure of codon usage bias. A codon adaptation index (CAI) measures the deviation of a given protein coding gene sequence with respect to a reference set of genes (Sharp P M and Li W H, Nucleic Acids Res. 15(3):1281-95 (1987)). CAI is calculated by determining the geometric mean of the weight associated to each codon over the length of the gene sequence (measured in codons):

$$CAI = exp ( 1 / L {\sum }_{i = 1}^{L} ln ( {w}_{1} ( l ) ) ) , \qquad ( I )$$

For each amino acid, the weight of each of its codons, in CAI, is computed as the ratio between the observed frequency of the codon (fi) and the frequency of the synonymous codon (fj) for that amino acid:

$${w}_{1} = \frac{{f}_{i}}{max ( {f}_{j} )} ij ∈ [ synonymous codons for amino acid ] \qquad ( II )$$

The term “isolated” designates a biological material (cell, nucleic acid or protein) that has been removed from its original environment (the environment in which it is naturally present). For example, a polynucleotide present in the natural state in a plant or an animal is not isolated, however the same polynucleotide separated from the adjacent nucleic acids in which it is naturally present, is considered “isolated.”

As used herein, a “coding region” or “coding sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide. Two or more coding regions can be present in a single polynucleotide construct, e.g., on a single vector, or in separate polynucleotide constructs, e.g., on separate (different) vectors. It follows, then that a single vector can contain just a single coding region or can comprise two or more coding regions.

A “2A peptide” refers to “self-cleaving” peptides of about 20 amino acids that produce equimolar levels of multiple genes from the same mRNA and may be used in place of IRES elements in multicistronic vectors. Non-limiting examples include T2A, P2A, E2A and F2A peptides sequences. In embodiments wherein a heterologous nucleic acid comprises nucleotide sequence encoding multiple gene products, expression of the multiple (e.g. 2) gene products can be mediated by multiple (e.g. 2) independent promoters or may be mediated by a single promoter, with the multiple transgenes separated by an internal ribosome entry site (IRES) or a 2A peptide sequence.

As used herein, the term “regulatory region” refers to nucleotide sequences located upstream (5′ non-coding sequences), within, or downstream (3′ non-coding sequences) of a coding region, and which influence the transcription, RNA processing, stability, or translation of the associated coding region. Regulatory regions can include promoters, translation leader sequences, introns, polyadenylation recognition sequences, RNA processing sites, effector binding sites and stem-loop structures. If a coding region is intended for expression in a eukaryotic cell, a polyadenylation signal and transcription termination sequence will usually be located 3′ to the coding sequence.

As used herein, the term “nucleic acid” is interchangeable with “polynucleotide” or “nucleic acid molecule” and a polymer of nucleotides is intended.

A polynucleotide which encodes a gene product, e.g., a polypeptide, can include a promoter and/or other transcription or translation control elements operably associated with one or more coding regions. In an operable association a coding region for a gene product, e.g., a polypeptide, is associated with one or more regulatory regions in such a way as to place expression of the gene product under the influence or control of the regulatory region(s). For example, a coding region and a promoter are “operably associated” if induction of promoter function results in the transcription of mRNA encoding the gene product encoded by the coding region, and if the nature of the linkage between the promoter and the coding region does not interfere with the ability of the promoter to direct the expression of the gene product or interfere with the ability of the DNA template to be transcribed. Other transcription control elements, besides a promoter, for example enhancers, operators, repressors, and transcription termination signals, can also be operably associated with a coding region to direct gene product expression.

“Transcriptional control sequences” or “expression control sequences” refer to DNA regulatory sequences, such as promoters, enhancers, terminators, and the like, that provide for the expression of a coding sequence in a host cell. A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit beta-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

A “CAG promoter” is composed of (C) the cytomegalovirus (CMV) early enhancer element, (A) the promoter, the first exon and the first intron of chicken beta-actin gene, (G) the splice acceptor of the rabbit beta-globin gene. See Miyazaki, J., Takaki, S., Araki, K., Tashiro, F., Tominaga, A., Takatsu, K., & Yamamura, K. (1989). Expression vector system based on the chicken β-actin promoter directs efficient production of interleukin-5. Gene, 79(2), 269-277, the contents of which are incorporated herein by reference.

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

The term “expression” as used herein refers to a process by which a polynucleotide produces a gene product, for example, an RNA or a polypeptide. It includes without limitation transcription of the polynucleotide into messenger RNA (mRNA), transfer RNA (tRNA), primary miRNA, small hairpin RNA (shRNA), small interfering RNA (siRNA), or any other RNA product, and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage.

A “vector” refers to any vehicle for the cloning of and/or transfer of a nucleic acid into a host cell. A vector can be a replicon to which another nucleic acid segment can be attached so as to bring about the replication of the attached segment. The term “vector” includes both viral and nonviral vehicles for introducing the nucleic acid into a cell in vitro, ex vivo or in vivo. A large number of vectors are known and used in the art including, for example, plasmids, modified eukaryotic viruses, or modified bacterial viruses. Insertion, of a polynucleotide into a suitable vector can be accomplished by ligating the appropriate polynucleotide fragments into a chosen vector that has complementary cohesive termini.

Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters known and used in the art include: luciferase (Luc), green fluorescent protein (GFP), chloramphenicol acetyltransferase (CAT), -galactosidase (LacZ), -glucuronidase (Gus), and the like. Selectable markers can also be considered to be reporters.

Eukaryotic viral vectors that can be used include, but are not limited to, adenovirus vectors, retrovirus vectors, adeno-associated virus vectors, poxvirus, e.g., vaccinia virus vectors, baculovirus vectors, or herpesvirus vectors. Non-viral vectors include plasmids, liposomes, electrically charged lipids (cytofectns), DNA-protein complexes, and biopolymers.

“Promoter” and “promoter sequence” are used interchangeably and refer to a DNA sequence capable of controlling the expression of a coding sequence or functional RNA. In general, a coding sequence is located 3′ to a promoter sequence. Promoters can be derived in their entirety from a native gene, or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths can have identical promoter activity.

The term “plasmid” refers to an extra-chromosomal element often carrying a gene that is not part of the central metabolism of the cell, and usually in the form of circular double-stranded DNA molecules. Such elements can be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

A polynucleotide or polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the world wide web at ncbi.nlm.nih.gov/BLAST/. Another alignment algorithm is FASTA, available in the Genetics Computing Group (GCG) package, from Madison, Wis., USA. Other techniques for alignment are described in Methods in Enzymology, vol. 266: Computer Methods for Macromolecular Sequence Analysis (1996), ed. Doolittle, Academic Press, Inc. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

Nucleic acids encoding a first anti-angiogenic polypeptide and an antibody or antigen-binding fragment thereof

In some embodiments, a nucleic acid is provided comprising a nucleotide sequence encoding a first and second anti-angiogenic polypeptide, wherein the first anti-angiogenic polypeptide is aflibercept and the second anti-angiogenic polypeptide is an antibody or antigen-binding fragment thereof that binds to and inhibits the activity of a pro-angiogenic protein.

A preferred nucleotide sequence encoding aflibercept, codon-optimized for expression in humans, is provided below:

(SEQ ID NO: 50)
ATGGTTTCTTACTGGGACACCGGCGTGCTGCTGTGTGCCCTGCTTTCTTGTCTGCTGCTGACCGGCTCTAGCAG
CGGCTCTGATACCGGCAGACCCTTCGTGGAAATGTACAGCGAGATCCCCGAGATCATCCACATGACCGAGGGC
AGAGAGCTGGTCATCCCTTGCAGAGTGACAAGCCCCAACATCACCGTGACTCTGAAGAAGTTCCCTCTGGACAC
ACTGATCCCCGACGGCAAGAGAATCATCTGGGACAGCCGGAAGGGCTTCATCATCAGCAACGCCACCTACAAA
GAGATCGGCCTGCTGACCTGTGAAGCCACCGTGAATGGCCACCTGTACAAGACCAACTACCTGACACACAGACA
GACCAACACCATCATCGACGTGGTGCTGAGCCCTAGCCACGGCATTGAACTGTCTGTGGGCGAGAAGCTGGTG
CTGAACTGTACCGCCAGAACCGAGCTGAACGTGGGCATCGACTTCAACTGGGAGTACCCCAGCAGCAAGCACC
AGCACAAGAAACTGGTCAACCGGGACCTGAAAACCCAGAGCGGCAGCGAGATGAAGAAATTCCTGAGCACCCT
GACCATCGACGGCGTGACCAGAAGTGACCAGGGCCTGTACACATGTGCCGCCAGCTCTGGCCTGATGACCAAG
AAAAACAGCACCTTCGTGCGGGTGCACGAGAAGGACAAGACCCACACCTGTCCTCCATGTCCTGCTCCAGAACT
GCTCGGGGGACCTTCCGTGTTCCTGTTTCCTCCAAAGCCTAAGGACACCCTGATGATCAGCAGAACCCCTGAAG
TGACCTGCGTGGTGGTGGATGTGTCCCACGAGGATCCCGAAGTGAAGTTCAATTGGTACGTGGACGGCGTGGA
AGTGCACAACGCCAAGACCAAGCCTAGAGAGGAACAGTACAATAGCACCTACAGAGTGGTGTCCGTGCTGACC
GTGCTGCACCAGGATTGGCTGAACGGCAAAGAGTACAAGTGCAAGGTGTCCAACAAGGCCCTGCCTGCTCCTA
TCGAGAAAACCATCTCCAAGGCCAAGGGCCAGCCTAGGGAACCCCAGGTTTACACACTGCCTCCAAGCAGGGA
CGAGCTGACAAAGAACCAGGTGTCCCTGACCTGCCTGGTCAAGGGCTTCTACCCTTCCGATATCGCCGTGGAAT
GGGAGAGCAATGGCCAGCCTGAGAACAACTACAAGACAACCCCTCCTGTGCTGGACAGCGACGGCTCATTCTT
CCTGTACAGCAAGCTGACAGTGGACAAGAGCAGATGGCAGCAGGGCAACGTGTTCAGCTGCTCCGTGATGCAC
GAGGCCCTGCACAACCACTACACCCAGAAGTCCCTGAGCCTGTCTCCTGGCAAA

In some embodiments, the sequence is at least 80%, at least 90%, at least 95% or at least 99% identical to the nucleotide sequence of SEQ ID NO:50 and/or comprises a stop codon (e.g. TGA) at the end of the sequence. In some embodiments, the aflibercept gene product comprises the following amino acid sequence or a sequence at least 90%, 95%, 97%, 98%, or at least 99% identical thereto:

(SEQ ID NO: 51)
MVSYWDTGVLLCALLSCLLLTGSSSGSDTGRPFVEMYSEIPEIIHMTEGRELVIPCRVTSPNITVTLKKFPL
DTLIPDGKRIIWDSRKGFIISNATYKEIGLLTCEATVNGHLYKTNYLTHRQTNTIIDVVLSPSHGIELSVGEKLVLNCTART
ELNVGIDFNWEYPSSKHQHKKLVNRDLKTQSGSEMKKFLSTLTIDGVTRSDQGLYTCAASSGLMTKKNSTFVRVHEK
DKTHTCPPCPAPELLGGPSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYTQKSLSLSPGK

In some preferred embodiments, the second anti-angiogenic polypeptide is an antibody or antigen-binding fragment that binds to and inhibits the activity of an angiopoietin (angiopoietin-1/Ang1/Ang-1, angiopoietin-2/Ang2/Ang-2). In some aspects, the second anti-angiogenic polypeptide is an antibody against Ang1 and/or Ang2. In some preferred embodiments, the second anti-angiogenic polypeptide is an antibody or antigen binding fragment thereof that binds to Ang-2. In a particularly preferred embodiment, the antibody or antigen-binding fragment thereof comprises a heavy chain variable region (HCVR) comprising heavy chain complementarity determining regions (HCDRs) of HCDR1=GYYMH (SEQ ID NO:52); HCDR2=WINPNSGGTNYAQKFQG (SEQ ID NO:53) and HCDR3=SPNPYYYDSSGYYYPGAFDI (SEQ ID NO:54) and a light chain variable region (LCVR) comprising light chain complementarity determining regions (LCDRs) of LCDR1=23. GGNNIGSKSVH (SEQ ID NO:55) LCDR2=DDSDRPS (SEQ ID NO:56) and LCDR3=QVWDSSSDHWV (SEQ ID NO:57) or comprising HCDRs and LCDRs at least 90%, at least 95%, at least 98% or at least 99% identical thereto. In a related embodiment, the antibody is a single chain Fab (scFab) fragment in LH or HL orientation. In some particularly preferred embodiments, the first anti-angiogenic polypeptide is aflibercept and the second anti-angiogenic polypeptide is an antibody or antigen-binding fragment thereof that binds to human Ang-2 (e.g., an scFab fragment).

Ang-2 promotes angiogenesis and vascular permeability. Ang-2 expression is increased, inter alia, in vitreous of diabetic macular edema (DME), wet age-related macular degeneration (wAMD) and retinal vein occlusion (RVO) patients. High Ang-2 expression is correlated with decreased BVCA (best-corrected visual acuity) and high central macular thickness (CMT) in wAMD patients.

Unless specifically indicated otherwise, the term “antibody,” as used herein, shall be understood to encompass antibody molecules comprising two immunoglobulin heavy chains and two immunoglobulin light chains (i.e., “full antibody molecules”) as well as antigen-binding fragments thereof. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding fragment” of an antibody, or “antibody fragment”, as used herein, refers to one or more fragments of an antibody that retain the ability to specifically bind to an antigen. An antibody fragment may include a Fab fragment, a F(ab′)₂ fragment, a Fv fragment, a dAb fragment, a fragment containing a CDR, or an isolated CDR. In certain embodiments, the term “antigen-binding fragment” refers to a polypeptide fragment of a multi-specific antigen-binding molecule. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and (optionally) constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments (e.g., single chain Fab (scFab) fragments); (ii) F(ab′)₂ fragments; (iii) Fd fragments; (iv) Fv fragments; (v) single-chain Fv (scFv) molecules; (vi) dAb fragments (single domain antibody, i.e., nanobody or VHH domain); and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR, which is adjacent to or in frame with one or more framework sequences. Tn antigen-binding fragments comprising a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) VH-CH1; (ii) VH-CH2; (iii) VH-CH3; (iv) VH-CH1-CH2; (v) VH-CH1-CH2-CH3, (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3, and (xiv) VL-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric VH or VL domain (e.g., by disulfide bond(s)).

As with full antibody molecules, antigen-binding fragments may be mono-specific or multi-specific (e.g., bi-specific). A multi-specific antigen-binding fragment of an antibody will typically comprise at least two different variable domains, wherein each variable domain is capable of specifically binding to a separate antigen or to a different epitope on the same antigen. Any multi-specific antibody format, including the exemplary bi-specific antibody formats disclosed herein, may be adapted for use in the context of an antigen-binding fragment of an antibody of the present invention using routine techniques available in the art.

In some preferred embodiments, an expression cassette is provided comprising nucleotide sequence encoding (i) aflibercept+anti-Ang2 HL scFab (ii) aflibercept+anti-Ang2 LH scFab (iii) aflibercept+anti-Ang2 HL scFv or (iv) aflibercept+anti-Ang2 LH scFv, preferably wherein the scFab or scFV comprises HCDR1, HCDR2 and HCDR3 of SEQ ID Nos: 52-54 and LCDR1, LCDR2 and LCDR3 of SEQ ID Nos: 55-57.

Nucleic Acids Encoding a First Anti-Angiogenic Polypeptide and a Soluble Fusion Protein

In some embodiments, a nucleic acid is provided comprising nucleotide sequence encoding a first and second anti-angiogenic polypeptide, wherein the first anti-angiogenic polypeptide is aflibercept and the second anti-angiogenic polypeptide a soluble fusion protein that inhibits the activity of a pro-angiogenic polypeptide.

In some preferred embodiments, the first anti-angiogenic polypeptide is aflibercept and the second anti-angiogenic polypeptide is a soluble form of a VEGF receptor (e.g., comprises one or more VEGF-binding domains of VEGFR-1, VEGFR-2 and/or VEGFR-3).

In some preferred embodiments, the first anti-angiogenic polypeptide is aflibercept and the second anti-angiogenic polypeptide is a soluble fusion protein comprising one or more VEGF-binding portions from the extracellular domain of VEGFR-3, representative examples of which include soluble fusion proteins as described in U.S. Pat. Nos. 7,034,105, 5,952,199, and 7,422,741, the contents of each of which is incorporated herein by reference.

VEGF-C promotes angiogenesis and lymphangiogenesis and increases vascular permeability and leakage. VEGF-C is elevated in the eyes of wAMD patients after anti-VEGF treatment. Delivery of VEGFR-3-FC, which binds VEGF-C and VEGF-D, in combination with aflibercept (which binds VEGF-A, VEGF-B and PIGF) provides an improved therapy for ocular disease such as wAMD and DME.

A combination of targeting VEGF-A with a blockade of VEGFR3 is effective in preclinical models of choroidal neovascularization (CNV) and is being evaluated in clinical trials. In a phase 2b clinical trial in subjects with neovascular AMD, the combination of intravitreal ranibizumab and VEGF C/D antagonist OPT-302 resulted in a benefit of 3.4 letters (p=0.0107) in mean best-corrected visual acuity at 24 weeks compared with treatment with ranibizumab alone.

In particularly preferred aspects, the second anti-angiogenic polypeptide is Opt-302, a soluble form of VEGFR-3 comprising the extracellular domains 1-3 of human VEGFR-3 and the Fc fragment of human IgG1 that binds and inhibits the activity of VEGF-C and VEGF-D on endogenous VEGFR-2 and VEGFR-3, described in U.S. Pat. No. 9,745,558, the contents of which are incorporated herein by reference.

A preferred nucleotide sequence encoding OPT-302, codon-optimized for expression in humans, is provided below:

(SEQ ID NO: 58)
ATGCAAAGAGGCGCCGCTCTCTGTCTGAGACTGTGGCTGTGTCTGGGCCTGCTGGAT
GGACTGGTGTCTGGCTACAGCATGACCCCTCCAACACTGAACATCACCGAGGAATC
CCACGTGATCGACACCGGCGATAGCCTGAGCATCAGCTGCAGAGGACAGCACCCTC
TGGAATGGGCTTGGCCTGGTGCTCAAGAAGCTCCTGCCACAGGCGACAAGGACAGC
GAGGATACAGGCGTTGTGCGGGATTGCGAGGGCACAGATGCCAGACCTTACTGCAA
GGTGCTGCTGCTGCACGAAGTGCACGCCCAGGATACCGGCAGCTACGTGTGCTACT
ACAAGTACATCAAGGCCCGGATCGAGGGCACCACAGCCGCAAGCTCTTATGTGTTC
GTGCGGGACTTCGAGCAGCCCTTCATCAACAAGCCCGACACACTGCTGGTCAACCG
GAAGGACGCTATGTGGGTGCCCTGTCTGGTGTCTATCCCCGGCCTGAATGTGACCCT
GAGAAGCCAGAGTTCCGTGCTGTGGCCTGATGGCCAAGAGGTCGTGTGGGACGATA
GAAGGGGCATGCTGGTGTCCACACCTCTGCTGCATGATGCCCTGTACCTGCAGTGCG
AGACAACCTGGGGCGACCAGGACTTCCTGAGCAACCCTTTCCTGGTGCACATCACCG
GCAACGAGCTGTACGACATCCAGCTGCTGCCTCGCAAGAGCCTGGAACTGCTCGTG
GGAGAGAAGCTGGTGCTGAACTGTACCGTGTGGGCCGAGTTCAATAGCGGCGTGAC
CTTCGACTGGGACTACCCTGGAAAGCAGGCCGAGCGTGGAAAATGGGTGCCCGAGA
GAAGAAGCCAGCAGACCCACACAGAGCTGAGCAGCATCCTGACCATCCACAACGTG
TCCCAGCACGATCTGGGCTCTTACGTGTGCAAGGCCAACAACGGCATCCAGCGGTTC
CGGGAAAGCACCGAAGTGATCGTGCATGAGGAACCCAAGAGCTGCGACAAGACAC
ACACCTGTCCTCCATGTCCTGCTCCAGAGCTTCTCGGCGGACCTTCCGTGTTCCTGTT
TCCTCCAAAGCCTAAGGACACCCTGATGATCAGCAGAACCCCTGAAGTGACCTGCG
TGGTGGTGGATGTGTCCCACGAGGATCCCGAAGTGAAGTTCAATTGGTACGTGGAC
GGCGTGGAAGTGCACAATGCCAAGACCAAGCCTAGAGAGGAACAGTACAACAGCA
CCTACAGAGTGGTGTCCGTGCTGACCGTGCTGCATCAGGATTGGCTGAACGGCAAA
GAGTACAAGTGCAAGGTGTCCAACAAGGCCCTGCCTGCTCCTATCGAGAAAACCAT
CTCCAAGGCCAAGGGCCAGCCTCGGGAACCTCAAGTGTATACCCTGCCTCCTAGCCG
CGACGAACTCACCAAGAATCAAGTGTCTCTGACATGTCTCGTGAAGGGGTTTTACCC
CAGCGACATTGCCGTCGAGTGGGAGTCCAATGGACAACCCGAGAACAATTATAAGA
CCACGCCACCAGTCCTGGACTCCGACGGCTCATTTTTTCTCTACTCCAAACTGACCGT
GGATAAGTCCCGGTGGCAGCAAGGGAATGTGTTTTCCTGTAGCGTGATGCATGAAG
CTCTCCACAATCATTACACCCAAAAATCTCTGTCTCTGAGCCCCGGCAAATGA

A preferred nucleotide sequence encoding a soluble VEGFR-3 containing an alternative IgG2 Fc domain, codon-optimized for expression in humans, is provided below:

(SEQ ID NO: 59)
ATGCAGAGGGGAGCCGCCCTGTGCCTGAGGCTGTGGCTGTGCCTGGGCCTGCTGGA
CGGCCTGGTGTCTGGCTACAGCATGACCCCCCCTACACTGAACATCACCGAGGAGA
GCCACGTGATCGACACAGGCGATAGCCTGTCCATCTCTTGCAGGGGCCAGCACCCCC
TGGAGTGGGCATGGCCTGGAGCACAGGAGGCACCAGCCACCGGCGACAAGGATAG
CGAGGACACAGGAGTGGTGCGGGACTGCGAGGGAACCGATGCCAGACCTTACTGTA
AGGTGCTGCTGCTGCACGAGGTGCACGCCCAGGATACAGGCTCCTACGTGTGCTACT
ATAAGTATATCAAGGCAAGGATCGAGGGAACCACAGCAGCCAGCTCCTACGTGTTC
GTGCGGGATTTTGAGCAGCCTTTCATCAACAAGCCAGACACCCTGCTGGTGAATCGG
AAGGATGCCATGTGGGTGCCCTGTCTGGTGTCTATCCCTGGCCTGAATGTGACACTG
AGAAGCCAGTCTAGCGTGCTGTGGCCAGACGGACAGGAGGTGGTGTGGGACGATCG
GAGAGGCATGCTGGTGAGCACCCCTCTGCTGCACGATGCCCTGTACCTGCAGTGCGA
GACAACATGGGGCGACCAGGATTTTCTGTCCAACCCTTTCCTGGTGCACATCACAGG
CAATGAGCTGTATGACATCCAGCTGCTGCCACGGAAGTCCCTGGAGCTGCTGGTGGG
CGAGAAGCTGGTGCTGAACTGTACCGTGTGGGCCGAGTTTAATTCTGGCGTGACATT
CGACTGGGATTACCCCGGCAAGCAGGCCGAGAGGGGCAAGTGGGTGCCTGAGAGG
CGCAGCCAGCAGACCCACACAGAGCTGTCCTCTATCCTGACCATCCACAACGTGAG
CCAGCACGATCTGGGCTCCTACGTGTGCAAGGCCAACAATGGCATCCAGCGGTTTA
GAGAGTCTACAGAAGTGATCGTGCACGAGGAGAGGAAGTGCTGCGTGGAGTGCCCA
CCATGTCCAGCACCTCCAGTGGCAGGACCATCCGTGTTCCTGTTTCCACCTAAGCCT
AAGGACACCCTGATGATCAGCCGCACCCCAGAGGTGACATGCGTGGTGGTGGACGT
GTCCCACGAGGATCCAGAGGTGCAGTTCAACTGGTACGTGGATGGCGTGGAGGTGC
ACAATGCCAAGACCAAGCCCAGGGAGGAGCAGTTTAATTCTACCTTCCGCGTGGTG
AGCGTGCTGACAGTGGTGCACCAGGACTGGCTGAACGGCAAGGAGTATAAGTGCAA
GGTGTCTAATAAGGGCCTGCCCGCCCCTATCGAGAAGACCATCAGCAAGACAAAGG
GACAGCCACGGGAGCCACAGGTGTACACCCTGCCACCATCCAGAGAGGAGATGACC
AAGAACCAGGTGTCTCTGACATGTCTGGTGAAGGGCTTTTATCCAAGCGACATCGCC
GTGGAGTGGGAGTCCAATGGCCAGCCCGAGAACAATTACAAGACCACACCTCCAAT
GCTGGACTCCGATGGCTCTTTCTTTCTGTATTCCAAGCTGACCGTGGATAAGTCTCGG
TGGCAGCAGGGCAACGTGTTCAGCTGTTCCGTGATGCACGAGGCCCTGCACAATCA
CTACACACAGAAGTCTCTGAGCCTGTCCCCCGGCAAGTGA

In some preferred embodiments, an expression cassette is provided comprising nucleotide sequence encoding (i) aflibercept+VEGFR3-Fc or (ii) aflibercept+VEGFR3-Fc-IgG2. Preferably, the nucleotide sequence encoding aflibercept comprises the sequence of SEQ ID NO:50 or a sequence at least 90%, identical thereto and/or the nucleotide sequence encoding VEGFR3-Fc comprises the sequence of SEQ ID NO:58 and/or the nucleotide sequence encoding VEGFR3-Fc-IgG2 comprises the sequences of SEQ ID NO:59.

Nucleic Acids Encoding a First and Second Anti-Angiogenic Polypeptide

In some embodiments, a nucleic acid is provided comprising nucleotide sequence encoding a first and second anti-angiogenic polypeptide, wherein the first anti-angiogenic polypeptide is aflibercept and the second anti-angiogenic polypeptide is selected from endostatin; tumstatin; angiostatin; pigment epithelium-derived factor (PEDF).

In some preferred embodiments, the nucleic acid comprises nucleotide sequence encoding aflibercept and nucleotide sequence encoding PEDF (see e.g., Dawson et al., Science 285:245, 1999; U.S. Pat. No. 5,840,686, and International Patent Applications WO93/24529 and WO99/04806, the contents of each of which is incorporated herein by reference). PEDF is a secreted protein with homology to members of the serpin family of serine protease inhibitors. PEDF is predominantly produced by retinal pigment epithelial cells, is expressed in most human tissues, and has anti-angiogenic and neuroprotectant qualities (see e.g., Dawson D W et al., Science. 1999 Jul. 9; 285(5425):245-8). PEDF prevents photoreceptor degeneration and deficiency of PEDF is associated with angiogenic diseases such as wAMD. Preclinical data points to an inhibitory role of VEGF and FGF in mouse and pig models of choroidal neovascularization (CNV) (see e.g., Lei X L, Oxid Med Cell Longev.; Vol. 2020, Art. ID 8941057).

A representative human PEDF sequence is found at GenBank Accession P36955 (e.g., P36955.4). In some aspects, a preferred nucleotide sequence encoding human PEDF that has been codon-optimized for expression in humans has the following sequence or a sequence at least 90%, at least 95%, at least 98% or at least 99% identical thereto:

(SEQ ID NO: 60)
ATGCAAGCTCTGGTGCTGCTGCTGTGTATCGGAGCCCTGCTGGGCCACAGCTCCTGT
CAAAATCCTGCCTCTCCACCTGAGGAAGGCAGCCCCGATCCAGATTCTACAGGCGCC
CTGGTGGAAGAAGAGGACCCATTCTTCAAGGTGCCCGTGAACAAACTGGCCGCTGC
CGTGTCCAACTTCGGCTACGACCTGTACAGAGTGCGGAGCAGCACAAGCCCCACCA
CCAATGTTCTGCTGAGCCCTCTGTCTGTGGCCACCGCTCTTTCTGCTCTGTCTCTGGG
AGCCGAGCAGAGAACCGAGAGCATCATTCACAGAGCCCTGTACTACGATCTGATCA
GCAGCCCTGACATCCACGGCACCTACAAAGAACTGCTGGACACCGTGACAGCCCCT
CAGAAGAATCTGAAGTCCGCCAGCCGGATCGTGTTCGAGAAGAAGCTGCGGATCAA
GAGCAGCTTCGTGGCCCCTCTGGAAAAGAGCTACGGCACCAGACCTAGAGTGCTGA
CCGGCAATCCCAGACTGGACCTGCAAGAGATCAACAACTGGGTGCAAGCCCAGATG
AAGGGCAAGCTGGCCAGAAGCACCAAAGAGATCCCCGACGAGATCAGCATCCTGCT
GCTGGGCGTCGCCCACTTTAAAGGCCAGTGGGTCACCAAGTTCGACTCCAGAAAGA
CCAGCCTCGAGGACTTCTACCTGGACGAGGAACGGACCGTCAGAGTGCCCATGATG
AGCGATCCTAAGGCCGTGCTGAGATACGGCCTGGATAGCGACCTGAGCTGCAAGAT
TGCTCAGCTGCCTCTGACCGGCTCTATGAGCATCATATTCTTTCTGCCCCTGAAAGTG
ACCCAGAATCTGACCCTGATCGAGGAAAGCCTGACCAGCGAGTTCATCCACGACAT
CGACCGCGAGCTGAAAACCGTGCAGGCTGTGCTGACTGTGCCCAAGCTGAAGCTGA
GCTACGAGGGCGAAGTGACCAAGAGCCTGCAAGAAATGAAGCTGCAGAGCCTGTTC
GACAGCCCCGACTTCAGCAAGATCACCGGCAAGCCCATCAAGCTGACCCAGGTGGA
ACACAGAGCCGGCTTCGAGTGGAATGAAGATGGCGCCGGAACCACACCTTCTCCAG
GACTGCAACCTGCTCACCTGACCTTTCCACTGGACTACCACCTGAACCAGCCTTTCA
TCTTCGTGCTGCGGGACACAGATACTGGCGCCCTGCTGTTCATCGGCAAGATCCTGG
ATCCTAGAGGCCCCTGA

In some preferred embodiments, an expression cassette is provided comprising nucleotide sequence encoding aflibercept+PEDF, preferably wherein the nucleotide sequence encoding aflibercept comprises the sequence of SEQ ID NO:50 and/or the nucleotide sequence encoding PEDF comprises the sequence of SEQ ID NO:60.

Nucleic Acids Encoding a First Anti-Angiogenic Polypeptide and an Interfering RNA Molecule that Reduces Expression of an Angiopoietin

In some embodiments, a nucleic acid is provided that comprises nucleotide sequence encoding a first anti-angiogenic polypeptide and an interfering RNA molecule that targets an angiopoietin, wherein the first anti-angiogenic polypeptide is aflibercept.

In preferred aspects, the interfering RNA molecule targets human angiopoietin-1 (aka Ang1 or Ang-1) and/or targets human angiopoietin-2 (aka Ang2 or Ang-2). Representative human Ang2 sequences can be found at e.g., NCBI Accession No. 015123 and SEQ ID Nos: 517 and 518 of U.S. Pat. No. 8,987,420, the contents of which are incorporated herein by reference. Suitable target sequences within the human Ang-2 gene as well as representative interfering RNA molecules targeting human Ang-2 include those in U.S. Pat. No. 7,994,305 (e.g., SEQ ID Nos:228-427 of U.S. Pat. No. 7,994,305) and U.S. Pat. No. 8,829,179 (e.g., SEQ ID Nos:2-69 and 73-104 of U.S. Pat. No. 8,829,179), the contents of each of which are incorporated herein by reference. In particularly preferred embodiments, the interfering RNA molecule targets human Ang-2 and comprises a sense strand and antisense strand according to Table 1.

In preferred embodiments, an expression cassette is provided comprising nucleotide sequence encoding aflibercept and one or more interfering RNAs set forth in Table 1. Preferably, the nucleotide sequence encoding aflibercept comprises the sequence of SEQ ID NO:50.

Nucleic Acids Encoding a First Anti-Angiogenic Polypeptide and an Interfering RNA Molecule that Reduces Expression of VEGF-C and/or VEGF-D

In some preferred embodiments, a nucleic acid is provided comprising nucleotide sequence encoding a first anti-angiogenic polypeptide and an interfering RNA molecule that targets VEGF-C and/or VEGF-D, wherein the first anti-angiogenic polypeptide is aflibercept.

In particularly preferred aspects, the interfering RNA molecule targets human VEGF-C and/or human VEGF-D. Representative human VEGF-C sequences can be found at e.g., GenBank Accession numbers NM 005429 and X94216. Representative human VEGF-D sequences can be found e.g., at GenBank Accession number AJ000185.1. Suitable target sequences within the human VEGF-C gene as well as representative interfering RNA molecules targeting human VEGF-C include those in U.S. Pat. No. 7,517,864 (e.g., Table 11) and US Patent Application Publication No. US2011/0293625 (e.g., SEQ ID Nos:1-3 and 7-12), the contents of each of which are incorporated herein by reference. In particularly preferred embodiments, the interfering RNA molecule targets human VEGF-C and comprises a sense strand and antisense strand according to Table 2.

VEGF-C target sequence(s) were selected based on in silico determination of specificity, homology to human and non-human primate (NHP) sequences and knockdown of VEGF-C in vitro (see working examples). The region of VEGF-C targeted by the RNAi molecule(s) has 100% homology between human and NHP sequences, whereas mouse VEGF-C has 2 point mutations that would likely influence the ability of this target sequence to be effective in mice. A sequence alignment of the VEGF-C target region is provided below:

VEGF-C miRNA Target Sequence
Homo sapiens
...CTACCTCAGCAAGACGTTATTT...
Macaca fascicularis
...CTACCTCAGCAAGACGTTATTT...
Chlorocebus aethiops
...CTACCTCAGCAAGACGTTATTT...
Mus musculus
...TTACCTCAGCAAGACGTTGTTT...

Wet AMD (wAMD) is a retinal condition characterized by growth of abnormal, leaky blood vessels from the choroidal layer through Bruch's membrane and into the retina which can lead to a rapid loss of central vision. Current approved treatments include injections of anti-angiogenic protein therapies, such as aflibercept, ranibizumab, or brolucizumab, or the aptamer pegaptinib sodium that block signaling through VEGF-A. However, these injected therapies require repeated intravitreal (IVT) administrations to maintain vision. Many patients fail to maintain initial visual benefit due to undertreatment related to burdensome frequency of required treatment visits. Nucleic acids described herein comprising an anti-angiogenic polypeptide targeting VEGF-A (e.g., aflibercept) and an RNAi molecule targeting VEG-C provides an improved efficacy for wetAMD patients by reducing expression of additional angiogenic factors such as VEGF-C that are upregulated following administration of current anti-VEGF therapies.

In preferred embodiments, an expression cassette is provided comprising nucleotide sequence encoding aflibercept and one or more interfering RNAs set forth in Table 2. Preferably, the nucleotide sequence encoding aflibercept comprises the sequence of SEQ ID NO:50.

Nucleic Acids Encoding a First Anti-Angiogenic Polypeptide and an Interfering RNA Molecule that Reduces Expression of VEGFR-3

In some embodiments, a nucleic acid is provided comprising nucleotide sequence encoding a first anti-angiogenic polypeptide and an interfering RNA molecule that targets VEGFR-3, wherein the first anti-angiogenic polypeptide is aflibercept.

In preferred aspects, the interfering RNA molecule targets human VEGFR-3. Representative human VEGFR-3 sequences can be found e.g., at GenBank Accession number X68203. Suitable target sequences within the human VEGFR-3 gene as well as representative interfering RNA molecules targeting human VEGFR-3 include those listed in Table II of U.S. Pat. No. 7,517,864, the contents of each of which are incorporated herein by reference. In particularly preferred embodiments, the interfering RNA molecule targets human VEGFR-3 and comprises a sense strand and antisense strand according to Table 3.

In preferred embodiments, an expression cassette is provided comprising nucleotide sequence encoding aflibercept and one or more interfering RNAs set forth in Table 3. Preferably, the nucleotide sequence encoding aflibercept comprises the sequence of SEQ TD NO:50.

In embodiments where the nucleic acid encodes a first anti-angiogenic polypeptide and one or more interfering RNAs, the sequence encoding the interfering RNA(s) may be placed within a natural or artificial intron (e.g., an artificial intron, within a transcription control sequence, within a 5′ UTR region of a gene, within the coding sequence of a gene or within the 3′ UTR region of a gene). In some aspects, the interfering RNA is placed within a synthetic U2 or U12 based intron or within an interferon regulating factor 7 intron 4 (IRF7int4; 93 bp).

In some preferred aspects, the interfering RNA is placed within an artificial intron in a transcription control sequence. In some preferred aspects, the intron is located within the hybrid chicken β-actin and rabbit β-globin intron of the CAG promoter, whereby the intron is co-transcribed within a pre-mRNA by Pol-II and cleaved out of the pre-mRNA by RNA splicing. The spliced intron containing the pre-miRNA structure is further processed into mature miRNA capable of silencing a pro-angiogenic target gene.

In other aspects, the interfering RNA is placed within an artificial intron that is located within the coding sequence of a gene (e.g., encoding aflibercept), whereby the intron is co-transcribed within a pre-mRNA by Pol-II and cleaved out of the pre-mRNA by RNA splicing. The spliced intron containing the pre-miRNA structure is further processed into mature miRNA capable of silencing a pro-angiogenic target gene.

In other aspects, the sequence encoding the interfering RNA is placed within the 5′ UTR or 3′UTR region of a gene but is not within an intron, in which case some portion (e.g., 50%) of the transcribed pre-mRNA is translated into the encoded protein and some portion (e.g., 50%) of the transcribed pre-mRNA is processed into active shRNA or miRNA. In some preferred aspects, the interfering RNA is placed within a 3′ UTR region of a gene.

Codon Optimized Nucleic Acid Sequences

In some embodiments, the present invention provides a nucleic acid molecule comprising a nucleotide sequence that is codon optimized for expression in humans. In some embodiments, the nucleotide sequence encodes PEDF and comprises or consists of the nucleotide sequence set forth as SEQ ID NO:60 or a sequence at least 90%, at least 95%, at least 98% or at least 99% identical thereto. In another embodiment, the nucleotide sequence encodes OPT-302 and comprises or consists of the nucleotide sequence set forth as SEQ ID NO:58 or a sequence at least 90%, at least 95%, at least 98% or at least 99% identical thereto. In another embodiment, the nucleotide sequence encodes VEGFR3-Fc-IgG2 and comprises or consists of the nucleotide sequence set forth as SEQ ID NO:59 or a sequence at least 90/o, at least 95%, at least 98% or at least 99% identical thereto.

The term “codon-optimized” as it refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA. Such optimization includes replacing at least one, or more than one, or a significant number, of codons with one or more codons that are more frequently used in the genes of that organism.

Deviations in the nucleotide sequence that comprises the codons encoding the amino acids of, any polypeptide chain allow for variations in the sequence coding for the gene. Since each codon consists of three nucleotides, and the nucleotides comprising DNA are restricted to four specific bases, there are 64 possible combinations of nucleotides, 61 of which encode amino acids (the remaining three codons encode signals ending translation). The “genetic code” which shows which codons encode which amino acids is reproduced herein as Table 1. As a result, many amino acids are designated by more than one codon. For example, the amino acids alanine and proline are coded for by four triplets, serine and arginine by six, whereas tryptophan and methionine are coded by just one triplet. This degeneracy allows for DNA base composition to vary over a wide range without altering the amino acid sequence of the proteins encoded by the DNA.

TABLE 4
The Standard Genetic Code

| | T | C | A | G |
|---|---|---|---|---|
| T | TTT Phe (F) | TCT Ser (S) | TAT Tyr (Y) | TGT Cys (C) |
| | TTC | TCC | TAC″ | TGC |
| | TTA Leu (L) | TCA | TAA Stop | TGA Stop |
| | TTG | TGG | TAG Stop | TGG Trp (W) |
| C | CTT Leu (L) | CCT Pro (P) | CAT His (H) | CGT Arg (R) |
| | CTC | CCC | CAC | CGC |
| | CTA | CCA | CAA Gln (Q) | CGA |
| | CTG | CCG | CAG | CGG |
| A | ATT Ile (I) | ACT Thr (T) | AAT Asn (N) | AGT Ser (S) |
| | ATC | ACC | AAC | AGC |
| | ATA | ACA | AAA Lys (K) | AGA Arg (R) |
| | ATG Met (M) | ACG | AAG | AGG |
| G | GTT (Val) V | GCT Ala (A) | GAT Asp (D) | GGT Gly (G) |
| | GTC | GCC | GAC | GGC |
| | GTA | GCA | GAA Glu (E) | GGA |
| | GTG | GCG | GAG | GGG |

Many organisms display a bias for use of particular codons to code for insertion of a particular amino acid in a growing peptide chain. Codon preference, or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

Given the large number of gene sequences available for a wide variety of animal, plant and microbial species, the relative frequencies of codon usage have been calculated. Codon usage tables are available, for example, at the “Codon Usage Database” available at www.kazusa.or.jp/codon/ (visited Jun. 18, 2012). See Nakamura, Y., et al. Nucl. Acids Res. 28:292 (2000).

Randomly assigning codons at an optimized frequency to encode a given polypeptide sequence can be done manually by calculating codon frequencies for each amino acid, and then assigning the codons to the polypeptide sequence randomly. Additionally, various algorithms and computer software programs can be used to calculate an optimal sequence.

Non-Viral Vectors

In some embodiments, a non-viral vector (e.g., an expression plasmid) is provided comprising any nucleotide sequence as herein described. In some embodiments, the non-viral vector comprises a nucleotide sequence encoding a first anti-angiogenic polypeptide (e.g., aflibercept) as herein described and one or more interfering RNA(s) as herein described and/or a second anti-angiogenic polypeptide as herein described. Preferably, the non-viral vector is a plasmid comprising an expression cassette comprising a nucleotide sequence as herein described.

Viral Vectors

In some embodiments, a viral vector comprising a modified (codon optimized) nucleic acid as herein described is provided. In preferred embodiments, the viral vector comprises a nucleic acid comprising nucleotide encoding a first anti-angiogenic polypeptide (e.g., aflibercept) and nucleotide sequence encoding one or more interfering RNA(s) as herein described and/or comprising nucleotide sequence encoding a first and second anti-angiogenic polypeptide. Examples of suitable viral vectors include but are not limited to adenoviral, retroviral, lentiviral, herpesvirus and adeno-associated virus (AAV) vectors.

In a preferred embodiment, the viral vector includes a portion of a parvovirus genome, such as an AAV genome with the rep and cap genes deleted and/or replaced by an expression cassette comprising sequence encoding a first anti-angiogenic polypeptide (e.g., aflibercept) and nucleotide sequence encoding one or more interfering RNA(s) as herein described and/or comprising nucleotide sequence encoding a first and second anti-angiogenic polypeptide and their associated expression control sequences. The expression cassette is typically inserted adjacent to one or two (i.e., is flanked by) AAV TRs or TR elements adequate for viral replication (Xiao et al., 1997, J. Virol. 71(2): 941-948), in place of the nucleic acid encoding viral rep and cap proteins. Other regulatory sequences suitable for use in facilitating tissue-specific expression in the target cell may also be included.

In some embodiments, the AAV viral vector comprises a nucleic acid comprising: (a) an AAV2 terminal repeat (b) a transcription control sequence (c) nucleotide sequence encoding a first anti-angiogenic polypeptide (d) nucleotide sequence(s) encoding an RNAi molecule as herein described (d) a polyadenylation sequence and (e) an AAV2 terminal repeat.

In other embodiments, the AAV viral vector comprises a nucleic acid comprising: (a) an AAV2 terminal repeat (b) a transcription control sequence (c) nucleotide sequence encoding a first anti-angiogenic polypeptide (d) a 2A sequence (e) nucleotide sequence encoding a second anti-angiogenic polypeptide (f) a polyadenylation sequence and (g) an AAV2 terminal repeat.

In particularly preferred embodiments, the AAV viral vector comprises a nucleic acid (transgene cassette) comprising the sequence of any of SEQ ID NOs:64-70, more preferably comprising the sequence of any of SEQ ID Nos: 68-70, or a sequence at least 90%, at least 95%, at least 98% or at least 99% identical thereto.

In some embodiments, the 5′ ITR has the following sequence:

(SEQ ID NO: 61)
TTGGCCACTCCCTCTCTGCGCGCTCGCTCGCTCACTGAGGCCGGGCGAC
CAAAGGTCGCCCGACGCCCGGGCTTTGCCCGGGCGGCCTCAGTGAGCGA
GCGAGCGCGCAGAGAGGGAGTGGCCAACTCCATCACTAGGGGTTCCT

In some embodiments, the 3′ ITR has the following sequence:

(SEQ ID NO: 62)
AGGAACCCCTAGTGATGGAGTTGGCCACTCCCTCTCTGCGCGCTCGCTC
GCTCACTGAGGCCGCCCGGGCAAAGCCCGGGCGTCGGGCGACCTTTGGT
CGCCCGGCCTCAGTGAGCGAGCGAGCGCGCAGAGAGGGAGTGGCCAA

In some embodiments, the SV40 polyadenylation sequence has the following sequence:

(SEQ ID NO: 63)
GGGGATCCAGACATGATAAGATACATTGATGAGTTTGGACAAACCACAA
CTAGAATGCAGTGAAAAAAATGCTTTATTTGTGAAATTTGTGATGCTAT
TGCTTTATTTGTAACCATTATAAGCTGCAATAAACAAGTTAACAACAAC
AATTGCATTCATTTTATGTTTCAGGTTCAGGGGGAGGTGTGGGAGGTTT
TTTAAAGCAAGTAAAACCTCTACAAATGTGGTATGGCTGATTATGATCA

Those skilled in the art will appreciate that an AAV vector comprising a transgene and lacking virus proteins needed for viral replication (e.g., cap and rep), cannot replicate since such proteins are necessary for virus replication and packaging. Helper viruses include, typically, adenovirus or herpes simplex virus. Alternatively, as discussed below, the helper functions (Ela, E1b, E2a, E4, and VA RNA) can be provided to a packaging cell including by transfecting the cell with one or more nucleic acids encoding the various helper elements and/or the cell can comprise the nucleic acid encoding the helper protein. For instance, HEK 293 were generated by transforming human cells with adenovirus 5 DNA and now express a number of adenoviral genes, including, but not limited to E1 and E3 (see, e.g., Graham et al., 1977, J. Gen. Virol. 36:59-72). Thus, those helper functions can be provided by the HEK 293 packaging cell without the need of supplying them to the cell by, e.g., a plasmid encoding them.

The viral vector may be any suitable nucleic acid construct, such as a DNA or RNA construct and may be single stranded, double stranded, or duplexed (i.e., self-complementary as described in WO 2001/92551).

The viral capsid component of the packaged viral vectors may be a parvovirus capsid. AAV Cap and chimeric capsids are preferred. For example, the viral capsid may be an AAV capsid (e.g., AAV1, AAV2, AAV3, AAV4, AAV5, AAV6, AAV7 AAV8, AAV9, AAV10, AAV11, AAV12, AAV1.1, AAV2.5, AAV6.1, AAV6.3.1, AAV9.45, AAVrh10, AAVrh74, RHM4-1, AAV2-TT, AAV2-TT-S312N, AAV3B-S312N, AAV-LK03, snake AAV, avian AAV, bovine AAV, canine AAV, equine AAV, ovine AAV, goat AAV, shrimp AAV, and any other AAV now known or later discovered. see, e.g., Fields et al., VIROLOGY, volume 2, chapter 69 (4.sup.th ed., Lippincott-Raven Publishers).

In some embodiments, the viral capsid component of the packaged viral vector is a variant of a native AAV capsid (i.e., comprises one or more modifications relative to a native AAV capsid). In some embodiments, the capsid is a variant of an AAV2, AAV5 or AAV8 capsid. In preferred embodiments, the capsid is a variant of an AAV2 capsid, such as those described in U.S. Patent Application Publication Number 2019/0255192A1 (e.g., comprising the amino acid sequence of any of SEQ ID NOs: 42-59), the entire contents of which are incorporated herein by reference. In a particularly preferred embodiment, the capsid comprises a VP1 capsid protein comprising, consisting essentially of, or consisting of the amino acid sequence of SEQ ID NO:48. In certain embodiments, the capsid protein comprises a peptide insertion in the Gi-loop of the capsid protein relative to a corresponding parental AAV capsid protein, wherein the peptide insertion comprises the amino acid sequence ISDQTKH (SEQ ID NO:74), preferably wherein the peptide insertion comprises the amino acid sequence Y₁Y₂ISDQTKHY₃(SEQ ID NO:75), wherein each of Y₁-Y₃ is independently selected from Ala, Leu, Gly, Ser, Thr, and Pro. In specific embodiments, the peptide insertion comprises the amino acid sequence LAISDQTKHA (SEQ ID NO:49), preferably wherein the insertion site is between amino acids corresponding to amino acids 587 and 588 of VP1 of AAV2 or the corresponding position in the capsid protein of another AAV serotype. In some embodiments, the capsid protein comprises one or more amino acid substitutions relative to VP1 capsid of AAV2 or one or more corresponding substitutions in another AAV serotype, preferably wherein the capsid protein comprises a P34A amino acid substitution relative to VP1 capsid of AAV2 or the corresponding substitution in another AAV serotype.

A full complement of AAV Cap proteins includes VP1, VP2, and VP3. The ORF comprising nucleotide sequences encoding AAV VP capsid proteins may comprise less than a full complement AAV Cap proteins or the full complement of AAV Cap proteins may be provided.

In yet another embodiment the present invention provides for the use of ancestral AAV vectors for use in therapeutic in vivo gene therapy. Specifically, in silico-derived sequences were synthesized de novo and characterized for biological activities. This effort led to the generation of nine functional putative ancestral AAVs and the identification of Anc80, the predicted ancestor of AAV serotypes 1, 2, 8 and 9 (Zinn et al., 2015, Cell Reports 12:1056-1068). Predicting and synthesis of such ancestral sequences in addition to assembling into a virus particle may be accomplished by using the methods described in WO 2015/054653, the contents of which are incorporated by reference herein. Notably, the use of the virus particles assembled from ancestral viral sequences may exhibit reduced susceptibility to pre-existing immunity in current day human population than do contemporary viruses or portions thereof.

The invention includes packaging cells, which are encompassed by “host cells,” which may be cultured to produce packaged viral vectors of the invention. The packaging cells of the invention generally include cells with heterologous (1) viral vector function(s), (2) packaging function(s), and (3) helper function(s). Each of these component functions is discussed in the ensuing sections.

Initially, the vectors can be made by several methods known to skilled artisans (see, e.g., WO 2013/063379). A preferred method is described in Grieger, et al. 2015, Molecular Therapy 24(2):287-297, the contents of which are incorporated by reference herein for all purposes. Briefly, efficient transfection of HEK293 cells is used as a starting point, wherein an adherent HEK293 cell line from a qualified clinical master cell bank is used to grow in animal component-free suspension conditions in shaker flasks and WAVE bioreactors that allow for rapid and scalable rAAV production. Using the triple transfection method (e.g., WO 96/40240), the suspension HEK293 cell line generates greater than 10⁵ vector genome containing particles (vg)/cell or greater than 10¹¹ vg/L of cell culture when harvested 48 hours post-transfection. More specifically, triple transfection refers to the fact that the packaging cell is transfected with three plasmids: one plasmid encodes the AAV rep and cap genes, another plasmid encodes various helper functions (e.g., adenovirus or HSV proteins such as Ela, E1 b, E2a, E4, and VA RNA, and another plasmid encodes the transgene and its various control elements (e.g., modified RPGRorf15 gene and hGRK promoter).

To achieve the desired yields, a number of variables are optimized such as selection of a compatible serum-free suspension media that supports both growth and transfection, selection of a transfection reagent, transfection conditions and cell density. A universal purification strategy, based on ion exchange chromatography methods, was also developed that resulted in high purity vector preps of AAV serotypes 1-6, 8, 9 and various chimeric capsids. This user-friendly process can be completed within one week, results in high full to empty particle ratios (>90% full particles), provides post-purification yields (>1×10¹¹ vg/L) and purity suitable for clinical applications and is universal with respect to all serotypes and chimeric particles. This scalable manufacturing technology has been utilized to manufacture GMP Phase I clinical AAV vectors for retinal neovascularization (AAV2), Hemophilia B (scAAV8), Giant Axonal Neuropathy (scAAV9) and Retinitis Pigmentosa (AAV2), which have been administered into patients. In addition, a minimum of a 5-fold increase in overall vector production by implementing a perfusion method that entails harvesting rAAV from the culture media at numerous time-points post-transfection.

The packaging cells include viral vector functions, along with packaging and vector functions. The viral vector functions typically include a portion of a parvovirus genome, such as an AAV genome, with rep and cap deleted and replaced by the first anti-angiogenic polypeptide sequence that inhibits the activity of VEGF-A and at least one synthetic RNA molecule or a second anti-angiogenic polypeptide sequence and its associated expression control sequences. The viral vector functions include sufficient expression control sequences to result in replication of the viral vector for packaging. Typically, the viral vector includes a portion of a parvovirus genome, such as an AAV genome with rep and cap deleted and replaced by the transgene and its associated expression control sequences. The transgene is typically flanked by two AAV TRs, in place of the deleted viral rep and cap ORFs. Appropriate expression control sequences are included, such as a tissue-specific promoter and other regulatory sequences suitable for use in facilitating tissue-specific expression of the transgene in the target cell. The transgene is typically a nucleic acid sequence that can be expressed to produce a therapeutic polypeptide or a marker polypeptide.

The terminal repeats (TR(s)) (resolvable and non-resolvable) selected for use in the viral vectors are preferably AAV sequences, with serotypes 1, 2, 3, 4, 5 and 6 being preferred. Resolvable AAV TRs need not have a wild-type TR sequence (e.g., a wild-type sequence may be altered by insertion, deletion, truncation or missense mutations), as long as the TR mediates the desired functions, e.g., virus packaging, integration, and/or provirus rescue, and the like. The TRs may be synthetic sequences that function as AAV inverted terminal repeats, such as the “double-D sequence” as described in U.S. Pat. No. 5,478,745 to Samulski et al., the entire disclosure of which is incorporated in its entirety herein by reference. Typically, but not necessarily, the TRs are from the same parvovirus, e.g., both TR sequences are from AAV2.

The packaging functions include capsid components. The capsid components are preferably from a parvoviral capsid, such as an AAV capsid or a chimeric AAV capsid function. Examples of suitable parvovirus viral capsid components are capsid components from the family Parvoviridae, such as an autonomous parvovirus or a Dependovirus. For example, the capsid components may be selected from AAV capsids, e.g., AAV1, AAV2, AAV3, AAV4, AAV5, AAV6, AAV7, AAV8, AAV9, AAV10, AAV11, AAV12, AAVrh10, AAVrh74, RHM4-1, RHM15-1, RHM15-2, RHM15-3/RHM15-5, RHM15-4, RHM15-6, AAV Hu.26, AAV1.1, AAV2.5, AAV6.1, AAV6.3.1, AAV9.45, AAV2i8, AAV2G9, AAV2i8G9, AAV2-TT, AAV2-TT-S312N, AAV3B-S312N, and AAV-LK03, and other novel capsids as yet unidentified or from non-human primate sources. Capsid components may include components from two or more AAV capsids.

The packaged viral vector generally includes sequence encoding one or more anti-angiogenic polypeptides and/or interfering RNAs as herein described and corresponding expression control sequence(s) flanked by TR elements, referred to herein as the “transgene” or “transgene expression cassette,” sufficient to result in packaging of the vector DNA and subsequent expression of the interfering RNA and/or gene sequence in the transduced cell (e.g., a photoreceptor). The viral vector functions may, for example, be supplied to the cell as a component of a plasmid or an amplicon. The viral vector functions may exist extrachromosomally within the cell line and/or may be integrated into the cell's chromosomal DNA.

Any method of introducing the nucleotide sequence carrying the viral vector functions into a cellular host for replication and packaging may be employed, including but not limited to, electroporation, calcium phosphate precipitation, microinjection, cationic or anionic liposomes, and liposomes in combination with a nuclear localization signal. In embodiments wherein the viral vector functions are provided by transfection using a virus vector; standard methods for producing viral infection may be used.

The packaging functions include genes for viral vector replication and packaging. Thus, for example, the packaging functions may include, as needed, functions necessary for viral gene expression, viral vector replication, rescue of the viral vector from the integrated state, viral gene expression, and packaging of the viral vector into a viral particle. The packaging functions may be supplied together or separately to the packaging cell using a genetic construct such as a plasmid or an amplicon, a Baculovirus, or HSV helper construct. The packaging functions may exist extrachromosomally within the packaging cell, but are preferably integrated into the cell's chromosomal DNA. Examples include genes encoding AAV Rep and Cap proteins.

The helper functions include helper virus elements needed for establishing active infection of the packaging cell, which is required to initiate packaging of the viral vector. Examples include functions derived from adenovirus, baculovirus and/or herpes virus sufficient to result in packaging of the viral vector. For example, adenovirus helper functions will typically include adenovirus components Ela, E1 b, E2a, E4, and VA RNA. The packaging functions may be supplied by infection of the packaging cell with the required virus. The packaging functions may be supplied together or separately to the packaging cell using a genetic construct such as a plasmid or an amplicon. See, e.g., pXR helper plasmids as described in Rabinowitz et al., 2002, J. Virol. 76:791, and pDG plasmids described in Grimm et al., 1998, Human Gene Therapy 9:2745-2760. The packaging functions may exist extrachromosomally within the packaging cell, but are preferably integrated into the cell's chromosomal DNA (e.g., E1 or E3 in HEK 293 cells).

Any suitable helper virus functions may be employed. For example, where the packaging cells are insect cells, baculovirus may serve as a helper virus. Herpes virus may also be used as a helper virus in AAV packaging methods. Hybrid herpes viruses encoding the AAV Rep protein(s) may advantageously facilitate for more scalable AAV vector production schemes.

Any method of introducing the nucleotide sequence carrying the helper functions into a cellular host for replication and packaging may be employed, including but not limited to, electroporation, calcium phosphate precipitation, microinjection, cationic or anionic liposomes, and liposomes in combination with a nuclear localization signal. embodiments wherein the helper functions are provided by transfection using a virus vector or infection using a helper virus; standard methods for producing viral infection may be used.

Any suitable permissive or packaging cell known in the art may be employed in the production of the packaged viral vector. Mammalian cells or insect cells are preferred. Examples of cells useful for the production of packaging cells in the practice of the invention include, for example, human cell lines, such as VERO, W138, MRC5, A549, HEK 293 cells (which express functional adenoviral E1 under the control of a constitutive promoter), B-50 or any other HeLa cells, HepG2, Saos-2, HuH7, and HT1080 cell lines. In one aspect, the packaging cell is capable of growing in suspension culture, more preferably, the cell is capable of growing in serum-free culture. In one embodiment, the packaging cell is a HEK293 that grows in suspension in serum free medium. In another embodiment, the packaging cell is the HEK293 cell described in U.S. Pat. No. 9,441,206 and deposited as ATCC No. PTA 13274. Numerous rAAV packaging cell lines are known in the art, including, but not limited to, those disclosed in WO 2002/46359. In another aspect, the packaging cell is cultured in the form of a cell stack (e.g., 10-layer cell stack seeded with HEK293 cells).

Cell lines for use as packaging cells include insect cell lines. Any insect cell which allows for replication of AAV and which can be maintained in culture can be used in accordance with the present invention. Examples include Spodoptera frugiperda, such as the Sf9 or Sf21 cell lines, Drosophila spp. cell lines, or mosquito cell lines, e.g., Aedes albopictus derived cell lines. A preferred cell line is the Spodoptera frugiperda Sf9 cell line. The following references are incorporated herein for their teachings concerning use of insect cells for expression of heterologous polypeptides, methods of introducing nucleic acids into such cells, and methods of maintaining such cells in culture: Methods in Molecular Biology, ed. Richard, Humana Press, N J (1995); O'Reilly et al., Baculovirus Expression Vectors: A Laboratory Manual, Oxford Univ. Press (1994); Samulski et al., 1989, J. Virol. 63:3822-3828; Kajigaya et al., 1991, Proc. Nat'l. Acad. Sci. USA 88: 4646-4650; Ruffing et al., 1992, J. Virol. 66:6922-6930; Kimbauer et al., 1996, Virol. 219:37-44; Zhao et al., 2000, Virol. 272:382-393; and Samulski et al., U.S. Pat. No. 6,204,059.

Virus capsids according to the invention can be produced using any method known in the art, e.g., by expression from a baculovirus (Brown et al., (1994) Virology 198:477-488). As a further alternative, the virus vectors of the invention can be produced in insect cells using baculovirus vectors to deliver the rep/cap genes and rAAV template as described, for example, by Urabe et al., 2002, Human Gene Therapy 13:1935-1943.

In another aspect, the present invention provides for a method of rAAV production in insect cells wherein a baculovirus packaging system or vectors may be constructed to carry the AAV Rep and Cap coding region by engineering these genes into the polyhedrin coding region of a baculovirus vector and producing viral recombinants by transfection into a host cell. Notably when using Baculovirus production for AAV, preferably the AAV DNA vector product is a self-complementary AAV like molecule without using mutation to the AAV ITR. This appears to be a by-product of inefficient AAV rep nicking in insect cells which results in a self-complementary DNA molecule by virtue of lack of functional Rep enzyme activity. The host cell is a baculovirus-infected cell or has introduced therein additional nucleic acid encoding baculovirus helper functions or includes these baculovirus helper functions therein. These baculovirus viruses can express the AAV components and subsequently facilitate the production of the capsids.

During production, the packaging cells generally include one or more viral vector functions along with helper functions and packaging functions sufficient to result in replication and packaging of the viral vector. These various functions may be supplied together or separately to the packaging cell using a genetic construct such as a plasmid or an amplicon, and they may exist extrachromosomally within the cell line or integrated into the cell's chromosomes.

The cells may be supplied with any one or more of the stated functions already incorporated, e.g., a cell line with one or more vector functions incorporated extrachromosomally or integrated into the cell's chromosomal DNA, a cell line with one or more packaging functions incorporated extrachromosomally or integrated into the cell's chromosomal DNA, or a cell line with helper functions incorporated extrachromosomally or integrated into the cell's chromosomal DNA

The rAAV vector may be purified by methods standard in the art such as by column chromatography or cesium chloride gradients. Methods for purifying rAAV vectors are known in the art and include methods described in Clark et al., 1999, Human Gene Therapy 10(6):1031-1039; Schenpp and Clark, 2002, Methods Mol. Med. 69:427-443; U.S. Pat. No. 6,566,118 and WO 98/09657.

Treatment Methods

In some embodiments, a nucleic acid as herein described—or a pharmaceutical composition comprising such a nucleic acid and a pharmaceutically acceptable excipient—is administered to a subject (e.g., a human) intraocularly, preferably by subretinal, suprachoroidal, or intravitreal injection. In some preferred embodiments, the nucleic acid or pharmaceutical composition is administered via intravitreal and/or subretinal injection, more preferably by a single intravitreal injection, to treat a VEGF-associated ocular disease. In some embodiments, the VEGF-associated ocular disease is a VEGF-A-associated ocular disease. In other embodiments, the nucleic acid or pharmaceutical composition is administered topically or intracamerally. In some embodiments, the VEGF-associated ocular disease is selected from wet (neovascular, exudative) age-related macular degeneration; macular edema following retinal vein occlusion; retinal neovascularization resulting from retinal vein occlusion; diabetic macular edema, diabetic retinopathy (including all stages of non-proliferative diabetic retinopathy and proliferative diabetic retinopathy), myopic macular degeneration, branch retinal vein occlusion, hemi-retinal vein occlusion, and central retinal vein occlusion; retinopathy of prematurity; idiopathic choroidal neovascularization; myopia macular degeneration and secondary retinal and choroidal neovascularization; retinal telangiectasia; neovascular glaucoma; vitreous hemorrhage; retinal and choroidal neovascularization secondary to retinal diseases, including but not limited to uveitis, trauma, retinal degenerative disorders, genetic retinal and/or choroidal disease, tumors of the eye, corneal and iris neovascularization. In preferred embodiments, the nucleic acid is delivered to the subject in a vector, preferably a recombinant AAV (rAAV) vector as herein described, preferably wherein the rAAV vector comprises a capsid protein of SEQ ID NO:48 or sequence comprising at least 90% identity thereto, or a pharmaceutical composition comprising such a vector and a pharmaceutically acceptable excipient.

In related aspects, a nucleic acid as herein described for use in the treatment of a VEGF-associated ocular disease (e.g., a VEGF-A-associated ocular disease) or for the manufacture of a medicament for the treatment of a VEGF-associated ocular disease is provided. In other related aspects, an rAAV comprising a nucleic acid as herein described for use in the treatment of a VEGF-associated ocular disease or for the manufacture of a medicament for the treatment of a VEGF-associated ocular disease is provided. In preferred embodiments, the rAAV comprises a capsid sequence of SEQ ID NO:48 or sequence comprising at least 90% identity thereto, and is intravitreally administered to a subject to treat a VEGF-associated ocular disease, preferably by a single intravitreal injection.

In certain preferred embodiments, a method is provided for the treatment and/or prevention of wet (neovascular, exudative) age-related macular degeneration; diabetic macular edema; macular edema following retinal vein occlusion; diabetic retinopathy; or myopic choroidal neovascularization and all other forms of abnormal ocular and retinal angiogenesis, including but not limited to idiopathic retinal neovascularization, neovascular glaucoma, retinopathy of prematurity, radiation retinopathy, central serous retinopathy, diabetic vitreous hemorrhage, pseudoxanthoma elasticum, Coat's and other forms of peripheral retinal neovasculation in a subject (e.g., human subject) by administering to the subject an effective amount of an rAAV comprising a nucleic acid as herein described or a pharmaceutical composition comprising such an rAAV and a pharmaceutically acceptable excipient. Preferably the rAAV comprises a capsid protein of SEQ ID NO:48 or sequence comprising at least 90% identity thereto. In particularly preferred embodiments, a method is provided for the treatment of wet age-related macular degeneration.

In some aspects, the nucleic acid comprises a nucleotide sequence encoding aflibercept and a nucleotide sequence encoding a second anti-angiogenic polypeptide. In related aspects, the second anti-angiogenic polypeptide is selected from endostatin; tumstatin; angiostatin; and pigment epithelium-derived factor (PEDF). In some preferred embodiments, the second anti-angiogenic polypeptide is PEDF. In particularly preferred embodiments, provided herein is an rAAV vector comprising a capsid protein of SEQ ID NO:48 and a nucleic acid comprising the following sequence (aflibercept+PEDF dual construct) or a sequence at least 80%, at least 85%, at least 90%, at least 95%; at least 98% or at least 99% identical thereto:

(SEQ ID NO: 64)
TTGGCCACTCCCTCTCTGCGCGCTCGCTCGCTCACTGAGGCCGGGCGACCAAAGGTC
GCCCGACCCCCGGGCTTTGCCCGGGCGGCCTCAGTGAGCGAGCGAGCGCGCAGAGA
GGGAGTGGCCAACTCCATCACTAGGGGTTCCTATCGATTGAATTCCAGGCGCGCCAT
CCTGCAGGTATTGACGTCAATAATGACGTATGTTCCCATAGTAACGCCAATAGGGAC
TTTCCATTGACGTCAATGGGTGGAGTATTTACGGTAAACTGCCCACTTGGCAGTACA
TCAAGTGTATCATATGCCAAGTACGCCCCCTATTGACGTCAATGACGGTAAATGGCC
CGCCTGGCATTATGCCCAGTACATGACCTTATGGGACTTTCCTACTTGGCAGTACAT
CTACGTATTAGTCATCGCTATTACCATGGTCGAGGTGAGCCCCACGTTCTGCTTCACT
CTCCCCATCTCCCCCCCCTCCCCACCCCCAATTTTGTATTTATTTATTTTTTAATTATT
TTGTGCAGCGATGGGGGGGGGGGGGGGGGGGGGGGGCGCGCCAGGCGGGGGGGGG
CGGGGCGAGGGGCGGGGGGGGGCGAGGCGGAGAGGTGCGGCGGCAGCCAATCAGA
GCGGCGCGCTCCGAAAGTTTCCTTTTATGGCGAGGCGGCGGCGGCGGCGGCCCTAT
AAAAAGCGAAGCGCGCGGGGGGCGGGAGTCGCTGCGCGCTGCCTTCGCCCCGTGCC
CCGCTCCGCCGCCGCCTCGCGCCGCCCGCCCCGGCTCTGACTGACCGCGTTACTCCC
ACAGGTGAGCGGGCGGGACGGCCCTTCTCCTCCGGGCTGTAATTAGCGCTTGGTTTA
ATGACGGCTTGTTTCTTTTCTGTGGCTGCGTGAAAGCCTTGAGGGGCTCCGGGAGGG
CCCTTTGTGCGGGGGGAGCGGCTCGGGGCTGTCCGCGGGGGGACGGCTGCCTTCGG
GGGGGACGGGGCAGGGGGGGGTTCGGCTTCTGGCGTGTGACCGGCGGCTCTAGAGC
CTCTGCTAACCATGTTCATGCCTTCTTCTTTTTCCTACAGCTCCTGGGCAACGTGCTG
GTTATTGTGCTGTCTCATCATTTTGGCAAAGAATTGTACAGGATATCTTGCTAGCACG
CCACCATGGTTTCTTACTGGGACACCGGCGTGCTGCTGTGTGCCCTGCTTTCTTGTCT
GCTGCTGACCGGCTCTAGCAGCGGCTCTGATACCGGCAGACCCTTCGTGGAAATGTA
CAGCGAGATCCCCGAGATCATCCACATGACCGAGGGCAGAGAGCTGGTCATCCCTT
GCAGAGTGACAAGCCCCAACATCACCGTGACTCTGAAGAAGTTCCCTCTGGACACA
CTGATCCCCGACGGCAAGAGAATCATCTGGGACAGCCGGAAGGGCTTCATCATCAG
CAACGCCACCTACAAAGAGATCGGCCTGCTGACCTGTGAAGCCACCGTGAATGGCC
ACCTGTACAAGACCAACTACCTGACACACAGACAGACCAACACCATCATCGACGTG
GTGCTGAGCCCTAGCCACGGCATTGAACTGTCTGTGGGCGAGAAGCTGGTGCTGAA
CTGTACCGCCAGAACCGAGCTGAACGTGGGCATCGACTTCAACTGGGAGTACCCCA
GCAGCAAGCACCAGCACAAGAAACTGGTCAACCGGGACCTGAAAACCCAGAGCGG
CAGCGAGATGAAGAAATTCCTGAGCACCCTGACCATCGACGGCGTGACCAGAAGTG
ACCAGGGCCTGTACACATGTGCCGCCAGCTCTGGCCTGATGACCAAGAAAAACAGC
ACCTTCGTGCGGGTGCACGAGAAGGACAAGACCCACACCTGTCCTCCATGTCCTGCT
CCAGAACTGCTCGGCGGACCTTCCGTGTTCCTGTTTCCTCCAAAGCCTAAGGACACC
CTGATGATCAGCAGAACCCCTGAAGTGACCTGCGTGGTGGTGGATGTGTCCCACGA
GGATCCCGAAGTGAAGTTCAATTGGTACGTGGACGGCGTGGAAGTGCACAACGCCA
AGACCAAGCCTAGAGAGGAACAGTACAATAGCACCTACAGAGTGGTGTCCGTGCTG
ACCGTGCTGCACCAGGATTGGCTGAACGGCAAAGAGTACAAGTGCAAGGTGTCCAA
CAAGGCCCTGCCTGCTCCTATCGAGAAAACCATCTCCAAGGCCAAGGGCCAGCCTA
GGGAACCCCAGGTTTACACACTGCCTCCAAGCAGGGACGAGCTGACAAAGAACCAG
GTGTCCCTGACCTGCCTGGTCAAGGGCTTCTACCCTTCCGATATCGCCGTGGAATGG
GAGAGCAATGGCCAGCCTGAGAACAACTACAAGACAACCCCTCCTGTGCTGGACAG
CGACGGCTCATTCTTCCTGTACAGCAAGCTGACAGTGGACAAGAGCAGATGGCAGC
AGGGCAACGTGTTCAGCTGCTCCGTGATGCACGAGGCCCTGCACAACCACTACACC
CAGAAGTCCCTGAGCCTGTCTCCTGGCAAACGGAAGAGAAGAGGCAGCGGCGAAG
GCAGAGGATCCCTGCTTACATGTGGCGACGTGGAAGAGAACCCCGGACCTATGCAA
GCTCTGGTGCTGCTGCTGTGTATCGGAGCCCTGCTGGGCCACAGCTCCTGTCAAAAT
CCTGCCTCTCCACCTGAGGAAGGCAGCCCCGATCCAGATTCTACAGGCGCCCTGGTG
GAAGAAGAGGACCCATTCTTCAAGGTGCCCGTGAACAAACTGGCCGCTGCCGTGTC
CAACTTCGGCTACGACCTGTACAGAGTGCGGAGCAGCACAAGCCCCACCACCAATG
TTCTGCTGAGCCCTCTGTCTGTGGCCACCGCTCTTTCTGCTCTGTCTCTGGGAGCCGA
GCAGAGAACCGAGAGCATCATTCACAGAGCCCTGTACTACGATCTGATCAGCAGCC
CTGACATCCACGGCACCTACAAAGAACTGCTGGACACCGTGACAGCCCCTCAGAAG
AATCTGAAGTCCGCCAGCCGGATCGTGTTCGAGAAGAAGCTGCGGATCAAGAGCAG
CTTCGTGGCCCCTCTGGAAAAGAGCTACGGCACCAGACCTAGAGTGCTGACCGGCA
ATCCCAGACTGGACCTGCAAGAGATCAACAACTGGGTGCAAGCCCAGATGAAGGGC
AAGCTGGCCAGAAGCACCAAAGAGATCCCCGACGAGATCAGCATCCTGCTGCTGGG
CGTCGCCCACTTTAAAGGCCAGTGGGTCACCAAGTTCGACTCCAGAAAGACCAGCC
TCGAGGACTTCTACCTGGACGAGGAACGGACCGTCAGAGTGCCCATGATGAGCGAT
CCTAAGGCCGTGCTGAGATACGGCCTGGATAGCGACCTGAGCTGCAAGATTGCTCA
GCTGCCTCTGACCGGCTCTATGAGCATCATATTCTTTCTGCCCCTGAAAGTGACCCA
GAATCTGACCCTGATCGAGGAAAGCCTGACCAGCGAGTTCATCCACGACATCGACC
GCGAGCTGAAAACCGTGCAGGCTGTGCTGACTGTGCCCAAGCTGAAGCTGAGCTAC
GAGGGCGAAGTGACCAAGAGCCTGCAAGAAATGAAGCTGCAGAGCCTGTTCGACAG
CCCCGACTTCAGCAAGATCACCGGCAAGCCCATCAAGCTGACCCAGGTGGAACACA
GAGCCGGCTTCGAGTGGAATGAAGATGGCGCCGGAACCACACCTTCTCCAGGACTG
CAACCTGCTCACCTGACCTTTCCACTGGACTACCACCTGAACCAGCCTTTCATCTTCG
TGCTGCGGGACACAGATACTGGCGCCCTGCTGTTCATCGGCAAGATCCTGGATCCTA
GAGGCCCCTGAGCCACGCGTAACACGTGCATGCGAGAGATCTGCGGCCGCGAGCTC
GGGGATCCAGACATGATAAGATACATTGATGAGTTTGGACAAACCACAACTAGAAT
GCAGTGAAAAAAATGCTTTATTTGTGAAATTTGTGATGCTATTGCTTTATTTGTAACC
ATTATAAGCTGCAATAAACAAGTTAACAACAACAATTGCATTCATTTTATGTTTCAG
GTTCAGGGGGAGGTGTGGGAGGTTTTTTAAAGCAAGTAAAACCTCTACAAATGTGG
TATGGCTGATTATGATCAATGCATCCTAGCCGGAGGAACCCCTAGTGATGGAGTTGG
CCACTCCCTCTCTGCGCGCTCGCTCGCTCACTGAGGCCGCCCGGGCAAAGCCCGGGC
GTCGGGCGACCTTTGGTCGCCCGGCCTCAGTGAGCGAGCGAGCGCGCAGAGAGGGA
GTGGCCAA,

and its use in treating wet (neovascular, exudative) age-related macular degeneration; diabetic macular edema; macular edema following retinal vein occlusion; diabetic retinopathy; or myopic choroidal neovascularization, preferably wherein the vector is intraocularly administered to a human subject, preferably wherein intraocular administration comprises intravitreal injection (e.g., a single intravitreal injection), subretinal injection or suprachoroidal injection.

In other aspects, the nucleic acid comprises a nucleotide sequence encoding aflibercept and a nucleotide sequence encoding a soluble fusion protein comprising one or more VEGF-binding portions from the extracellular domain of VEGFR-3. In particularly preferred embodiments, provided herein is an rAAV vector comprising a capsid protein of SEQ ID NO:48 and a nucleic acid comprising the following sequence (aflibercept+OPT-302 dual construct) or a sequence at least 80%, at least 85%, at least 90%, at least 95%; at least 98% or at least 99% identical thereto:

(SEQ ID NO: 65)
TTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGAGA
GGGAGTGGCCAACTCCATCACTAGGGGTTCCTATCGATTGAATTCCAGGCGCGCCAT
CCTGCAGGTATTGACGTCAATAATGACGTATGTTCCCATAGTAACGCCAATAGGGAC
TTTCCATTGACGTCAATGGGTGGAGTATTTACGGTAAACTGCCCACTTGGCAGTACA
TCAAGTGTATCATATGCCAAGTACGCCCCCTATTGACGTCAATGACGGTAAATGGCC
CGCCTGGCATTATGCCCAGTACATGACCTTATGGGACTTTCCTACTTGGCAGTACAT
CTACGTATTAGTCATCGCTATTACCATGGTCGAGGTGAGCCCCACCTTCTGCTTCACT
CTCCCCATCTCCCCCCCCTCCCCACCCCCAATTTTGTATTTATTTATTTTTTAATTATT
TTGTGCAGCGATGGGGGGGGGGGGGGGGGGGGGGGGCGCGCCAGGCGGGGGGGG
CGGGGCGAGGGGCGGGGGGGGGCGAGGCGGAGAGGTGCGGCGGCAGCCAATCAGA
GCGGCGCGCTCCGAAAGTTTCCTTTTATGGCGAGGCGGCGGCGGCGGCGGCCCTAT
AAAAAGCGAAGCGCGCGGCGGGGGGGAGTCGCTGCGCGCTGCCTTCGCCCCGTGCC
CCGCTCCGCCGCCGCCTCGCGCCGCCCCCCCCGGCTCTGACTGACCGCGTTACTCCC
ACAGGTGAGCGGGCGGGACGGCCCTTCTCCTCCGGGCTGTAATTAGCGCTTGGTTTA
ATGACGGCTTGTTTCTTTTCTGTGGCTGCGTGAAAGCCTTGAGGGGCTCCGGGAGGG
CCCTTTGTGCGGGGGGAGCGGCTCGGGGCTGTCCGCGGGGGGACGGCTGCCTTCGG
GGGGGACGGGGCAGGGGGGGGTTCGGCTTCTGGCGTGTGACCGGCGGCTCTAGAGC
CTCTGCTAACCATGTTCATGCCTTCTTCTTTTTCCTACAGCTCCTGGGCAACGTGCTG
GTTATTGTGCTGTCTCATCATTTTGGCAAAGAATTGTACAGGATATCTTGCTAGCACG
CCACCATGGTTTCTTACTGGGACACCGGCGTGCTGCTGTGTGCCCTGCTTTCTTGTCT
GCTGCTGACCGGCTCTAGCAGCGGCTCTGATACCGGCAGACCCTTCGTGGAAATGTA
CAGCGAGATCCCCGAGATCATCCACATGACCGAGGGCAGAGAGCTGGTCATCCCTT
GCAGAGTGACAAGCCCCAACATCACCGTGACTCTGAAGAAGTTCCCTCTGGACACA
CTGATCCCCGACGGCAAGAGAATCATCTGGGACAGCCGGAAGGGCTTCATCATCAG
CAACGCCACCTACAAAGAGATCGGCCTGCTGACCTGTGAAGCCACCGTGAATGGCC
ACCTGTACAAGACCAACTACCTGACACACAGACAGACCAACACCATCATCGACGTG
GTGCTGAGCCCTAGCCACGGCATTGAACTGTCTGTGGGCGAGAAGCTGGTGCTGAA
CTGTACCGCCAGAACCGAGCTGAACGTGGGCATCGACTTCAACTGGGAGTACCCCA
GCAGCAAGCACCAGCACAAGAAACTGGTCAACCGGGACCTGAAAACCCAGAGCGG
CAGCGAGATGAAGAAATTCCTGAGCACCCTGACCATCGACGGCGTGACCAGAAGTG
ACCAGGGCCTGTACACATGTGCCGCCAGCTCTGGCCTGATGACCAAGAAAAACAGC
ACCTTCGTGCGGGTGCACGAGAAGGACAAGACCCACACCTGTCCTCCATGTCCTGCT
CCAGAACTGCTCGGCGGACCTTCCGTGTTCCTGTTTCCTCCAAAGCCTAAGGACACC
CTGATGATCAGCAGAACCCCTGAAGTGACCTGCGTGGTGGTGGATGTGTCCCACGA
GGATCCCGAAGTGAAGTTCAATTGGTACGTGGACGGCGTGGAAGTGCACAACGCCA
AGACCAAGCCTAGAGAGGAACAGTACAATAGCACCTACAGAGTGGTGTCCGTGCTG
ACCGTGCTGCACCAGGATTGGCTGAACGGCAAAGAGTACAAGTGCAAGGTGTCCAA
CAAGGCCCTGCCTGCTCCTATCGAGAAAACCATCTCCAAGGCCAAGGGCCAGCCTA
GGGAACCCCAGGTTTACACACTGCCTCCAAGCAGGGACGAGCTGACAAAGAACCAG
GTGTCCCTGACCTGCCTGGTCAAGGGCTTCTACCCTTCCGATATCGCCGTGGAATGG
GAGAGCAATGGCCAGCCTGAGAACAACTACAAGACAACCCCTCCTGTGCTGGACAG
CGACGGCTCATTCTTCCTGTACAGCAAGCTGACAGTGGACAAGAGCAGATGGCAGC
AGGGCAACGTGTTCAGCTGCTCCGTGATGCACGAGGCCCTGCACAACCACTACACC
CAGAAGTCCCTGAGCCTGTCTCCTGGCAAACGGAAGAGAAGAGGCAGCGGCGAAG
GCAGAGGATCCCTGCTTACATGTGGCGACGTGGAAGAGAACCCCGGACCTATGCAA
AGAGGCGCCGCTCTCTGTCTGAGACTGTGGCTGTGTCTGGGCCTGCTGGATGGACTG
GTGTCTGGCTACAGCATGACCCCTCCAACACTGAACATCACCGAGGAATCCCACGTG
ATCGACACCGGCGATAGCCTGAGCATCAGCTGCAGAGGACAGCACCCTCTGGAATG
GGCTTGGCCTGGTGCTCAAGAAGCTCCTGCCACAGGCGACAAGGACAGCGAGGATA
CAGGCGTTGTGCGGGATTGCGAGGGCACAGATGCCAGACCTTACTGCAAGGTGCTG
CTGCTGCACGAAGTGCACGCCCAGGATACCGGCAGCTACGTGTGCTACTACAAGTA
CATCAAGGCCCGGATCGAGGGCACCACAGCCGCTAGCTCTTATGTGTTCGTGCGGG
ACTTCGAGCAGCCCTTCATCAACAAGCCCGACACACTGCTGGTCAACCGGAAGGAC
GCTATGTGGGTGCCCTGTCTGGTGTCTATCCCCGGCCTGAATGTGACCCTGAGAAGC
CAGAGTTCCGTGCTGTGGCCTGATGGCCAAGAGGTCGTGTGGGACGATAGAAGGGG
CATGCTGGTGTCCACACCTCTGCTGCATGATGCCCTGTACCTGCAGTGCGAGACAAC
CTGGGGCGACCAGGACTTCCTGAGCAACCCTTTCCTGGTGCACATCACCGGCAACGA
GCTGTACGACATCCAGCTGCTGCCTCGCAAGAGCCTGGAACTGCTCGTGGGAGAGA
ACCTGGTGCTGAACTGTACCGTGTGGGCCGAGTTCAATAGCGGCGTGACCTTCGACT
GGGACTACCCTGGAAAGCAGGCCGAGCGTGGAAAATGGGTGCCCGAGAGAAGAAG
CCAGCAGACCCACACAGAGCTGAGCAGCATCCTGACCATCCACAACGTGTCCCAGC
ACGATCTGGGCTCTTACGTGTGCAAGGCCAACAACGGCATCCAGCGGTTCCGGGAA
AGCACCGAAGTGATCGTGCATGAGGAACCCAAGAGCTGCGACAAGACACACACCTG
TCCTCCATGTCCTGCTCCAGAGCTTCTCGGCGGACCTTCCGTGTTCCTGTTTCCTCCA
AAGCCTAAGGACACCCTGATGATCAGCAGAACCCCTGAAGTGACCTGCGTGGTGGT
GGATGTGTCCCACGAGGATCCCGAAGTGAAGTTCAATTGGTACGTGGACGGCGTGG
AAGTGCACAATGCCAAGACCAAGCCTAGAGAGGAACAGTACAACAGCACCTACAG
AGTGGTGTCCGTGCTGACCGTGCTGCATCAGGATTGGCTGAACGGCAAAGAGTACA
AGTGCAAGGTGTCCAACAAGGCCCTGCCTGCTCCTATCGAGAAAACCATCTCCAAG
GCCAAGGGCCAGCCTCGGGAACCTCAAGTGTATACCCTGCCTCCTAGCCGCGACGA
ACTCACCAAGAATCAAGTGTCTCTGACATGTCTCGTGAAGGGGTTTTACCCCAGCGA
CATTGCCGTCGAGTGGGAGTCCAATGGACAACCCGAGAACAATTATAAGACCACGC
CACCAGTCCTGGACTCCGACGGCTCATTTTTTCTCTACTCCAAACTGACCGTGGATA
AGTCCCGGTGGCAGCAAGGGAATGTGTTTTCCTGTAGCGTGATGCATGAAGCTCTCC
ACAATCATTACACCCAAAAATCTCTGTCTCTGAGCCCCGGCAAATGAGCCACGCGTA
ACACGTGCATGCGAGAGATCTGCGGCCGCGAGCTCGGGGATCCAGACATGATAAGA
TACATTGATGAGTTTGGACAAACCACAACTAGAATGCAGTGAAAAAAATGCTTTATT
TGTGAAATTTGTGATGCTATTGCTTTATTTGTAACCATTATAAGCTGCAATAAACAA
GTTAACAACAACAATTGCATTCATTTTATGTTTCAGGTTCAGGGGGAGGTGTGGGAG
GTTTTTTAAAGCAAGTAAAACCTCTACAAATGTGGTATGGCTGATTATGATCAATGC
ATCCTAGCCGGAGGAACCCCTAGTGATGGAGTTGGCCACTCCCTCTCTGCGCGCTCG
CTCGCTCACTGAGGCCGCCCGGGCAAAGCCCGGGCGTCGGGCGACCTTTGGTCGCC
CGGCCTCAGTGAGCGAGCGAGCGCGCAGAGAGGGAGTGGCCAA

and its use in treating wet (neovascular, exudative) age-related macular degeneration; diabetic macular edema; macular edema following retinal vein occlusion; diabetic retinopathy; or myopic choroidal neovascularization, preferably wherein the vector is intraocularly administered to a human subject, preferably wherein intraocular administration comprises intravitreal injection (e.g., a single intravitreal injection), subretinal injection or suprachoroidal injection.

In other aspects, the nucleic acid comprises a nucleotide sequence encoding aflibercept and a nucleotide sequence encoding an antibody or antigen-binding fragment thereof that binds to and inhibits the activity of a pro-angiogenic protein. In preferred embodiments, the antibody or antigen-binding fragment thereof binds to human ang-1 or human ang-2. In particularly preferred embodiments, provided herein is an rAAV vector comprising a capsid protein of SEQ ID NO:48 and a nucleic acid comprising the following sequence (aflibercept+anti-Ang-2 HL construct) or a sequence at least 80%, at least 85%, at least 90%, at least 95%; at least 98% or at least 99% identical thereto:

(SEQ ID NO: 66)
TTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGAGA
GGGAGTGGCCAACTCCATCACTAGGGGTTCCTATCGATTGAATTGCAGGCGCGCCAT
CCTGCAGGTATTGACGTCAATAATGACGTATGTTCCCATAGTAACGCCAATAGGGAC
TTTCCATTGACGTCAATGGGTGGAGTATTTACGGTAAACTGCCCACTTGGCAGTACA
TCAAGTGTATCATATGCCAAGTACGCCCCCTATTGACGTCAATGACGGTAAATGGCC
CGCCTGGCATTATGCCCAGTACATGACCTTATGGGACTTTCCTACTTGGCAGTACAT
CTACGTATTAGTCATCGCTATTACCATGGTCGAGGTGAGCCCCACGTTCTGCTTCACT
CTCCCCATCTCCCCCCCCTCCCCACCCCCAATTTTGTATTTATTTATTTTTTAATTATT
TTGTGCAGCGATGGGGGGGGGGGGGGGGGGGGGGGGCGCGCCAGGCGGGGGGGGG
CGGGGCGAGGGGGGGGGGGGGGCGAGGCGGAGAGGTGCGGCGGCAGCCAATCAGA
GCGGCGCGCTCCGAAAGTTTCCTTTTATGGCGAGGCGGCGGCGGCGGCGGCCCTAT
AAAAAGCGAAGCGCGCGGGGGGCGGGAGTCGCTGCGCGCTGCCTTCGCCCCGTGCC
CCGCTCCGCCGCCGCCTCGCGCCGCCCGCCCCGGCTCTGACTGACCGCGTTACTCCC
ACAGGTGAGCGGGCGGGACGGCCCTTCTCCTCCGGGCTGTAATTAGCGCTTGGTTTA
ATGACGGCTTGTTTCTTTTCTGTGGCTGCGTGAAAGCCTTGAGGGGCTCCGGGAGGG
CCCTTTGTGCGGGGGGAGCGGCTCGGGGCTGTCCGCGGGGGGACGGCTGCCTTCGG
GGGGGACGGGGCAGGGGGGGGTTCGGCTTCTGGCGTGTGACCGGCGGCTCTAGAGC
CTCTGCTAACCATGTTCATGCCTTCTTCTTTTTCCTACAGCTCCTGGGCAACGTGCTG
GTTATTGTGCTGTCTCATCATTTTGGCAAAGAATTGTACAGGATATCTTGCTAGCACG
CCACCATGGTTTCTTACTGGGACACCGGCGTGCTGCTGTGTGCCCTGCTTTCTTGTCT
GCTGCTGACCGGCTCTAGCAGCGGCTCTGATACCGGCAGACCCTTCGTGGAAATGTA
CAGCGAGATCCCCGAGATCATCCACATGACCGAGGGCAGAGAGCTGGTCATCCCTT
GCAGAGTGACAAGCCCCAACATCACCGTGACTCTGAAGAAGTTCCCTCTGGACACA
CTGATCCCCGACGGCAAGAGAATCATCTGGGACAGCCGGAAGGGCTTCATCATCAG
CAACGCCACCTACAAAGAGATCGGCCTGCTGACCTGTGAAGCCACCGTGAATGGCC
ACCTGTACAAGACCAACTACCTGACACACAGACAGACCAACACCATCATCGACGTG
GTGCTGAGCCCTAGCCACGGCATTGAACTGTCTGTGGGCGAGAAGCTGGTGCTGAA
CTGTACCGCCAGAACCGAGCTGAACGTGGGCATCGACTTCAACTGGGAGTACCCCA
GCAGCAAGCACCAGCACAAGAAACTGGTCAACCGGGACCTGAAAACCCAGAGCGG
CAGCGAGATGAAGAAATTCCTGAGCACCCTGACCATCGACGGCGTGACCAGAAGTG
ACCAGGGCCTGTACACATGTGCCGCCAGCTCTGGCCTGATGACCAAGAAAAACAGC
ACCTTCGTGCGGGTGCACGAGAAGGACAAGACCCACACCTGTCCTCCATGTCCTGCT
CCAGAACTGCTCGGCGGACCTTCCGTGTTCCTGTTTCCTCCAAAGCCTAAGGACACC
CTGATGATCAGCAGAACCCCTGAAGTGACCTGCGTGGTGGTGGATGTGTCCCACGA
GGATCCCGAAGTGAAGTTCAATTGGTACGTGGACGGCGTGGAAGTGCACAACGCCA
AGACCAAGCCTAGAGAGGAACAGTACAATAGCACCTACAGAGTGGTGTCCGTGCTG
ACCGTGCTGCACCAGGATTGGCTGAACGGCAAAGAGTACAAGTGCAAGGTGTCCAA
CAAGGCCCTGCCTGCTCCTATCGAGAAAACCATCTCCAAGGCCAAGGGCCAGCCTA
GGGAACCCCAGGTTTACACACTGCCTCCAAGCAGGGACGAGCTGACAAAGAACCAG
GTGTCCCTGACCTGCCTGGTCAAGGGCTTCTACCCTTCCGATATCGCCGTGGAATGG
GAGAGCAATGGCCAGCCTGAGAACAACTACAAGACAACCCCTCCTGTGCTGGACAG
CGACGGCTCATTCTTCCTGTACAGCAAGCTGACAGTGGACAAGAGCAGATGGCAGC
AGGGCAACGTGTTCAGCTGCTCCGTGATGCACGAGGCCCTGCACAACCACTACACC
CAGAAGTCCCTGAGCCTGTCTCCTGGCAAACGGAAGAGAAGAGGCAGCGGCGAAG
GCAGAGGATCCCTGCTTACATGTGGCGACGTGGAAGAGAACCCCGGACCTATGGAT
TGGACCTGGTCCATCCTGTTTCTGGTGGCCGCTGCCACAGGCACATACTCTCAGGTT
CAGCTGGTGCAGTCTGGCGCCGAAGTGAAAAAACCTGGCGCCTCCGTGAAGGTGTC
CTGCAAGGCTAGCGGCTACACCTTTACCGGCTACTACATGCACTGGGTCCGACAGGC
TCCAGGACAGGGACTTGAATGGATGGGCTGGATCAACCCCAATAGCGGCGGCACCA
ATTACGCCCAGAAATTCCAGGGCAGAGTGACCATGACCAGAGACACCAGCATCAGC
ACCGCCTACATGGAACTGAGCCGGCTGAGATCCGATGACACCGCCGTGTACTACTG
CGCCAGATCTCCCAATCCTTACTACTACGACAGCAGCGGGTACTACTACCCAGGCGC
CTTTGATATCTGGGGCCAGGGCACAATGGTCACCGTGTCATCTGCATCTGTGGCCGC
TCCTAGCGTGTTCATCTTCCCACCTTCCGACGAACAGCTGAAGTCTGGCACAGCCAG
CGTCGTGTGCCTGCTGAACAACTTCTACCCCAGAGAAGCCAAGGTGCAGTGGAAGG
TGGACAACGCTCTGCAGTCCGGCAACAGCCAAGAGAGCGTGACAGAGCAGGACAG
CAAGGACTCCACCTACAGCCTGAGCAGCACCCTGACACTGAGCAAGGCCGACTACG
AGAAGCACAAAGTGTACGCCTGCGAAGTGACCCACCAGGGCCTTTCTAGCCCTGTG
ACCAAGAGCTTCAACCGGGGCGAGTGCGATAAGACACACACAGGCGGAAGCAGCG
GCAGCGGATCTGGATCTACCGGCACATCTAGCTCTGGCACCGGAACATCTGCCGGC
ACAACTGGCACAAGCGCCTCTACATCTGGAAGCGGTTCTGGCGGAGGCGGAGGATC
TGGTGGTGGTGGATCTGCTGGCGGAACAGCTACAGCTGGCGCTTCTAGCGGCAGCA
GCTATGTGCTGACACAGCCTCCATCCGTGTCTGTGGCACCTGGACAGACCGCCAGAA
TTACCTGTGGCGGCAACAACATCGGCAGCAAGAGCGTGCACTGGTATCAGCAGAAG
CCTGGACAGGCACCAGTGCTGGTGGTGTACGACGACAGCGATAGACCTAGCGGCAT
CCCCGAGAGATTCAGCGGCTCTAACAGCGGCAATACCGCCACACTGACCATCAGCA
GAGTGGAAGCTGGCGACGAGGCCGATTACTACTGCCAAGTGTGGGACAGCAGCAGC
CACCACTGGGTTTTCGGCGGAGGCACCAAACTGACAGTGCTGTCTAGCGCCAGCAC
AAAGGGCCCATCTGTGTTCCCTCTGGCTCCCAGCAGCAAGTCTACAAGCGGAGGAA
CAGCCGCTCTGGGCTGCCTCGTGAAGGATTACTTTCCCGAGCCTGTGACCGTGTCCT
GGAATAGCGGAGCACTGACAAGCGGCGTGCACACCTTTCCAGCTGTGCTGCAAAGC
TCCGGCCTGTACTCTCTGAGCAGCGTGGTCACAGTGCCTAGCTCTAGCCTGGGCACC
CAGACCTACATCTGCAATGTGAACCACAAGCCTAGCAACACCAAGGTCGACAAGAA
GGTGGAACCCAAGAGCTGCTGAGCCACGCGTAACACGTGCATGCGAGAGATCTGCG
GCCGCGAGCTCGGGGATCCAGACATGATAAGATACATTGATGAGTTTGGACAAACC
ACAACTAGAATGCAGTGAAAAAAATGCTTTATTTGTGAAATTTGTGATGCTATTGCT
TTATTTGTAACCATTATAAGCTGCAATAAACAAGTTAACAACAACAATTGCATTCAT
TTTATGTTTCAGGTTCAGGGGGAGGTGTGGGAGGTTTTTTAAAGCAAGTAAAACCTC
TACAAATGTGGTATGGCTGATTATGATCAATGCATCCTAGCCGGAGGAACCCCTAGT
GATGGAGTTGGCCACTCCCTCTCTGCGCGCTCGCTCGCTCACTGAGGCCGCCCGGGC
AAAGCCCGGGCGTCGGGCGACCTTTGGTCGCCCGGCCTCAGTGAGCGAGCGAGCGC
GCAGAGAGGGAGTGGCCAA

and an rAAV vector comprising a capsid protein of SEQ ID NO:48 and a nucleic acid comprising the following sequence (aflibercept+anti-Ang-2 LH construct) or a sequence at least 80%, at least 85%, at least 90%, at least 95%; at least 98% or at least 99% identical thereto:

(SEQ ID NO: 67)
TTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGAGA
GGGAGTGGCCAACTCCATCACTAGGGGTTCCTATCGATTGAATTCCAGGCGCGCCAT
CCTGCAGGTATTGACGTCAATAATGACGTATGTTCCCATAGTAACGCCAATAGGGAC
TTTCCATTGACGTCAATGGGTGGAGTATTTACGGTAAACTGCCCACTTGGCAGTACA
TCAAGTGTATCATATGCCAAGTACGCCCCCTATTGACGTCAATGACGGTAAATGGCC
CGCCTGGCATTATGCCCAGTACATGACCTTATGGGACTTTCCTACTTGGCAGTACAT
CTACGTATTAGTCATCGCTATTACCATGGTCGAGGTGAGCCCCACGTTCTGCTTCACT
CTCCCCATCTCCCCCCCCTCCCCACCCCCAATTTTGTATTTATTTATTTTTTAATTATT
TTGTGCAGCGATGGGGGGGGGGGGGGGGGGGGGGGGCGCGCCAGGCGGGGGGGGG
CGGGGCGAGGGGCGGGGGGGGGCGAGGCGGAGAGGTGCGGCGGCAGCCAATCAGA
GCGGCGCGCTCCGAAAGTTTCCTTTTATGGCGAGGCGGCGGCGGCGGCGGCCCTAT
AAAAAGCGAAGCGCGCGGCGGGCGGGAGTCGCTGCGCGCTGCCTTCGCCCCGTGCC
CCGCTCCGCCGCCGCCTCGCGCCGCCCGCCCCGGCTCTGACTGACCGCGTTACTCCC
ACAGGTGAGCGGGCGGGACGGCCCTTCTCCTCCGGGCTGTAATTAGCGCTTGGTTTA
ATGACGGCTTGTTTCTTTTCTGTGGCTGCGTGAAAGCCTTGAGGGGCTCCGGGAGGG
CCCTTTGTGGGGGGGGAGCGGCTCGGGGCTGTCCGCGGGGGGACGGCTGCCTTCGG
GGGGGACGGGGCAGGGGGGGGTTCGGCTTCTGGCGTGTGACCGGCGGCTCTAGAGC
CTCTGCTAACCATGTTCATGCCTTCTTCTTTTTCCTACAGCTCCTGGGCAACGTGCTG
GTTATTGTGCTGTCTCATCATTTTGGCAAAGAATTGTACAGGATATCTTGCTAGCACG
CCACCATGGTTTCTTACTGGGACACCGGCGTGCTGCTGTGTGCCCTGCTTTCTTGTCT
GCTGCTGACCGGCTCTAGCAGCGGCTCTGATACCGGCAGACCCTTCGTGGAAATGTA
CAGCGAGATCCCCGAGATCATCCACATGACCGAGGGCAGAGAGCTGGTCATCCCTT
GCAGAGTGACAAGCCCCAACATCACCGTGACTCTGAAGAAGTTCCCTCTGGACACA
CTGATCCCCGACGGCAAGAGAATCATCTGGGACAGCCGGAAGGGCTTCATCATCAG
CAACGCCACCTACAAAGAGATCGGCCTGCTGACCTGTGAAGCCACCGTGAATGGCC
ACCTGTACAAGACCAACTACCTGACACACAGACAGACCAACACCATCATCGACGTG
GTGCTGAGCCCTAGCCACGGCATTGAACTGTCTGTGGGCGAGAAGCTGGTGCTGAA
CTGTACCGCCAGAACCGAGCTGAACGTGGGCATCGACTTCAACTGGGAGTACCCCA
GCAGCAAGCACCAGCACAAGAAACTGGTCAACCGGGACCTGAAAACCCAGAGCGG
CAGCGAGATGAAGAAATTCCTGAGCACCCTGACCATCGACGGCGTGACCAGAAGTG
ACCAGGGCCTGTACACATGTGCCGCCAGCTCTGGCCTGATGACCAAGAAAAACAGC
ACCTTCGTGCGGGTGCACGAGAAGGACAAGACCCACACCTGTCCTCCATGTCCTGCT
CCAGAACTGCTCGGCGGACCTTCCGTGTTCCTGTTTCCTCCAAAGCCTAAGGACACC
CTGATGATCAGCAGAACCCCTGAAGTGACCTGCGTGGTGGTGGATGTGTCCCACGA
GGATCCCGAAGTGAAGTTCAATTGGTACGTGGACGGCGTGGAAGTGCACAACGCCA
AGACCAAGCCTAGAGAGGAACAGTACAATAGCACCTACAGAGTGGTGTCCGTGCTG
ACCGTGCTGCACCAGGATTGGCTGAACGGCAAAGAGTACAAGTGCAAGGTGTCCAA
CAAGGCCCTGCCTGCTCCTATCGAGAAAACCATCTCCAAGGCCAAGGGCCAGCCTA
GGGAACCCCAGGTTTACACACTGCCTCCAAGCAGGGACGAGCTGACAAAGAACCAG
GTGTCCCTGACCTGCCTGGTCAAGGGCTTCTACCCTTCCGATATCGCCGTGGAATGG
GAGAGCAATGGCCAGCCTGAGAACAACTACAAGACAACCCCTCCTGTGCTGGACAG
CGACGGCTCATTCTTCCTGTACAGCAAGCTGACAGTGGACAAGAGCAGATGGCAGC
AGGGCAACGTGTTCAGCTGCTCCGTGATGCACGAGGCCCTGCACAACCACTACACC
CAGAAGTCCCTGAGCCTGTCTCCTGGCAAACGGAAGAGAAGAGGCAGCGGCGAAG
GCAGAGGATCCCTGCTTACATGTGGCGACGTGGAAGAGAACCCCGGACCTATGGTT
CTGCAGACCCAGGTGTTCATCAGCCTGCTGCTGTGGATCTCTGGCGCCTACGGCAGC
TATGTGCTGACACAGCCTCCATCCGTGTCTGTGGCTCCAGGACAGACCGCCAGAATT
ACCTGCGGCGGCAACAACATCGGCAGCAAGAGCGTGCACTGGTATCAGCAGAAGCC
TGGACAGGCTCCAGTGCTGGTGGTGTACGACGACAGCGATAGACCTAGCGGCATCC
CCGAGAGATTCAGCGGCAGCAATTCCGGCAATACCGCCACACTGACCATCAGCAGA
GTGGAAGCTGGCGACGAGGCCGACTACTACTGCCAAGTGTGGGATAGCAGCAGCGA
CCACTGGGTTTTCGGCGGAGGCACCAAACTGACAGTGCTGAGCAGCGCCTCTACAA
AGGGCCCTAGTGTGTTCCCTCTGGCTCCCAGCAGCAAGTCTACATCTGGCGGAACAG
CCGCTCTGGGCTGCCTCGTGAAGGATTACTTTCCCGAGCCTGTGACCGTGTCCTGGA
ATAGCGGAGCACTGACAAGCGGCGTGCACACCTTTCCAGCTGTGCTGCAAAGCAGC
GGCCTGTACTCTCTGTCCAGCGTGGTCACAGTGCCAAGCTCTAGCCTGGGCACCCAG
ACCTACATCTGCAATGTGAACCACAAGCCTAGCAACACCAAGGTCGACAAGAAGGT
GGAACCCAAGAGCTGTGGCGGCAGCTCTGGTTCTGGATCTGGCAGCACAGGCACAT
CTAGCTCTGGCACCGGAACAAGCGCTGGCACAACTGGCACATCTGCCAGCACAAGC
GGATCTGGAAGTGGCGGAGGCGGAGGATCTGGTGGCGGTGGATCTGCAGGCGGAAC
TGCTACAGCTGGCGCTTCTAGTGGAAGCCAGGTGCAGCTGGTTCAGTCTGGCGCCGA
AGTGAAAAAGCCTGGCGCCTCTGTGAAGGTGTCCTGCAAGGCCAGCGGCTACACCT
TTACCGGCTACTACATGCACTGGGTCCGACAGGCACCAGGACAGGGACTTGAATGG
ATGGGCTGGATCAACCCCAATAGCGGCGGCACCAATTACGCCCAGAAATTCCAGGG
CAGAGTGACCATGACCAGAGACACCAGCATCAGCACCGCCTACATGGAACTGAGCC
GGCTGAGATCCGATGACACCGCCGTGTACTACTGCGCCAGATCTCCCAATCCTTACT
ACTACGACAGCAGCGGGTACTACTACCCAGGCGCCTTTGATATCTGGGGCCAGGGC
ACCATGGTCACCGTGTCATCTGCATCTGTGGCCGCTCCTAGCGTGTTCATCTTCCCAC
CTTCCGACGAACAGCTGAAGTCCGGCACAGCCTCTGTCGTGTGCCTGCTGAACAACT
TCTACCCCAGAGAAGCCAAAGTGCAGTGGAAGGTGGACAACGCCCTGCAGAGCGGC
AATAGCCAAGAGAGCGTGACCGAGCAGGACAGCAAGGACTCTACCTACAGCCTGAG
CAGCACCCTGACACTGAGCAAGGCCGATTACGAGAAGCACAAGGTGTACGCCTGCG
AAGTGACACACCAGGGCCTGTCTAGCCCTGTGACCAAGAGCTTCAATCGGGGCGAG
TGCGACAAGACCCACACCTAAGCCACGCGTAACACGTGCATGCGAGAGATCTGCGG
CCGCGAGCTCGGGGATCCAGACATGATAAGATACATTGATGAGTTTGGACAAACCA
CAACTAGAATGCAGTGAAAAAAATGCTTTATTTGTGAAATTTGTGATGCTATTGCTT
TATTTGTAACCATTATAAGCTGCAATAAACAAGTTAACAACAACAATTGCATTCATT
TTATGTTTCAGGTTCAGGGGGAGGTGTGGGAGGTTTTTTAAAGCAAGTAAAACCTCT
ACAAATGTGGTATGGCTGATTATGATCAATGCATCCTAGCCGGAGGAACCCCTAGTG
ATGGAGTTGGCCACTCCCTCTCTGCGCGCTCGCTCGCTCACTGAGGCCGCCCGGGCA
AAGCCCGGGCGTCGGGCGACCTTTGGTCGCCCGGCCTCAGTGAGCGAGCGAGCGCG
CAGAGAGGGAGTGGCCAA

and their use in treating wet (neovascular, exudative) age-related macular degeneration; diabetic macular edema; macular edema following retinal vein occlusion; diabetic retinopathy; or myopic choroidal neovascularization, preferably wherein the vector is intraocularly administered to a human subject, preferably wherein intraocular administration comprises intravitreal injection (e.g., a single intravitreal injection), subretinal injection or suprachoroidal injection.

In some aspects, the nucleic acid comprises a nucleotide sequence encoding aflibercept and a nucleotide sequence encoding an interfering RNA that reduces expression of a pro-angiogenic protein. In preferred embodiments, the nucleotide sequence encoding an interfering RNA encodes a natural or artificial miRNA comprising a sense strand and antisense strand that reduces expression of a pro-angiogenic protein.

In related aspects, the interfering RNA reduces expression of human ang-1 and/or human ang-2. In particularly preferred embodiments, an rAAV vector is provided comprising a capsid protein of SEQ ID NO:48 and a nucleic acid comprising the following sequence (aflibercept+human Ang-2 interfering RNA (SEQ ID NO:13) construct)) or a sequence at least 80%, at least 90%, at least 95%, at least 98% or at least 99% identical thereto:

(SEQ ID NO: 68)
TTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGAGA
GGGAGTGGCCAACTCCATCACTAGGGGTTCCTATCGATTGAATTCCCCGGGGATCCA
CTAGTTATTAATAGTAATCAATTACGGGGTCATTAGTTCATAGCCCATATATGGAGT
TCCGCGTTACATAACTTACGGTAAATGGCCCGCCTGGCTGACCGCCCAACGACCCCC
GCCCATTGACGTCAATAATGACGTATGTTCCCATAGTAACGCCAATAGGGACTTTCC
ATTGACGTCAATGGGTGGAGTATTTACGGTAAACTGCCCACTTGGCAGTACATCAAG
TGTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACG
TATTAGTCATCGCTATTACCATGGTCGAGGTGAGCCCCACGTTCTGCTTCACTCTCCC
CATCTCCCCCCCCTCCCCACCCCCAATTTTGTATTTATTTATTTTTTAATTATTTTGTG
CAGCGATGGGGGGGGGGGGGGGGGGGGGGGGCGCGCCAGGCGGGGGGGGGGGGG
GCGAGGGGCGGGGCGGGGCGAGGCGGAGAGGTGCGGCGGCAGCCAATCAGAGCGG
CGCGCTCCGAAAGTTTCCTTTTATGGCGAGGCGGCGGCGGCGGCGGCCCTATAAAA
AGCAAGCGCGCGGCGGGCGGGGAGTCGCTGCGACGCTGCCTTCGCCCCGTGCCCC
GCTCCGCCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCGCTTGGTTTAAT
GACGGCTTGTTTCTTTTCTGTGGCTGCGTGAAAGCCTTGAGGGGCTCCGGGAGGGCC
CTTTGTGCGGGGGGAGCGGCTCGGGGGGTGCGTGCGTGTGTGTGTGCGTGGGGAGC
GCCGCGTGCGGCTCCGCGCTGCCCGGCGGCTGTGAGCGCTGCGGGCGCGGCGCGGG
GCTTTGTGCGCTCCGCAGTGTGCGCGAGGGGAGCGCGGCCGGGGGCGGTGCCCCGC
GGTGCGGGGGGGGCTGCGAGGGGAACAAAGGCTGCGTGCGGGGTGTGTGCGTGGG
GGGGTGAGCAGGGGGTGTGGGCGCGTCGGTCGGGCTGCAACCCCCCCTGCACCCCC
CTCCCCGAGTTGCTGAGCACGGCCCGGCTTCGGGTGCGGGGCTCCGTACGGGGCGT
GGCGCGGGGCTCGCCGTGCCGGGGGGGGGGGGCGGCAGGTGGGGGTGCCGGGCG
GGGGGGGGCCGCCTGGGGCCGGGGAGGGCTCGGGGGAGGGGGGGGGGGGCCCCCG
GAGCGCCGGCGCGACTTCTTAACCCAACAGAAGGCTCGAGAAGGTATATTGCTGTT
GACAGTGAGCGGCTTACTCATTGTATGAACATTTAGTGAAGCCACAGATGTAAATGT
TCATACAATGAGTAAGCTGCCTACTGCCTCGGACTTCAAGGGGCTAGAATTCGCGGC
TGTCGAGGCGCGGCGAGCCGCAGCCATTGCCTTTTATGGTAATCGTGCGAGAGGGC
GCAGGGACTTCCTTTGTCCCAAATCTGTGCGGAGCCGAAATCTGGGAGGCGCCGCC
GCACCCCCTCTAGCGGGCGCGGGGCGAAGCGGTGCGGCGCCGGCAGGAAGGAAAT
GGGGGGGGAGGGCCTTCGTGCGTCGCCGCGCCGCCGTCCCCTTCTCCCTCTCCAGCC
TCGGGGCTGTCCGCGGGGGGACGGCTGCCTTCGGGGGGGACGGGGCAGGGGGGGGT
TCGGCTTCTGGCGTGTGACCGGCGGCTCTAGAGCCTCTGCTAACCATGTTCATGCCTT
CTTCTTTTTCCTACAGTCTAGAGTCGACCTGCAGGTGGATATCTTGGCTAGCACGCCA
CCATGGTTTCTTACTGGGACACCGGCGTGCTGCTGTGTGCCCTGCTTTCTTGTCTGCT
GCTGACCGGCTCTAGCAGCGGCTCTGATACCGGCAGACCCTTCGTGGAAATGTACA
GCGAGATCCCCGAGATCATCCACATGACCGAGGGCAGAGAGCTGGTCATCCCTTGC
AGAGTGACAAGCCCCAACATCACCGTGACTCTGAAGAAGTTCCCTCTGGACACACT
GATCCCCGACGGCAAGAGAATCATCTGGGACAGCCGGAAGGGCTTCATCATCAGCA
ACGCCACCTACAAAGAGATCGGCCTGCTGACCTGTGAAGCCACCGTGAATGGCCAC
CTGTACAAGACCAACTACCTGACACACAGACAGACCAACACCATCATCGACGTGGT
GCTGAGCCCTAGCCACGGCATTGAACTGTCTGTGGGCGAGAAGCTGGTGCTGAACT
GTACCGCCAGAACCGAGCTGAACGTGGGCATCGACTTCAACTGGGAGTACCCCAGC
AGCAAGCACCAGCACAAGAAACTGGTCAACCGGGACCTGAAAACCCAGAGCGGCA
GCGAGATGAAGAAATTCCTGAGCACCCTGACCATCGACGGCGTGACCAGAAGTGAC
CAGGGCCTGTACACATGTGCCGCCAGCTCTGGCCTGATGACCAAGAAAAACAGCAC
CTTCGTGCGGGTGCACGAGAAGGACAAGACCCACACCTGTCCTCCATGTCCTGCTCC
AGAACTGCTCGGCGGACCTTCCGTGTTCCTGTTTCCTCCAAAGCCTAAGGACACCCT
GATGATCAGCAGAACCCCTGAAGTGACCTGCGTGGTGGTGGATGTGTCCCACGAGG
ATCCCGAAGTGAAGTTCAATTGGTACGTGGACGGCGTGGAAGTGCACAACGCCAAG
ACCAAGCCTAGAGAGGAACAGTACAATAGCACCTACAGAGTGGTGTCCGTGCTGAC
CGTGCTGCACCAGGATTGGCTGAACGGCAAAGAGTACAAGTGCAAGGTGTCCAACA
AGGCCCTGCCTGCTCCTATCGAGAAAACCATCTCCAAGGCCAAGGGCCAGCCTAGG
GAACCCCAGGTTTACACACTGCCTCCAAGCAGGGACGAGCTGACAAAGAACCAGGT
GTCCCTGACCTGCCTGGTCAAGGGCTTCTACCCTTCCGATATCGCCGTGGAATGGGA
GAGCAATGGCCAGCCTGAGAACAACTACAAGACAACCCCTCCTGTGCTGGACAGCG
ACGGCTCATTCTTCCTGTACAGCAAGCTGACAGTGGACAAGAGCAGATGGCAGCAG
GGCAACGTGTTCAGCTGCTCCGTGATGCACGAGGCCCTGCACAACCACTACACCCA
GAAGTCCCTGAGCCTGTCTCCTGGCAAATGAGCCACGCGTAACACGTGGGGGATCC
AGACATGATAAGATACATTGATGAGTTTGGACAAACCACAACTAGAATGCAGTGAA
AAAAATGCTTTATTTGTGAAATTTGTGATGCTATTGCTTTATTTGTAACCATTATAAG
CTGCAATAAACAAGTTAACAACAACAATTGCATTCATTTTATGTTTCAGGTTCAGGG
GGAGGTGTGGGAGGTTTTTTAAAGCAAGTAAAACCTCTACAAATGTGGTATGGCTG
ATTATGATCAATGCATGGCCGGCCGGAGGAACCCCTAGTGATGGAGTTGGCCACTCC
CTCTCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCA
A

and its use in treating wet (neovascular, exudative) age-related macular degeneration; diabetic macular edema; macular edema following retinal vein occlusion; diabetic retinopathy; or myopic choroidal neovascularization, preferably wherein the vector is intraocularly administered to a human subject, preferably wherein intraocular administration comprises intravitreal injection (e.g., a single intravitreal injection), subretinal injection or suprachoroidal injection.

In related aspects, the interfering RNA reduces expression of human VEGF-C. In particularly preferred embodiments, an rAAV vector is provided comprising a capsid protein of SEQ ID NO:48 and a nucleic acid comprising the following sequence (aflibercept+human VEGF-C interfering RNA (SEQ ID NOs:19/20) construct) or a sequence at least 80%, at least 90%, at least 95%, at least 98% or at least 99% identical thereto:

(SEQ ID NO: 69)
TTGGCCACTCCCTCTCTGCGCGCTCGCTCGCTCACTGAGGCCGGGCGACCAAAGGTC
GCCCGACGCCCGGGCTTTGCCCGGGGGGCCTCAGTGAGCGAGCGAGCGCGCAGAGA
GGGAGTGGCCAACTCCATCACTAGGGGTTCCTATCGATTGAATTCCCCGGGGATCCA
CTAGTTATTAATAGTAATCAATTACGGGGTCATTAGTTCATAGCCCATATATGGAGT
TCCGCGTTACATAACTTACGGTAAATGGCCCGCCTGGCTGACCGCCCAACGACCCCC
GCCCATTGACGTCAATAATGACGTATGTTCCCATAGTAACGCCAATAGGGACTTTCC
ATTGACGTCAATGGGTGGAGTATTTACGGTAAACTGCCCACTTGGCAGTACATCAAG
TGTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACG
TATTAGTCATCGCTATTACCATGGTCGAGGTGAGCCCCACGTTCTGCTTCACTCTCCC
CATCTCCCCCCCCTCCCCACCCCCAATTTTGTATTTATTTATTTTTTAATTATTTTGTG
CAGCGATGGGGGGGGGGGGGGGGGGGGGGGGCGCGCCAGGCGGGGGGGGGCGGG
GCGAGGGGCGGGGGGGGCGAGGCGGAGAGGTGCGGCGGCAGCCAATCAGAGCGG
CGCGCTCCGAAAGTTTCCTTTTATGGCGAGGCGGCGGCGGCGGCGGCCCTATAAAA
AGCGAAGCGCGCGGCGGGGGGGGAGTCGCTGCGACGCTGCCTTCGCCCCGTGCCCC
GCTCCGCCGCCGCCTCGCGCCGCCCGCCCCGGCTCTGACTGACCGCGTTACTGCCAC
AGGTGAGCGGGGGGGACGGCCCTTCTCCTCCGGGCTGTAATTAGCGCTTGGTTTAAT
GACGGCTTGTTTCTTTTCTGTGGCTGCGTGAAAGCCTTGAGGGGCTCCGGGAGGGCC
CTTTGTGCGGGGGGAGCGGCTCGGGGGGTGCGTGCGTGTGTGTGTGCGTGGGGAGC
GCCGCGTGCGGCTCCGCGCTGCCCGGCGGCTGTGAGCGCTGCGGGCGCGGCGCGGG
GCTTTGTGCGCTCCGCAGTGTGCGCGAGGGGAGCGCGGCCGGGGGGGGTGCCCCGC
GGTGCGGGGGGGGCTGCGAGGGGAACAAAGGCTGCGTGCGGGGTGTGTGCGTGGG
GGGGTGAGCAGGGGGTGTGGGCGCGTCGGTCGGGCTGCAACCCCCCCTGCACCCCC
CTCCCCGAGTTGCTGAGCACGGCCCGGCTTCGGGTGCGGGGCTCCGTACGGGGCGT
GGCGCGGGGCTCGCCGTGCCGGGCGGGGGGTGGCGGCAGGTGGGGGTGCCGGGCG
GGGCGGGGCCGCCTCGGGCCGGGGAGGGCTCGGGGGAGGGGCGCGGCGGCCCCCG
GAGCGCCGGCGCGACTTCTTAACCCAACAGAAGGCTCGAGAAGGTATATTGCTGTT
GACAGTGAGCGGCTACCTCAGCAAGACGTTATTTAGTGAAGCCACAGATGTAAATA
ACGTCTTGCTGAGGTAGCTGCCTACTGCCTCGGACTTCAAGGGGCTAGAATTCGCGG
CTGTCGAGGCGCGGCGAGCCGCAGCCATTGCCTTTTATGGTAATCGTGCGAGAGGGC
GCAGGGACTTCCTTTGTCCCAAATCTGTGCGGAGCCGAAATCTGGGAGGCGCCGCC
GCACCCCCTCTAGCGGGCGCGGGGCGAAGCGGTGCGGCGCCGGCAGGAAGGAAAT
GGGCGGGGAGGGCCTTCGTGCGTCGCCGCGCCGCCGTCCCCTTCTCCCTCTCCAGCC
TCGGGGCTGTCCGCGGGGGGACGGCTGCCTTCGGGGGGGACGGGGCAGGGGGGGGT
TCGGCTTCTGGCGTGTGACCGGCGGCTCTAGAGCCTCTGCTAACCATGTTCATGCCTT
CTTCTTTTTCCTACAGTCTAGAGTCGACCTGCAGGTGGATATCTTGGCTAGCACGCCA
CCATGGTTTCTTACTGGGACACCGGCGTGCTGCTGTGTGCCCTGCTTTCTTGTCTGCT
GCTGACCGGCTCTAGCAGCGGCTCTGATACCGGCAGACCCTTCGTGGAAATGTACA
GCGAGATCCCCGAGATCATCCACATGACCGAGGGCAGAGAGCTGGTCATCCCTTGC
AGAGTGACAAGCCCCAACATCACCGTGACTCTGAAGAAGTTCCCTCTGGACACACT
GATCCCCGACGGCAAGAGAATCATCTGGGACAGCCGGAAGGGCTTCATCATCAGCA
ACGCCACCTACAAAGAGATCGGCCTGCTGACCTGTGAAGCCACCGTGAATGGCCAC
CTGTACAAGACCAACTACCTGACACACAGACAGACCAACACCATCATCGACGTGGT
GCTGAGCCCTAGCCACGGCATTGAACTGTCTGTGGGCGAGAAGCTGGTGCTGAACT
GTACCGCCAGAACCGAGCTGAACGTGGGCATCGACTTCAACTGGGAGTACCCCAGC
AGCAAGCACCAGCACAAGAAACTGGTCAACCGGGACCTGAAAACCCAGAGCGGCA
GCGAGATGAAGAAATTCCTGAGCACCCTGACCATCGACGGCGTGACCAGAAGTGAC
CAGGGCCTGTACACATGTGCCGCCAGCTCTGGCCTGATGACCAAGAAAAACAGCAC
CTTCGTGCGGGTGCACGAGAAGGACAAGACCCACACCTGTCCTCCATGTCCTGCTGC
AGAACTGCTCGGCGGACCTTCCGTGTTCCTGTTTCCTCCAAAGCCTAAGGACACCCT
GATGATCAGCAGAACCCCTGAAGTGACCTGCGTGGTGGTGGATGTGTCCCACGAGG
ATCCCGAAGTGAAGTTCAATTGGTACGTGGACGGCGTGGAAGTGCACAACGCCAAG
ACCAAGCCTAGAGAGGAACAGTACAATAGCACCTACAGAGTGGTGTCCGTGCTGAC
CGTGCTGCACCAGGATTGGCTGAACGGCAAAGAGTACAAGTGCAAGGTGTCCAACA
AGGCCCTGCCTGCTCCTATCGAGAAAACCATCTCCAAGGCCAAGGGCCAGCCTAGG
GAACCCCAGGTTTACACACTGCCTCCAAGCAGGGACGAGCTGACAAAGAACCAGGT
GTCCCTGACCTGCCTGGTCAAGGGCTTCTACCCTTCCGATATCGCCGTGGAATGGGA
GAGCAATGGCCAGCCTGAGAACAACTACAAGACAACCCCTCCTGTGCTGGACAGCG
ACGGCTCATTCTTCCTGTACAGCAAGCTGACAGTGGACAAGAGCAGATGGCAGCAG
GGCAACGTGTTCAGCTGCTCCGTGATGCACGAGGCCCTGCACAACCACTACACCCA
GAAGTCCCTGAGCCTGTCTCCTGGCAAATGAGCCACGCGTAACACGTGGGGGATCC
AGACATGATAAGATACATTGATGAGTTTGGACAAACCACAACTAGAATGCAGTGAA
AAAAATGCTTTATTTGTGAAATTTGTGATGCTATTGCTTTATTTGTAACCATTATAAG
CTGCAATAAACAAGTTAACAACAACAATTGCATTCATTTTATGTTTCAGGTTCAGGG
GGAGGTGTGGGAGGTTTTTTAAAGCAAGTAAAACCTCTACAAATGTGGTATGGCTG
ATTATGATCAATGCATGGCCGGCCGGAGGAACCCCTAGTGATGGAGTTGGCCACTCC
CTCTCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCA
A

and its use in treating wet (neovascular, exudative) age-related macular degeneration; diabetic macular edema; macular edema following retinal vein occlusion; diabetic retinopathy; or myopic choroidal neovascularization, preferably wherein the vector is intraocularly administered to a human subject, preferably wherein intraocular administration comprises intravitreal injection (e.g., a single intravitreal injection), subretinal injection or suprachoroidal injection.

In related aspects, the interfering RNA reduces expression of human VEGFR-3. In particularly preferred embodiments, an rAAV vector is provided comprising a capsid protein of SEQ ID NO:48 and a nucleic acid comprising the following sequence (aflibercept+human VEGFR-3 interfering RNA (SEQ ID NO:37) construct) or a sequence at least 80%, at least 90%, at least 95%, at least 98% or at least 99% identical thereto:

(SEQ ID NO: 70)
TTGGCCACTCCCTCTCTGCGCGCTCGCTCGCTCACTGAGGCCGGGCGACCAAAGGTC
CCCCGACGCCCGGGCTTTGCCCGGGCGGCCTCAGTGAGCGAGCGAGCGCGCAGAGA
GGGAGTGGCCAACTCCATCACTAGGGGTTCCTATCGATTGAATTCCCCGGGGATCCA
CTAGTTATTAATAGTAATCAATTACGGGGTCATTAGTTCATAGCCCATATATGGAGT
TCCGCGTTACATAACTTACGGTAAATGGCCCGCCTGGCTGACCGCCCAACGACCCCC
GCCCATTGACGTCAATAATGACGTATGTTCCCATAGTAACGCCAATAGGGACTTTCC
ATTGACGTCAATGGGTGGAGTATTTACGGTAAACTGCCCACTTGGCAGTACATCAAG
TGTATCATATGCCAAGTACGCCCCCTATTGACGTCAATGACGGTAAATGGCCCGGCT
GGCATTATGCCCAGTACATGACCTTATGGGACTTTCCTACTTGGCAGTACATCTACG
TATTAGTCATCGCTATTACCATGGTCGAGGTGAGCCCCACGTTCTGCTTCACTCTCCC
CATCTCCCCCCCCTCCCCACCCCCAATTTTGTATTTATTTATTTTTTAATTATTTTGTG
CAGCGATGGGGGGGGGGGGGGGGGGGGGGGGCGCGCCAGGCGGGGGGGGGGGGG
GCGAGGGGGGGGGGGGGGCGAGGCGGAGAGGTGCGGCGGCAGCCAATCAGAGCGG
CGCGCTCCGAAAGTTTCCTTTTATGGCGAGGCGGCGGCGGCGGCGGCCCTATAAAA
AGCGAAGCGCGCGGCGGGGGGGGAGTCGCTGCGACGCTGCCTTCGCCCCGTGCCCC
GCTCCGCCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCGCTTGGTTTAAT
GACGGCTTGTTTCTTTTCTGTGGCTGCGTGAAAGCCTTGAGGGGCTCCGGGAGGGCC
CTTTGTGCGGGGGGAGCGGCTCGGGGGGTGCGTGCGTGTGTGTGTGCGTGGGGAGC
GCCGCGTGCGGCTCCGCGCTGCCCGGCGGCTGTGAGCGCTGCGGGCGCGGCGCGGG
GCTTTGTGCGCTCCGCAGTGTGCGCGAGGGGAGCGCGGCCGGGGGGGGTGCCCCGC
GGTGCGGGGGGGGCTGCGAGGGGAACAAAGGCTGCGTGCGGGGTGTGTGCGTGGG
GGGGTGAGCAGGGGGTGTGGGCGCGTCGGTCGGGCTGCAACCCCCCCTGCACCCCC
CTCCCCGAGTTGCTGAGCACGGCCCGGCTTCGGGTGCGGGGCTCCGTACGGGGCGT
GGCGCGGGGCTCGCCGTGCCGGGGGGGGGGGGCGGCAGGTGGGGGTGCCGGGCG
GGGGGGGGCCGCCTCGGGCCGGGGAGGGCTCGGGGGAGGGGCGCGGGGGCCCCCG
GAGCGCCGGCGCGACTTCTTAACCCAACAGAAGGCTCGAGAAGGTATATTGCTGTT
GACAGTGAGCGCACCGTGTGGGCTGAGTTTAACTAGTGAAGCCACAGATGTAGTTA
AACTCAGCCCACACGGTGTGCCTACTGCCTCGGACTTCAAGGGGCTAGAATTCGCGG
CTGTCGAGGCGCGGCGAGCCGCAGCCATTGCCTTTTATGGTAATCGTGCGAGAGGGC
GCAGGGACTTCCTTTGTGCCAAATCTGTGCGGAGCCGAAATCTGGGAGGCGCCGCC
GCACCCCCTCTAGCGGGCGCGGGGCGAAGCGGTGCGGCGCCGGCAGGAAGGAAAT
GGGCGGGGAGGGCCTTCGTGCGTCGCCGCGCCGCCGTCCCCTTCTCCCTCTCCAGCC
TCGGGGCTGTCCGCGGGGGGACGGCTGCCTTCGGGGGGGACGGGGCAGGGGGGGGT
TCGGCTTCTGGCGTGTGACCGGCGGCTCTAGAGCCTCTGCTAACCATGTTCATGCCTT
CTTCTTTTTCCTACAGTCTAGAGTCGACCTGCAGGTGGATATCTTGGCTAGCACGCCA
CCATGGTTTCTTACTGGGACACCGGCGTGCTGCTGTGTGCCCTGCTTTCTTGTCTGCT
GCTGACCGGCTCTAGCAGCGGCTCTGATACCGGCAGACCCTTCGTGGAAATGTACA
GCGAGATCCCCGAGATCATCCACATGACCGAGGGCAGAGAGCTGGTCATCCCTTGC
AGAGTGACAAGCCCCAACATCACCGTGACTCTGAAGAAGTTCCCTCTGGACACACT
GATCCCCGACGGCAAGAGAATCATCTGGGACAGCCGGAAGGGCTTCATCATCAGCA
ACGCCACCTACAAAGAGATCGGCCTGCTGACCTGTGAAGCCACCGTGAATGGCCAC
CTGTACAAGACCAACTACCTGACACACAGACAGACCAACACCATCATCGACGTGGT
GCTGAGCCCTAGCCACGGCATTGAACTGTCTGTGGGCGAGAAGCTGGTGCTGAACT
GTACCGCCAGAACCGAGCTGAACGTGGGCATCGACTTCAACTGGGAGTACCCCAGC
AGCAAGCACCAGCACAAGAAACTGGTCAACCGGGACCTGAAAACCCAGAGCGGCA
GCGAGATGAAGAAATTCCTGAGCACCCTGACCATCGACGGCGTGACCAGAAGTGAC
CAGGGCCTGTACACATGTGCCGCCAGCTCTGGCCTGATGACCAAGAAAAACAGCAC
CTTCGTGCGGGTGCACGAGAAGGACAAGACCCACACCTGTCCTCCATGTCCTGCTGC
AGAACTGCTCGGCGGACCTTCCGTGTTCCTGTTTCCTCCAAAGCCTAAGGACACCCT
GATGATCAGCAGAACCCCTGAAGTGACCTGCGTGGTGGTGGATGTGTCCCACGAGG
ATCCCGAAGTGAAGTTCAATTGGTACGTGGACGGCGTGGAAGTGCACAACGCCAAG
ACCAAGCCTAGAGAGGAACAGTACAATAGCACCTACAGAGTGGTGTCCGTGCTGAC
CGTGCTGCACCAGGATTGGCTGAACGGCAAAGAGTACAAGTGCAAGGTGTCCAACA
AGGCCCTGCCTGCTCCTATCGAGAAAACCATCTCCAAGGCCAAGGGCCAGCCTAGG
GAACCCCAGGTTTACACACTGCCTGCAAGCAGGGACGAGCTGACAAAGAACCAGGT
GTCCCTGACCTGCCTGGTCAAGGGCTTCTACCCTTCCGATATCGCCGTGGAATGGGA
GAGCAATGGCCAGCCTGAGAACAACTACAAGACAACCCCTCCTGTGCTGGACAGCG
ACGGCTCATTCTTCCTGTACAGCAAGCTGACAGTGGACAAGAGCAGATGGCAGCAG
GGCAACGTGTTCAGCTGCTCCGTGATGCACGAGGCCCTGCACAACCACTACACCCA
GAAGTCCCTGAGCCTGTCTCCTGGCAAATGAGCCACGCGTAACACGTGGGGGATCC
AGACATGATAAGATACATTGATGAGTTTGGACAAACCACAACTAGAATGCAGTGAA
AAAAATGCTTTATTTGTGAAATTTGTGATGCTATTGCTTTATTTGTAACCATTATAAG
CTGCAATAAACAAGTTAACAACAACAATTGCATTCATTTTATGTTTCAGGTTCAGGG
GGAGGTGTGGGAGGTTTTTTAAAGCAAGTAAAACCTCTACAAATGTGGTATGGCTG
ATTATGATCAATGCATGGCCGGCCGGAGGAACCCCTAGTGATGGAGTTGGCCACTCC
CTCTCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCA
A

and its use in treating wet (neovascular, exudative) age-related macular degeneration; diabetic macular edema; macular edema following retinal vein occlusion; diabetic retinopathy; or myopic choroidal neovascularization, preferably wherein the vector is intraocularly administered to a human subject, preferably wherein intraocular administration comprises intravitreal injection (e.g., a single intravitreal injection), subretinal injection or suprachoroidal injection is administered to a subject, preferably by intravitreal injection (e.g., a single intravitreal injection), to treat wet (neovascular, exudative) age-related macular degeneration; diabetic macular edema; macular edema following retinal vein occlusion; diabetic retinopathy; or myopic choroidal neovascularization.

Also provided herein are pharmaceutical compositions comprising: a) a nucleic acid as herein described, preferably encapsidated within an rAAV (preferably an rAAV comprising a capsid protein of SEQ ID NO:48) and; and b) a pharmaceutically acceptable carrier, diluent, excipient, or buffer. In some preferred embodiments, the nucleic acid comprises a nucleotide sequence selected from SEQ ID Nos:64-70. In some embodiments, the pharmaceutically acceptable carrier, diluent, excipient, or buffer is suitable for use in a human or non-human patient. Such excipients, carriers, diluents, and buffers include any pharmaceutical agent that can be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, surfactants, pH buffering substances, and the like, may be present in such vehicles. A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

In some preferred embodiments, the pharmaceutical composition comprises Dulbecco's Phosphate Buffered Saline (DPBS) and a non-ionic surfactant (e.g., Pluronic F68, preferably at about 0.005%).

In some embodiments, the pharmaceutical composition comprises 1×10⁸ to 1×10¹⁵ vector particles or vector genomes, 1×10¹⁰ to 1×10¹³ vector particles or vector genomes, or about 1×10¹⁰, about 2×10¹⁰), 3×10¹⁰, about 4×10¹⁰, about 5×10¹⁰, about 6×10¹⁰, about 7×10¹⁰, about 8×10¹⁰, about 9×10¹⁰, about 1×10¹¹, about 2×10¹¹, about 3×10¹¹, about 4×10¹¹, about 5×10¹¹, about 6×10¹¹, about 7×10¹¹, about 8×10¹¹, about 9×10¹¹, about 1×10¹², about 2×10¹², about 3×10¹², about 4×10¹², about 5×10¹², about 6×10¹², about 7×10¹², about 8×10¹², about 9×10¹² or about 1×10¹³ vector particles or vector genomes. In some aspects, the pharmaceutical composition comprises about 1×10¹¹ to about 1×10¹² vector particles or vector genomes.

In some preferred embodiments, the pharmaceutical composition is administered intraocularly to a human with a VEGF-related ocular disorder, preferably wherein the pharmaceutical composition is administered via intravitreal, subretinal and/or suprachoroidal injection, more preferably via a single intravitreal injection.

### EXAMPLES

The following examples illustrate preferred embodiments of the present invention and are not intended to limit the scope of the invention in any way. While this invention has been described in relation to its preferred embodiments, various modifications thereof will be apparent to one skilled in the art from reading this application.

### Example 1

The following examples describe multi-mechanistic approaches to anti-angiogenic gene therapy with recombinant adeno-associated virus (rAAV) comprising a genetically modified capsid protein that confers improved transduction of a panoply of retinal cells. The rAAV constructs described below provide sustained delivery of anti-angiogenic agents from a single intravitreal dose, limiting the burden of repeated injections, maintaining consistent levels of therapeutic gene products in pertinent retinal cells and improving therapeutic response compared to delivery of single anti-angiogenic agents. Each of the representative constructs described below comprises a nucleic acid encoding aflibercept (targeting VEGF-A, VEGF-B and PIGF) and at least one other anti-angiogenic agent to enhance efficacy beyond delivery of aflibercept alone.

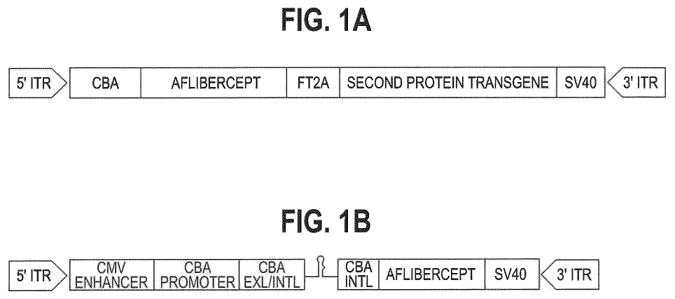

Representative construct designs (AAV vector backbones) are illustrated at FIGS. 1A-B. In the first approach (FIG. 1A), nucleotide sequence encoding aflibercept and a second anti-angiogenic polypeptide, separated by an FT2A (ribosomal-skipping peptide) sequence, are controlled by a ubiquitous CBA promoter (it is a bicistronic construct). Second anti-angiogenic polypeptides exemplified herein include PEDF, VEGFR-3-Fc fusion protein and Anti-Ang-2 scFab fragments, selected on the basis of their function in reducing angiogenesis and/or vascular permeability. In each case, the encoding gene was codon-optimized for human expression. In the second approach (FIG. 1B), nucleotide sequence encoding aflibercept is driven by a CAG promoter with nucleotide sequence encoding an interfering RNA that targets a pro-angiogenic protein. In each case, the RNAi sequence was embedded in the well-characterized miR-E backbone (as described e.g., in US Patent Application Publication No. 2015-0018539, the contents of which are incorporated herein by reference and Fellmann et al., Cell Reports, 5(6):1704-1713 (2013)) and placed within the hybrid chicken b-actin/rabbit b-globin intron (See FIG. 1B). The CBA promoter is a well-characterized ubiquitous promoter capable of driving sustained, high levels of expression. The CBA promoter is a hybrid of the human cytomegalovirus (CMV) upstream enhancer with the chicken β-actin (CBA) promoter and also contains chicken b-actin exon 1, a hybrid chicken b-actin and rabbit b-globin intron and a rabbit b-globin exon 3 fragment (creating an artificial splice site). Use of the miR-E backbone and placement within the intron of the CAG promoter was validated using a model antigen (data not shown).

Construction of dual protein constructs—Codon-optimized genes for Pigment Epithelium Derived Factor (PEDF), VEGF Receptor 3 (VEGFR3)-Fc fusion protein, and anti-Angiopoietin-2 (ANG2) single-chain Fab (scFab) fragments were excised from shuttle vectors and inserted into the AAV vector backbone between the CBA promoter and the SV40 late polyA (SV40 pA) sequence on NheI-MluI fragments. For co-expression with Aflibercept, the synthetic DNA included sequences encoding the C-terminal portion of Aflibercept (AFLB), a Furin cleavage site, and a T2A ribosomal-skipping peptide upstream of the PEDF, VEGFR3-Fc, or anti-ANG2 scFab sequences. These synthetic DNAs were excised from the shuttle vectors and inserted into pAAV-CAG-AFLB-SV40 pA on AvrII-MluI fragments. Plasmids were propagated in E. coli and purified plasmid DNA was verified by restriction digest and sequencing.

Construction Details for Protein+RNAi Constructs

Construction of pAAV-CAG-miR-E-(“Target Sequence”)-AFLH-SV40

The pAAV-CAG-AFLB-SV40 construct expressing human codon optimized Aflibercept was synthesized as described previously. The miR-E-(target) miRNA transgene, encoding for hairpin targeting ANGPT2, VEGF-C, or VEGFR3, containing the region of the CAG beta-actin intron encoded between the SgrAI and NheI restriction cloning sites was synthesized and cloned into pUC57 by Genscript (Genscript, Picataway, NJ). pUC57 plasmid and the pAAV-CAG-AFLB-SV40-Kan-Stuffer plasmid were cut with various restriction enzymes (New England Biolabs) as indicated, backbone DNA was also treated with recombinant shrimp alkaline phosphatase (rSAP, M0371L, New England Biolabs) during digest to remove free phosphates on cut DNA ends. DNA fragments were added at a 7:1 molar ratio insert:backbone and ligated with Quick Ligase per manufacturer's instructions (#M2200L, New England Biolabs). Ligated plasmid was transformed into NEB Stable bacterial competent cells (#C3040H, New England Biolabs) per manufacturer's instructions and the cells were spread on Kanamycin 50 mg/mil plates (#L1025, Teknova, Hollister, CA) and grown at 30° C.

Reparation of pAAV-CAO-miR-E-AFLB-SV40

Miniprep cultures were grown from the resulting colonies, DNA was prepared with the GeneJET Plasmid Miniprep kit (Cat. #0503, ThermoFisher, Waltham, MA) and restriction digested to identify positive clones. A 50-ml culture in Terrific Broth was grown from one positive clone of each construct and DNA was prepared with the Qiagen EndoFree Plasmid Maxi Kit (Cat. #12362, Qiagen, Hilden, Germany).

Restriction Digest and Sequencing of pAAV-CAG-RFP657-miRNA Plasmid Variants

Maxiprep plasmid DNA (0.5 mg) was digested with various restriction enzymes (New England BioLabs) according to the manufacturer's instructions and analyzed by agarose gel electrophoresis. Sanger DNA sequencing was performed by ELIM using primers.

A summary of the constructs designed and tested in the examples herein is provided below:

TABLE 5

| Name | Promoter | Additional Transgene(s)* | RNAi location | Size (bp) |
|---|---|---|---|---|
| | CBA | PEDF (SEQ ID NO: 60) | N/A | 4297 |
| | CBA | anti-Ang-2 scFab LH | N/A | 4645 |
| | CBA | anti-Ang-2 scFab HL | N/A | 4642 |
| pP141.001 | CAG | Ang2-targeting RNAi | Promoter intron | 3863 |
| | | (SEQ ID NOs: 13 and 14) | | |
| | CAG | VEGFR3-targeting RNAi | Promoter intron | 3863 |
| | | (SEQ ID NOs: 37 and 38) | | |
| pP145.001 | CAG | VEGF-C-targeting RNAi | Promoter intron | 3830 |
| | | (SE ID NOs: 19 and 20) | | |
| pP151.001 | CAG | VEGF-C-targeting RNAi | Promoter intron | 4136 |
| | | (SEQ ID NOs: 19 and 20) | | |
| | | Ang-2-targeting RNAi | 3′ UTR (AFLB) | |
| | | (SEQ ID NOs: 13 and 14) | | |
| pP152.001 | CAG | VEGF-C-targeting RNAi | Promoter intron | 4138 |
| | | (SEQ ID NOs: 19 and 20) | | |
| | | Ang-2-targeting RNAi | AFLB coding | |
| | | (SEQ ID Nos: 13 and 14) | sequence | |
| pP153.001 | CAG | VEGF-C-targeting RNAi | Promoter intron | 3987 |
| | | (SEQ ID Nos: 19 and 20) | | |
| | | Ang-2-targeting RNAi | Promoter intron | |
| | | (SEQ ID Nos: 13 and 14) | | |

*each construct comprises nucleotide sequence encoding aflibercept (AFLB)

Aflibercept is expressed in human RPE and RGC cells at therapeutic levels following delivery in rAAV virions comprising a capsid protein of SEQ ID NO:48, resulting in efficient blockade of VEGF-A, VEGF-B and PIGF-mediated activity in these cells. See US Patent Application Publication No. 2020/0282077A1, the contents of which are incorporated herein by reference.

Studies were conducted to assess the effect of including a second transgene (encoding a protein or an RNAi) in AAV expression plasmids on expression of aflibercept following transfection of HEK293T cells.

Briefly, HEK293T cells were seeded in 12-well plates at 2.0×10⁵ cells/well in 1.0 ml DMEM/10% FBS media. The next day, 1.0 mg plasmid DNA (comprising nucleotide sequence encoding aflibercept and a second transgene under the control of the same promoter) complexed with 3.0 ml FuGeneHD (Cat. #E2691, Promega, Madison, WI) was added to the cells in triplicate wells. 48 hrs post-transfection, cell supernatant was harvested and spun @ 2000 g to remove cellular debris. Media was then assayed for the presence of aflibercept (ALFB) via ELISA.

For Western Blot analysis, media from transduced HEK293T cells were pooled from 3 replicates and mixed with 4×LDS (B0007, Thermo), 10× Reducing Agent (B0009, Thermo) and denatured at 70° C. for 10 minutes. Samples were loaded on a 10-well Bolt 4-12% Bis-Tris Plus polyacrylamide gel (Invitrogen, NWO4120BOX) and ran in 1× MOPS buffer (NP000102, Thermo) at 200V for 32 minutes. Separated proteins were transferred to a nitrocellulose filter (1704158, BioRad) with the BioRad TransBlot Turbo device (BioRad) for 7 minutes and probed anti-Human IgG Fc Cross-Adsorbed Secondary Antibody, HRP (ThermoFisher, 31413) at 1:500 in iBind Flex solution (SLF2020, Thermo). Proteins were visualized with SuperSignal West Dura Chemiluminescent Substrate (ThermoFisher 34076) and imaged on a ChemiDoc MP (BioRad, Hercules, CA).

Cell lysates for ELISA (secreted free-AFLB, ANGPT2, and VEGF-A Levels) were prepared in M-PER lysis buffer (#78501, Thermo) supplemented with 1× Halt Protease and Phosphatase Inhibitor Cocktail (78440, Thermo) as per manufacturer's instructions. Cell media and lysate were diluted appropriately for each sample and were used to evaluate secreted analyte levels using the Aflibercept ELISA kit (to measure free AFLB levels) (Cat. #IG-AA115, Eagle Biosciences, Nashua, NH), the Quantikine human VEGF-A ELISA kit (DVE00, R&D Systems) and the Quantikine human ANGPT2 ELISA kit (DANG20, R&D Systems) following the provider's instructions. The optical density (OD) was measured with a Cytation 3 (BioTek, Winooski, VT) photometer at 450 nm (reference at OD 620 nm) within 15 min after pipetting the Stop Solution. Media concentrations were defined based on the generated standard curve.

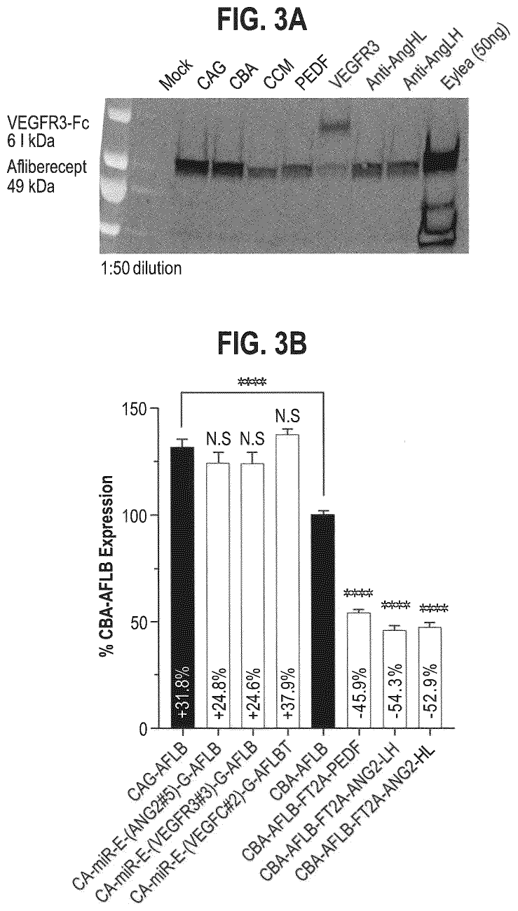

FIG. 3A demonstrates the results of Western Blot analysis of AFLB from media of HEK293T cells following transfection with AAV plasmids encoding AFLB and a second transgene (PEDF, VEGFR3, Anti-Ang HL, or Anti-AngLH) under the control of a CAG promoter. Results are normalized to AFLB level in media of HEK293T cells transfected with AAV plasmid encoding only AFLB under the control of CBA promoter. Elyea (commercial preparation) is provided as a positive control for aflibercept. Addition of a second transgene encoding a polypeptide product significantly reduces expression of aflibercept by ˜50-60%.

FIG. 3B demonstrates expression of aflibercept from various constructs encoding either a second polypeptide (PEDF, anti-Ang2 LH, anti-Ang2 HL) or interfering RNA (targeting Ang2, FEGR3 or VEGF-C) normalized to AFLB level in media of HEK293T cells transfected with AAV plasmid encoding only AFLB under the control of CBA promoter. Notably, no significant reduction in aflibercept expression occurs with plasmids encoding AFLB and an interfering RNA. Expression from the CAG promoter was observed to be 25% greater than expression from the CBA promoter.

Next, studies were conducted to assess the effect of including a second transgene (encoding a protein or an RNAi) in AAV expression plasmids on expression of aflibercept following transduction of human retinal pigment epithelium (RPE) cells with recombinant AAV virus comprising a capsid protein of SEQ ID NO:48.

For RPE transduction, human stem cell derived retinal pigment epithelial cells (RPE) were differentiated from embryonic stem cells (ESI-017) following published protocols (Buchholz D 2013, Leach L 2015). RPE cells were grown on Matrigel (Corning) for 30 days in XVIVO-10 media (Lonza), in a 96 well plate format. Prior to transduction, three wells were harvested and counted for an accurate calculation of multiplicity of infection (MOI). Virus was added to the cells for 48 hours in XVIVO-10 media based on each viral titer in a total volume of 100 μL per well. Media was collected on day 3, 7, 11, 15 and 19 and replaced with 200 μL of media per well. Media samples were stored at 4° C. until processed.

Cell lysates for ELISA (to assess secreted free-AFLB, ANGPT2, and VEGF-A levels) were prepared in M-PER lysis buffer (#78501, Thermo) supplemented with 1× Halt Protease and Phosphatase Inhibitor Cocktail (78440, Thermo) as per manufacturer's instructions. Cell media and lysate were diluted appropriately for each sample and were used to evaluate secreted analyte levels using the Aflibercept ELISA kit (to measure free AFLB levels) (Cat. #IG-AA115, Eagle Biosciences, Nashua, NH), the Quantikine human VEGF-A ELISA kit (DVE00, R&D Systems) and the Quantikine human ANGPT2 ELISA kit (DANG20, R&D Systems) following the provider's instructions. The optical density (OD) was measured with a Cytation 3 (BioTek, Winooski, VT) photometer at 450 nm (reference at OD 620 nm) within 15 min after pipetting the Stop Solution. Media concentrations were defined based on the generated standard curve.

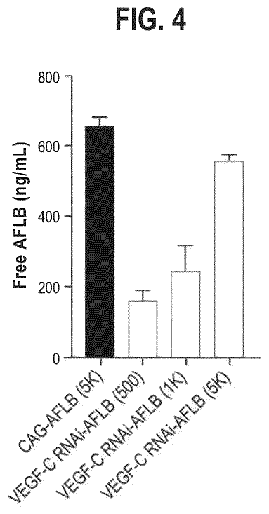

As can be seen from FIG. 4, a dose-dependent increase in aflibercept expression is seen (day 8 is shown) with rAAV expressing aflibercept and an RNAi targeting VEGF-C, with the high multiplicity of infection (MOI) near that of the control (rAAV expressing aflibercept alone operably linked to CAG promoter). All constructs expressing aflibercept are able to completely neutralize endogenous VEGF-A at all multiplicities of infection (MOIs) tested. The amounts shown in FIG. 4 are free and active aflibercept in the media of RPE cells following transduction.

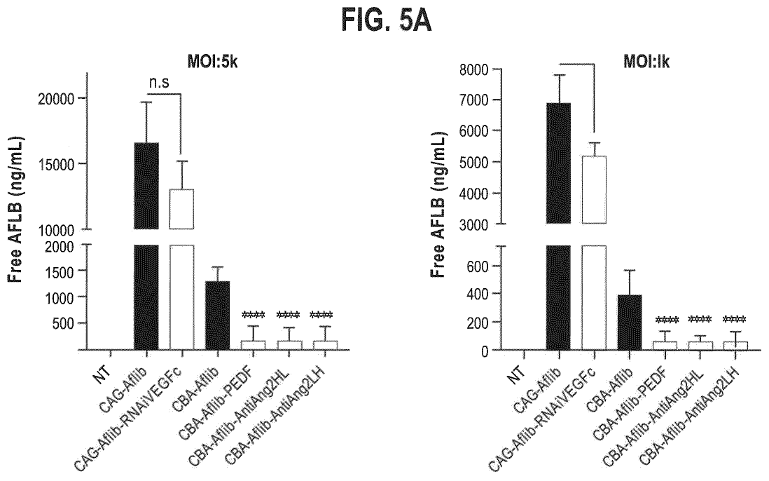

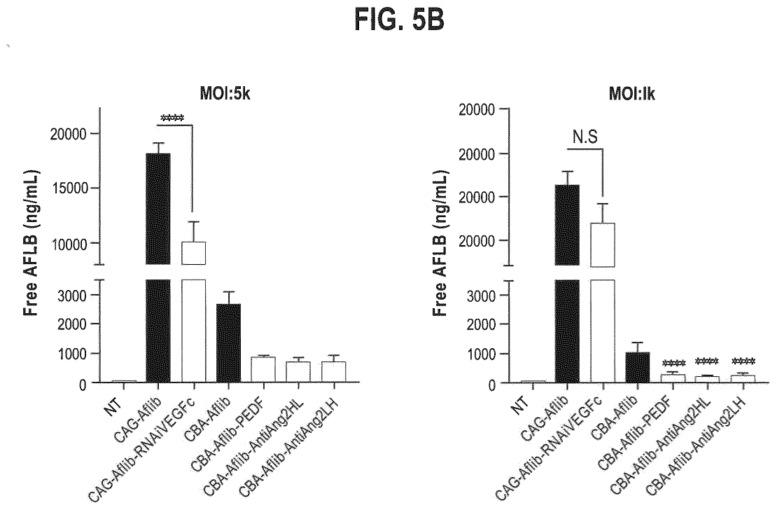

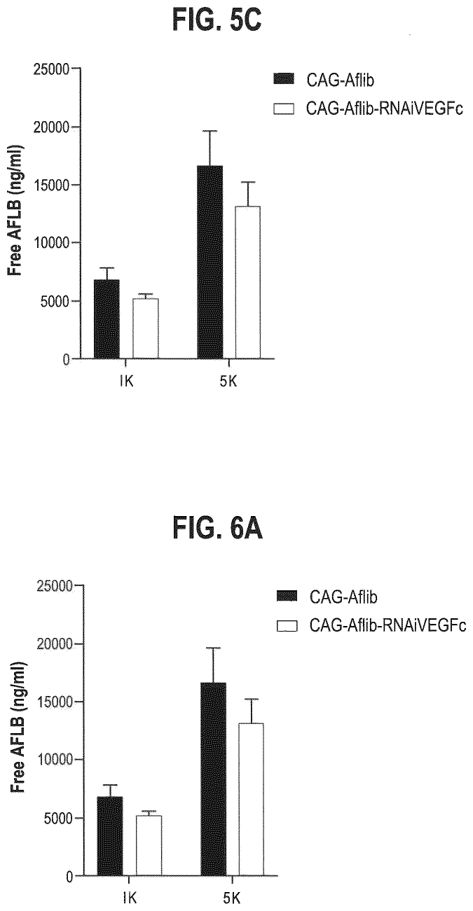

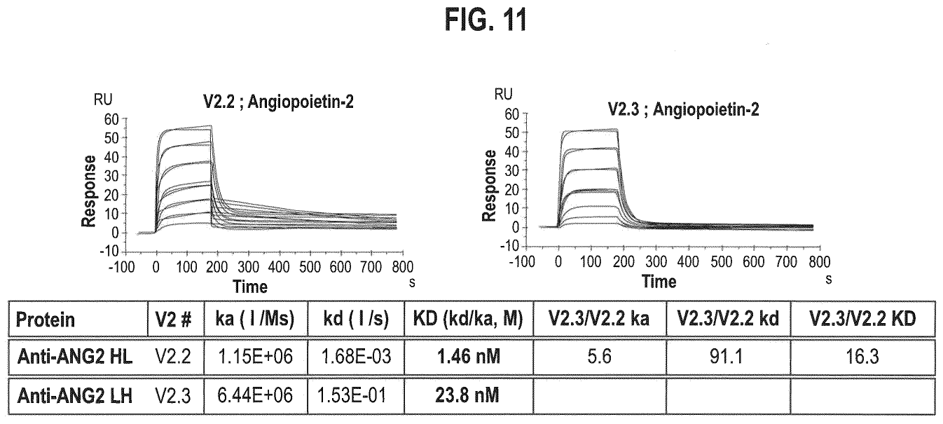

FIGS. 5A and 5B illustrate analysis of free aflibercept in RPE supernatant on days 7 (FIG. 5A) and 11 (FIG. 5B) following transduction with rAAV carrying the indicated expression cassettes (and a capsid protein of SEQ ID NO:48). As in HEK293T cells, expression of a second protein dramatically reduces the level of aflibercept expression in transduced RPE cells. In contrast, while some reduction in aflibercept expression was observed with co-expression of interfering RNA, aflibercept expression remained robust at both MOIs. The CBA promoter was surprisingly determined to be much weaker than the CAG promoter at driving expression in RPE cells. See also FIGS. 6A-B illustrating some decrease in aflibercept expression at the higher MOI with co-expression of interfering RNA but much less pronounced than with co-expression of a second polypeptide.

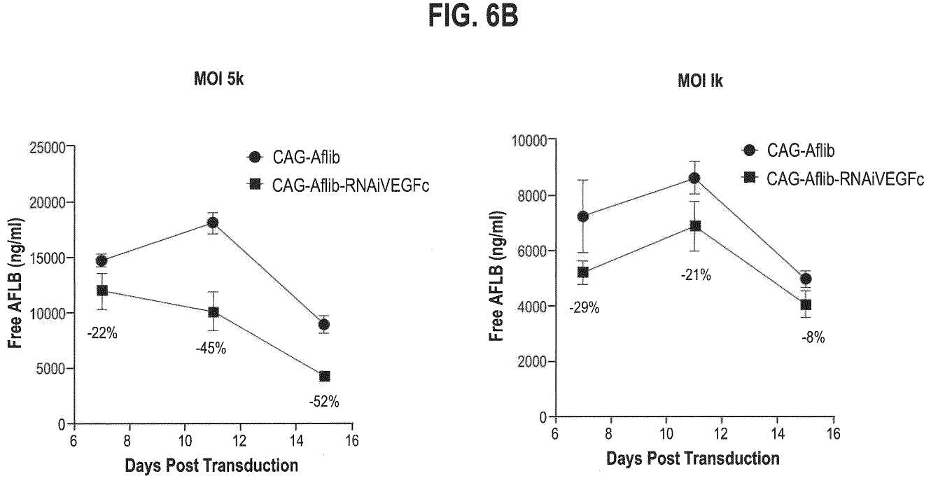

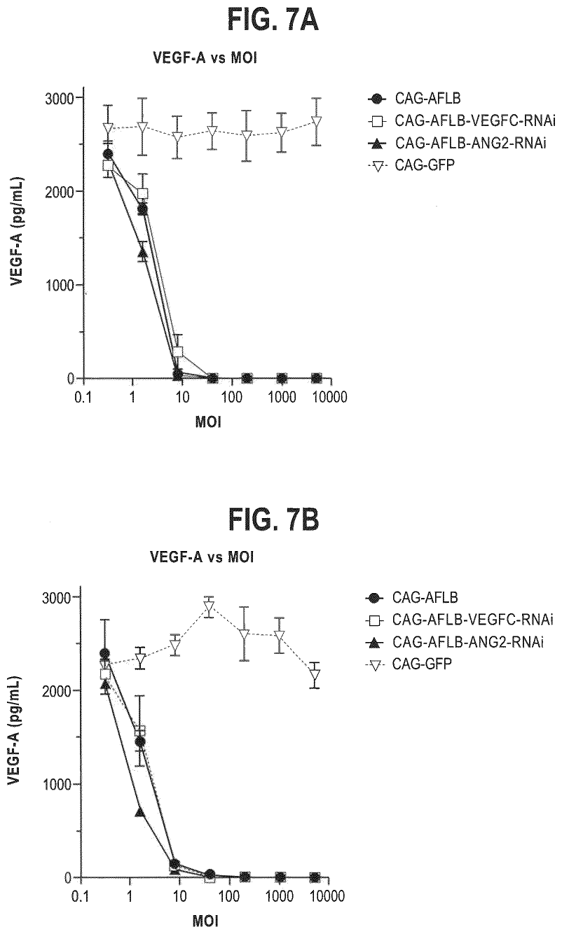

Importantly, aflibercept co-expressed with interfering RNA was functional and able to bind VEGF-A produced by RPE cells (see FIGS. 7A-B illustrating neutralization of VEGF-A at MOIs above 40 for AFLB+VEGFC-RNAi and AFLB+ANG2 RNAi constructs).

### CONCLUSIONS

In HEK293T cells, aflibercept expression is reduced ˜20% when driven by the CBA promoter compared to the CAG promoter; expression of aflibercept was ˜13× weaker when driven by the CBA promoter compared to the than the CAG promoter in RPE cells.

In HEK293T cells, RNAi has no significant effect on transgene expression compared to control (aflibercept alone under the control of the same promoter), whereas all dual protein constructs exhibited a ˜50% reduction in aflibercept compared to control (aflibercept alone under the control of the same promoter) in HEK293T cells. In RPE cells, under most conditions a slight, but insignificant, reduction in free aflibercept levels was observed compared to control in RPE cells for constructs containing RNAi, whereas all dual protein constructs expressed ˜5-10× less aflibercept than their control counterpart.

### Example 2—Characterization of Dual Protein Constructs

Characterization of Constructs Expressing Aflibercept+Anti-Ang-2 scFab

HEK293T cells were transfected with AAV plasmid comprising nucleotide sequence encoding aflibercept and nucleotide sequence expressing anti-Ang-2 scFab in a bicistronic configuration driven by the CAG promoter (see FIG. 1A). Briefly, HEK293T cells were seeded in 12-well plates at 2.0×10⁵ cells/well in 1.0 ml DMEM/10% FBS media. The next day, 1.0 mg plasmid DNA complexed with 3.0 ml FuGeneHD (Cat. #E2691, Promega, Madison, WI) was added to the cells in triplicate wells. 48 hrs post-transfection, cell supernatant was harvested and spun a, 2000 g to remove cellular debris. Media was then assayed for the presence of ALFB via ELISA.

Western Blot—media from transfected HEK293T cells (6.25 μl) was mixed with 12.5 μl 4×LDS, 5 μl 10× Reducing Agent, and 26.25 1×PBS (final volume=50 μl) and denatured at 70° C. for 10 minutes. 40 μl of the samples were loaded on a 10-well Bolt 4-12% Bis-Tris Plus polyacrylamide gel (Invitrogen, NWO4120BOX) and ran in 1× MOPS buffer at 200V for 32 minutes. Separated proteins were transferred to a nitrocellulose filter with the iBlot 2 device (ThermoFisher) for 7 minutes and probed with anti-Human IgG F(ab′)₂ Secondary Antibody (ThermoFisher 31482 1:1000) using the iBind Flex device (ThermoFisher). Proteins were visualized with SuperSignal West Dura Chemiluminescent Substrate (Thermofisher 34076) and imaged on a ChemiDoc MP (BioRad, Hercules, CA).

Functional anti-ANG2 ELISA with ANG2 coated plates—Nunc MaxiSorp flat-bottom plates (Invitrogen, 44-2404-21) were coated with 100 μl of 1.0 μg/μl Recombinant Human Angiopoietin-2 (R&D, 623-AN/CF) in PBS, sealed with an adhesive sheet, and placed at 4° C. overnight. The next day, the coating solution was aspirated, and the plates were washed 3 times with 300 μl PBST (PBS/0.05% Tween 20). The plates were blocked with 200 μl PBS/2.0% BSA at room temperature for 2 hours. After the 2-hour incubation, the blocking solution was aspirated, and the plate was washed 3 times with 300 μl PBST. Media from transfected HEK293T cells was diluted in PBS/0.2% BSA, and 100 pd of the diluted media was added to the plate and incubated at room temperature for 2 hours with gentle shaking. The plate was washed again 3 times with 300 μl PBST. 100 μl of anti-Human IgG F(ab′)₂ Secondary Antibody (ThermoFisher 31482, 1:20,000) was added to the plate and incubated at room temperature for 1 hour with gentle shaking. The plate was washed again 3 times with 300 μl PBST. The plates were developed with 100 μl TMB ELISA Substrate (Abcam, ab171522) at room temperature for 5-15 minutes. The TMB reaction was stopped with 100 μl 450 cun Stop Solution for TMB Substrate (Abcam, ab171529). The plates were read at 450 nm and 540 nm (as a reference blank for the plate) using the Cytation 5 device (BioTek).

ANG2 Competition ELISA—media from transfected HEK293T cells was diluted in PBS/0.2% BSA, mixed with an equal volume of 2.0 ng/ml Recombinant Human Angiopoietin-2 (R&D, 623-AN/CF), and incubated overnight at room temperature (final ANG2 concentration=1,000 μg/ml). The next day, the concentration of free ANG2 was determined with the Angiopoietin-2 Human ELISA Kit (Invitrogen, KHC1641) as interpolated from a freshly made Recombinant Human Angiopoietin-2 (R&D, 623/AN-CF) standard.

ANG2 Receptor Competition Assay—nunc MaxiSorp flat-bottom plates (Invitrogen, 44-2404-21) were coated with 100 μl of 1.0 μg/μl Recombinant Human Tie-2 Fc Chimera Protein (R&D, 313-TI/CF) in PBS, sealed with an adhesive sheet, and placed at 4° C. overnight. The competition mix was prepared using dilutions of media from transfected HEK293T cells with an equal volume of 80 ng/ml Human Angiopoietin-2 with an N-terminal FLAG-tag (Adipogen, AG-40B-0114-C010) (final ANG2-Flag concentration=80 ng/ml). The next day, the coating solution was aspirated, and the plates were washed 3 times with 300 μl PBST. The plates were blocked with 200 μl PBS/2.0% BSA at room temperature for 2 hours. After the 2-hour incubation, the blocking solution was aspirated, and the plate was washed 3 times with 300 μl PBST. 100 μl of the competition mix samples along with a freshly made ANG2-Flag standard were added to the plate and incubated at room temperature for 2 hours with gentle shaking. The samples in the plate were aspirated and washed 3 times with 300 μl PBST. 100 μl of DYKDDDDK (SEQ ID NO:71) Epitope Tag Horseradish Peroxidase-conjugated antibody (R&D HAM85291 1:10000) was added to the plate and incubated at room temperature for 1 hour with gentle shaking. The plate was washed 3 times with 300 μl PBST and developed with 100 μl TMB ELISA Substrate (Abcam, ab171522) at room temperature for 5-15 minutes. The TMB reaction was stopped with 100 μl 450 nm Stop Solution for TMB Substrate (Abcam, ab171529). The plates were read at 450 nm and 540 nm (as a reference blank for the plate) using the Cytation 5 device (BioTek) and the concentration of ANG2-Flag was interpolated from the ANG2-Flag standard.

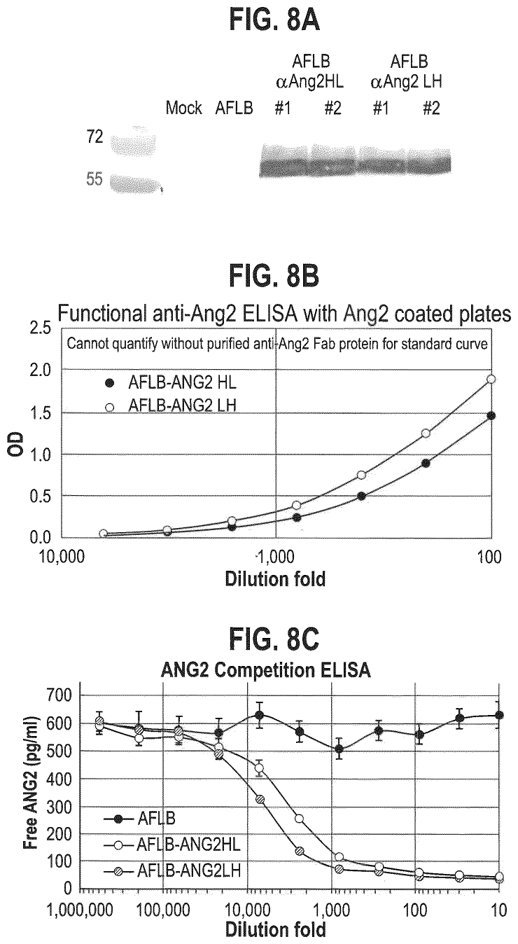

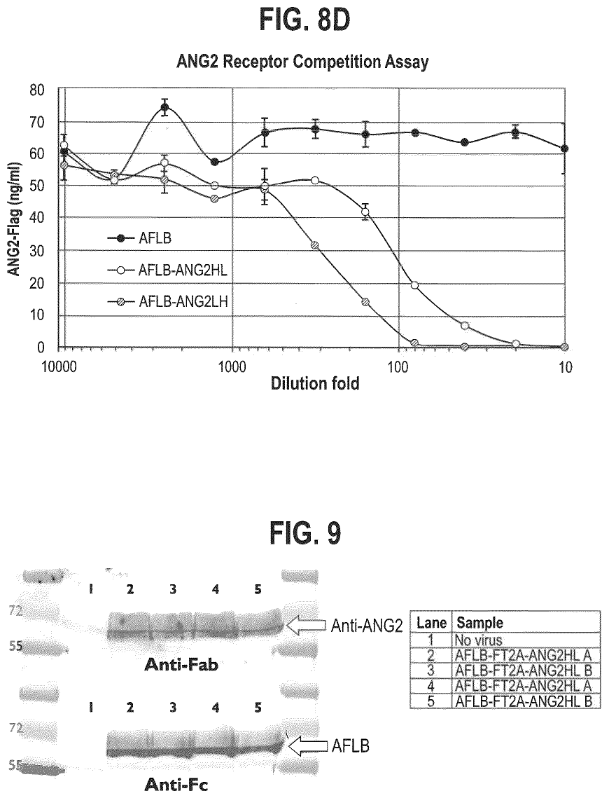

FIG. 8A illustrates expression of protein products of the expected size for anti-Ang-2 HL and HL Fab. FIG. 8B illustrates that expressed anti-Ang-2 Fab is functional (binding of expressed protein to Ang-2-coated plates is shown). FIG. 8C illustrates that expressed anti-Ang-2 Fab blocks binding of Ang-2 to antibody-coated plates (competition ELISA). FIG. 8D illustrates that expressed anti-Ang-2 Fab blocks binding of Ang-2 to Tie-2 (receptor competition assay).

Dose-dependent expression of anti-Ang-2 Fab (LH and HL formats) from dual protein construct was shown from transfected HEK cells. The expressed anti-Ang-2 Fab proteins were the correct size and functional at binding Ang2 and blocking Ang2-binding to its receptor.

Next, studies were conducted to characterize expression of aflibercept and anti-Ang-2 Fab following transduction of human retinal pigment epithelium (RPE) cells with recombinant AAV virus comprising a capsid protein of SEQ ID NO:48 and a nucleic acid encoding aflibercept and anti-Ang-2 Fab (LH or HL).

FIG. 9 illustrates expression of a single protein of the correct size (53 kD) for Anti-Ang-2 scFab (LH and HL), with similar expression levels of LH and HL conformations. Briefly, RPE cells were transduced with rAAV comprising a capsid protein of SEQ ID NO:48 and a nucleic acid encoding aflibercept and anti-Ang-2 scFab at MOI 5,000 and 1,000 in 96 well plates. Media (0.2 ml) was changed at day 3 post-transduction and collected on day 7. 25 ml media of the MOI 5,000 samples was run on 4-12% SDS-PAGE, transferred to nitrocellulose with iBlot and probed on the iBind Flex with HRP-conjugated anti-Hu-Fab antibody to detect the anti-Ang-2 scFab and anti-Hu-Fc antibody to detect Aflibercept.

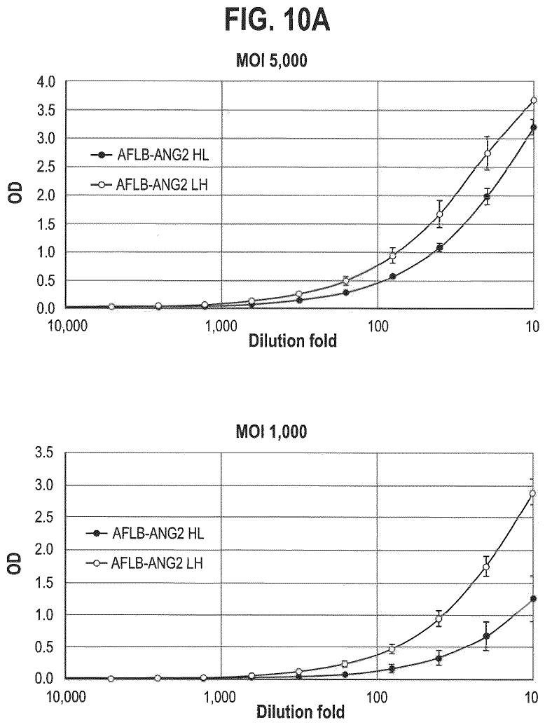

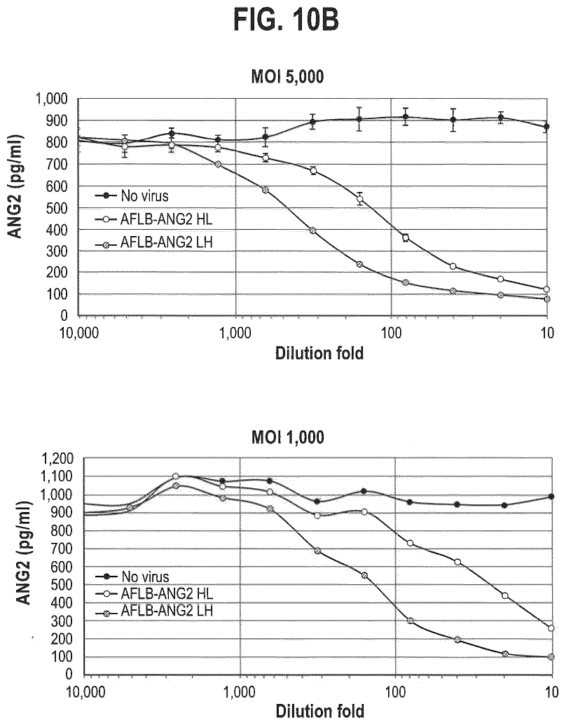

FIG. 10A illustrates dose-dependent expression of functional Anti-Ang-2 scFab in transduced RPE cells at MOI of 1,000 and 5000. Briefly, dilutions of media from transduced RPE cells (day 11) were incubated on Ang-2-coated plates. Anti-Ang-2 scFab bound to Ang-2 was detected with HRP-conjugated anti-Hu-Fab antibody. The LH format was slightly more effective at binding Ang2 than the HL format, especially at lower MOT.

FIG. 10B illustrates the results of competition ELISA with day 11 samples in transduced RPE. Briefly, 1000 μg/ml Ang2 protein was incubated overnight with a dilution series of media from transduced RPE cells. Competition mixtures were assayed by Ang-2 ELISA (Invitrogen KHC1641).

Next, binding affinities of anti-Ang-2 scFab LH and HL were compared via SPR by Biacore assay performed at Genscript.

Immobilization of Angiopoietin-2 onto CM5 sensor chip. The immobilization of Angiopoietin-2 was performed under 25 degrees Celsius while HBS-EP+ was used as the running buffer. The sensor chip surface of flow cells 1, 2 were activated by freshly mixed 50 mmol/L N-Hydroxysuccinimide (NHS) and 200 mmol/L 1-ethyl-3-(3-dimethylaminopropyl) carbodiimide hydrochloride (EDC) for 420 s (10 μL/min). Afterwards, Angiopoietin-2 diluted in 10 mmol/L NaAC (pH 4.5) were injected into the flow cell 2 to achieve conjugation of 243.1 Response Unit. After the amine coupling reaction, the remaining active coupling sites on chip surface were blocked with 420 s injection of 1 mol/L ethanolamine hydrochloride. For affinity measurement, the assay was performed at 25° C. and the running buffer was HBS-EP+. Diluted V2.2/V2.3 were injected over the surface of flow cell 1, 2 as association phase, followed by injecting running buffer as dissociation phase. Running configuration was listed below (Sample concentrations (nM)=1.5625, 3.125, 6.25, 12.5, 25, 50, 100)

| Immobilization | Ligand = | Immobilization | Flow rate |
|---|---|---|---|
| | Angiopoietin 2 | level (RU) = | (ml/min) = 10 |
| | | 234.1 | |
| Association & | Association | Dissociation | Flow rate |
| Dissociation | contact time = | contact time = | (ml/min) = 30 |
| | 180 | 600 | |

The results are provided at FIG. 11. The LH configuration (V2.3) has a 5.6-fold faster association (higher ka value) than the HL configuration (V2.2) suggesting stronger binding. The LH configuration has a 91-fold faster dissociation (higher kd value) than HL suggesting weaker binding. The HL configuration has an overall binding affinity of 1.46 nM and the LH has a binding affinity of 23.8 nM. Thus, LH configuration has about 16-fold higher Ang-2-binding affinity than the HL configuration. These values are consistent with the published affinity for the anti-Ang2 arm of faricimab.

Next, PEDF expression in HEK293T cells was assessed following transfection with AAV plasmid dual protein construct encoding aflibercept and PEDF. Briefly, HEK293T cells were seeded in 12-well plates at 2.0×10⁵ cells/well in 1.0 ml DMENV10% FBS media. HEK293T cells were used due to their high transfectability and protein expression. The next day, 1.0 mg plasmid DNA complexed with 3.0 ml FuGene6 (Cat. #E2691, Promega, Madison, WI) was added to the cells in duplicate wells. Two days after transfection, the supernatants were collected. Cell debris were pelleted by centrifugation in a microcentrifuge at 12,000 g for 10 minutes at 4° C. The supernatant was collected and stored at 4° C. A no plasmid condition was included in the transfection as negative control.

For SDS-PAGE and Western Blot, PEDF samples were diluted 1:10, prior to running the SDS-PAGE/Western blot assay. Aflibercept samples were diluted 1:50. Diluted media was combined with 4× sample buffer and 10× reducing buffer according to the ThermoFisher iBlot system. Samples were then boiled at 90° C. for 5 minutes. A 4-12% Bis/Tris reducing gel was run in 1× MOPS buffer at 125V for 1.5 hours. The proteins were transferred onto nitrocellulose using the BioRad Trans Blot Turbo System using the preset MIXED protocol for 7 minutes. Membranes were blocked for 1-2 minutes in iBind Flex Buffer prior to loading the iBind Flex Western Device according to manufacturer's instructions. Primary antibodies (anti-PEDF EMD Millipore, 1:1000, anti-Human FC HRP ThermoFisher 1:2000) with species specific secondary antibodies conjugated to HRP (1:10000) were used and detected with Femto ECL Substrate and imaged on a BioRad ChemiDoc system.

ELISA (R&D, Human serpin F1/PEDF ELISA Cat. No. DY1177) was carried out according to manufacturer's instructions. Media was diluted 1:1000 or 1:10000 prior to running the assay.

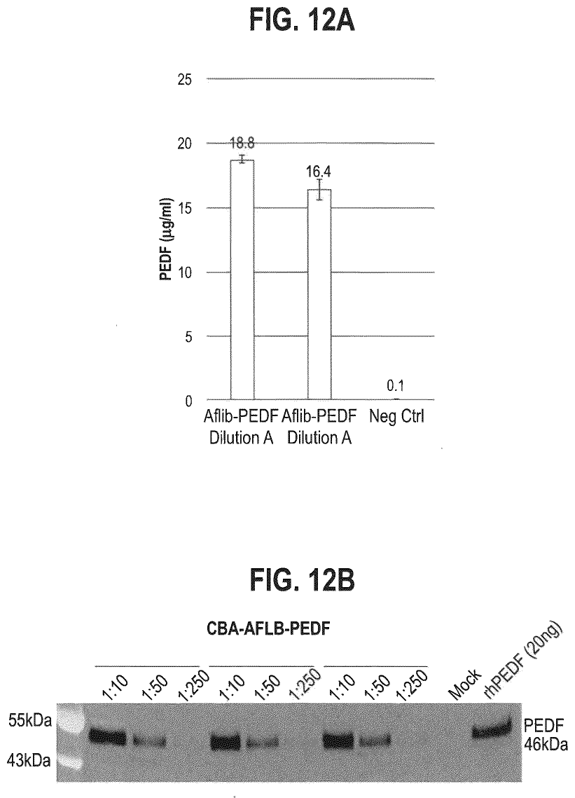

FIGS. 12A-B illustrate expression of a single protein product of the expected size for PEDF from transfected HEK293T cells, with production of ˜17 mg/ml PEDF.

VEGFR3-Fc expression in HEK293T cells was assessed following transfection with AAV plasmid dual protein construct encoding aflibercept and VEGFR3-Fc. Briefly, HEK293T cells were seeded in 12-well plates at 2.0×10⁵ cells/well in 1.0 ml DMEM/10% FBS media. HEK293T cells were used due to their high transfectability and protein expression. The next day, 1.0 mg plasmid DNA complexed with 3.0 ml FuGene6 (Cat. #E2691, Promega, Madison, WI) was added to the cells in duplicate wells. Two days after transfection, the supernatants were collected. Cell debris were pelleted by centrifugation in a microcentrifuge at 12,000 g for 10 minutes at 4° C. The supernatant was collected and stored at 4° C. A no plasmid condition was included in the transfection as negative control.

For reducing Western Blotting, cell soups (20 ml) was mixed with 10 ml 4×LDS, 4 ml 10× Reducing Agent, and 6 ml water (final volume=40 ml) and denatured at 70° C. for 10 minutes. Samples were loaded on a 12-well Bolt 4-12% Bis-Tris Plus polyacrylamide gel (Invitrogen, NW04122BOX) and ran in 1× MOPS buffer at 100 V for 75 minutes. Separated proteins were transferred to a nitrocellulose filter with the iBlot 2 device (ThermoFisher) for 10 minutes and probed with HRP-conjugated goat anti-Human IgG Fc (ThermoFisher Scientific, Cat. #31413, 1:2000) using the iBind Flex device (ThermoFisher). Proteins were visualized with SuperSignal West Dura Chemiluminescent Substrate (ThermoFisher 34076) and imaged on a ChemiDoc MP (BioRad, Hercules, CA).

For non-reducing Western Blotting, cell supernatant (30 ml) was mixed with 10 ml 4×LDS (final volume=40 ml) and denatured at 70° C. for 10 minutes. Samples were loaded on a 12-well Bolt 4-12% Bis-Tris Plus polyacrylamide gel (Invitrogen, NW04122BOX) and ran in 1× MOPS buffer at 100 V for 120 minutes. Separated proteins were transferred to a nitrocellulose filter with the iBlot 2 device (ThermoFisher) for 10 minutes and probed with HRP-conjugated goat anti-Human IgG Fc (ThermoFisher Scientific, Cat. #31413, 1:2000) using the iBind Flex device (ThermoFisher). Proteins were visualized with SuperSignal West Dura Chemiluminescent Substrate (ThermoFisher 34076) and imaged on a ChemiDoc MP (BioRad, Hercules, CA).

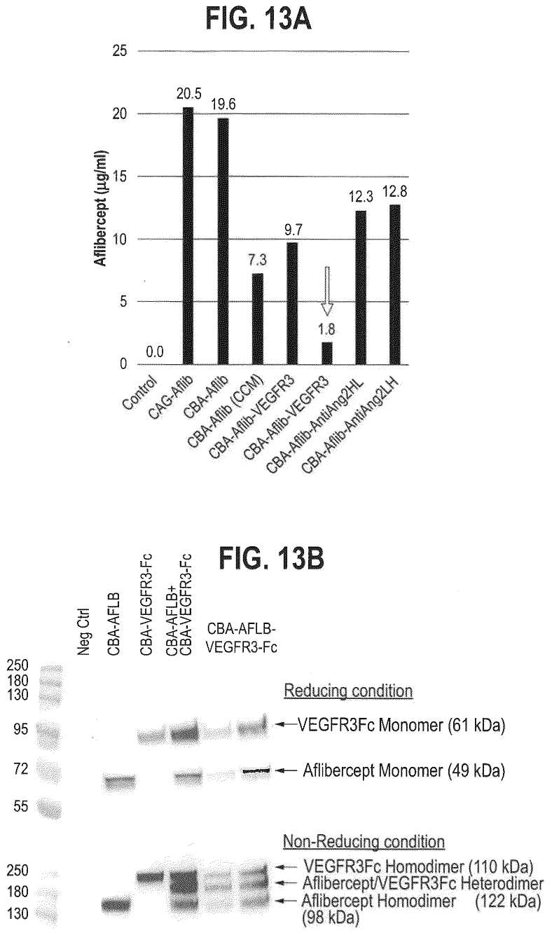

FIG. 13A compares expression of aflibercept following transfection of HEK293T cells with dual protein AAV constructs also encoding PEDF, VEGFR3-Fc, Anti-Ang-2 HL or Anti-Ang-2 LH. FIG. 13B illustrates a dramatic reduction in aflibercept when co-expressed with VEGFR3-Fc due to formation of VEGFR3-Fc/Aflibercept heterodimers

### Example 3—Characterization of Constructs Encoding Aflibercept+RNAi

The general strategy employed was to (i) design shRNA against human targets (ii) select shRNA sequence based on (a) knockdown of endogenous expression of target using lentivirus and (b) homology to NHP sequences (iii) embed miR-E containing sequences from selected shRNA within intron of CAG in anti-VEGF-expressing plasmids or in combination with RFP to assess RNAi function alone.

Ang2 and VEGFR3 are both expressed by endothelial cells—transduction of endothelial cells by rAAV comprising a capsid protein of SEQ ID NO:48 was confirmed.

Characterization of Constructs Comprising RNAi Targeting Ana-2

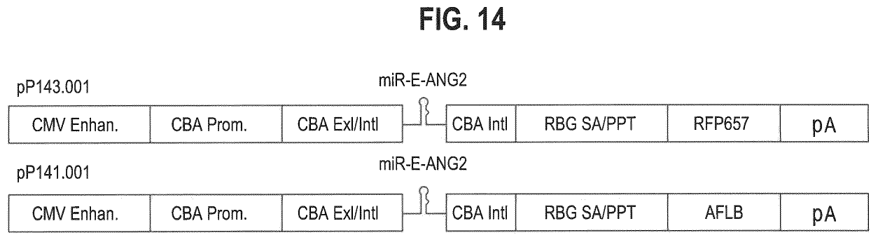

Construction of pAAV-CAG-miR-E (Ang-2)-AFLB-SV40 (see FIG. 14; constructs pP143.001 encodes RFP675 and pP141.001 encodes aflibercept but are otherwise identical; pP143.001 is an miRNA only efficacy control for in vitro studies). The pAAV-CAG-AFLB-SV40 construct expressing human codon optimized Aflibercept, VEGF-A/B Trap was synthesized. The miR-E-(ANG-2) miRNA transgene containing the region of the CAG beta-actin intron encoded between the SgrAI and NheI restriction cloning sites was synthesized and cloned into pUCS7 by Genscript (Genscript, Picataway, NJ). pUC57 plasmid and the pAAV-CAG-AFLB-SV40-Kan-Stuffer plasmid were cut with various restriction enzymes (New England Biolabs) as indicated, backbone DNA was also treated with recombinant shrimp alkaline phosphatase (rSAP, M0371L, New England Biolabs) during digest to remove free phosphates on cut DNA ends. DNA fragments were added at a 7:1 molar ratio insert:backbone and ligated with Quick Ligase per manufacturer's instructions (#M2200L, New England Biolabs). Ligated plasmid was transformed into NEB Stable bacterial competent cells (#C3040H, New England Biolabs) per manufacturer's instructions and the cells were spread on Kanamycin 50 mg/ml plates (#L1025, Teknova, Hollister. CA) and grown at 30° C.

Miniprep cultures were grown from the resulting colonies (7.1), DNA was prepared with the GeneJET Plasmid Miniprep kit (Cat. #0503, ThermoFisher, Waltham, MA) and restriction digested to identify positive clones. A 50-ml culture in Terrific Broth was grown from one positive clone and DNA was prepared with the Qiagen EndoFree Plasmid Maxi Kit (Cat. #12362, Qiagen, Hilden, Germany). Maxiprep plasmid DNA (0.5 mg) was digested with various restriction enzymes (New England BioLabs) according to the manufacturer's instructions and analyzed by agarose gel electrophoresis. Sanger DNA sequencing was performed by ELIM using primers.

Several shRNA sequences targeting Ang-2 (the five sequences listed in Table 1) were evaluated for their ability to reduce expression of Ang-2 in HUVEC cells. Briefly, pooled Human umbilical vein endothelial cells were sourced from Lonza (Catalog #C2519A) and cultured in Lonza Endothelial Cell Growth medium (EGM-2, Catalog #: CC-3162) according to manufacturer instructions. HUVECs were passaged using PBS (without Ca and Mg), Trypsin 0.05% and Defined trypsin inhibitor. HUVECs were seeded into plastic (uncoated) cell cultureware at a density of 2,500 cells per cm². Assays were typically performed in 24-well cell culture plates with a 0.5 mL volume of EGM-2, refreshed every other day. HUVECs were typically used only before passage 8, upon which a new culture would be initiated.

HUVEC Transduction—HUVECs were seeded into assay plates at 2,500 cells per cm². After two days, cells were confluent and a single well was dissociated and counted. Multiplicity of infection was calculated using qPCR-derived viral titer and the cell count. The appropriate volume of AAV was applied to the cells in 0.5 mL EGM-2 media. This was incubated for 48 hours and final assay takedown was performed at one week post-transduction.

Generation of ANG2 shRNA Lentiviral Lines—pLKO.1-shANG2 plasmids were generated by ligation of annealed phosphorylated oligos, corresponding to 0.5 unique target sequences in human ANG2 identified from the Broad RNAi consortium (Table 1), into the pLKO.1 vector (Sigma Aldrich, Cat #: SCH001) via EcoRI and AgeI (New England Biolabs) restriction cloning. Plasmid was confirmed by sequencing as with AAV vectors. Maxi prep DNA was generated as with AAV vectors. HEK293T cells were seeded in 6-well plates at 5.0×10⁵ cells/well in 2.0 ml DMEM/10% FBS media. The next day, 0.5 ug pSF-GFP plasmid DNA, 4.6 uL of MISSION Lentiviral Packaging Mix (Sigma Aldrich, Cat #: SHP001) complexed with 2.7 ul FuGene6 (Cat. #E2691, Promega, Madison, WI) was added to the cells. The next day, the media was replaced with 2 mL of fresh media. On the two subsequent days, media containing lentivirus was collected and replaced on the cells with fresh media. Supernatant was collected and filtered with a 0.45 um syringe filter (Millipore Sigma, Cat #SLHVM33RS), aliquoted and stored at −80° C. Lentivirus was titered using the Lenti-X™ qRT-PCR Titration Kit via qPCR manufacturer's instructions (Takara, Cat #631235). Human umbilical vein endothelial cells (HUVECs) (#PCS-100-013, ATCC) were seeded at 2.0×10⁵ cells/well in 6 well plates in 2 ml complete EGM-2 media (CC-3162, Lonza). Immediately after plating, cells were transduced with lentivirus at a multiplicity of infection (MOI) of 25 viral genomes per cell. After 48 hrs, media containing lentivirus was removed and replaced with fresh media containing 1.5 ug/mL puromycin (10 mg/ml stock solution, Sigma Aldrich, Cat #: P9620-10 ml). After 72 hrs of selection with puromycin, media containing dead-uninfected cells was removed. Cells expressing shRNA were continually cultured in 1.0 ug/mL puromycin in all experiments to retain shRNA expression. Media and lysate of infected HUVECs was analyzed by ELISA for ANGPT2

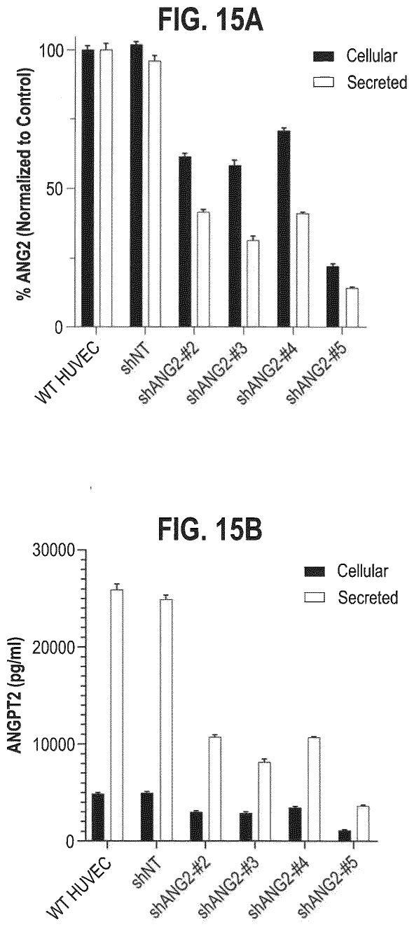

FIGS. 15A-B illustrate the percent of Ang-2 (secreted and cellular) in HUVEC cells following transduction with lentivirus containing each of the candidate shRNAs. All constructs worked well (>50% KD in secreted Ang-2), with shRNA #5 exhibiting the greatest reduction of Ang-2 protein from HUVECs. Notably, shRNAs #1, #4, and #5 are perfect matches in non-human primates. shRNA #5 was selected for inclusion into AAV plasmid constructs encoding aflibercept and miRNA targeting Ang-2. Ang-2 shRNAs #1-5 comprise the following sense and antisense strand sequences:

| shRNA | | |
|---|---|---|
| Sequence | | |
| # | Sense Strand Sequence | Antisense Strand Sequence |
| 1 | GCCGCAGCCTATAACAACTTT | AAAGTTGTTATAGGCTGCGGC |
| | (SEQ ID NO: 1) | (SEQ ID NO: 2) |
| 2 | CCCTAATTCTACAGAAGAGAT | ATCTCTTCTGTAGAATTAGGG |
| | (SEQ ID NO: 4) | (SBQ ID NO: 5) |
| 3 | GATGATAGAAATAGGGACAAA | TTTGTCCCTATTTCTATCATC |
| | (SEQ ID NO: 7) | (SEQ ID NO: 8) |
| 4 | GCCACGGTGAATAATTCAGTT | AACTGAATTATTCACCGTGGC |
| | (SEQ ID NO: 10) | (SEQ ID NO: 11) |
| 5 | GCTTACTCATTGTATGAACAT | ATGTTCATACAATGAGTAAGC |
| | (SEQ ID NO: 13) | (SEQ ID NO: 14) |

Next, human retinal microvascular endothelial cells were transduced with rAAV comprising a capsid protein of SEQ ID NO:48 and a nucleic acid encoding miRNA comprising the sense and antisense strands of shRNA #5 (the sense and antisense strands of shRNA #5 were embedded within mir-E and the miRNA was placed within the hybrid intron of the CAG promoter). rAAV comprising a capsid protein of SEQ ID NO:48 and a nucleic acid encoding GFP under the control of CAG promoter was used as a control.

Briefly, human retinal microvascular endothelial cells were purchased from Cell Systems (Catalog number ACBRI 181) along with “The System” (catalog number CSS-A101). which contains media, coating matrix, and passaging reagents. All passaging, cryopreservation, and cell thawing were performed according to manufacturer's instructions. Cultures are vialed by Cell Systems at passage 3 and, upon receipt, this vial was expanded and cryopreserved as a bank. Experiments were performed on cultures only before passage 9. Experiments were typically performed in 24-well plates, passaged at a 1:3 ratio (1E+4 cells per cm². The media volume was 1 mL per well and media replenished every other day until passage.

For RMVEC transduction, RMVEC were seeded at a density of 1E+4 cells/cm² in attachment factor coated 24-well cell culture plates. This density is sufficient for confluence at 3 days post-seeding. AAV carrying a CAG-GFP payload was added to the cell for 48 hours at MOIs calculated by a cell count at the time of transduction and qPCR-derived viral titer. The transduction volume was the same as the standard culture volume (1 ml per well of a 24-well plate). After transduction, media was replenished and every other day thereafter until final readout at seven days post-transduction.

ELISAs for secreted free-AFLB, ANGPT2, and VEGF-A Levels—Cell lysates for ELISA were prepared in M-PER lysis buffer (#78501, Thermo) supplemented with 1× Halt Protease and Phosphatase Inhibitor Cocktail (78440, Thermo) as per manufacturer's instructions. Cell media and lysate were diluted appropriately for each sample and were used to evaluate secreted analyte levels using the Aflibercept ELISA kit (to measure free AFLB levels) (Cat. #IG-AA115, Eagle Biosciences, Nashua, NH), the Quantikine human VEGF-A ELISA kit (DVE00, R&D Systems) and the Quantikine human ANGPT2 ELISA kit (DANG20, R&D Systems) following the provider's instructions. The optical density (OD) was measured with a Cytation 3 (BioTek, Winooski, VT) photometer at 450 nm (reference at OD 620 nm) within 15 min after pipetting the Stop Solution. Media concentrations were defined based on the generated standard curve.

RT-qPCR of Mature miRNAs and Targets from Transduced Cells—cells were lysed on the plate in RLT and total RNA containing miRNAs was purified using the Qiagen RNeasy kit per manufacturer's instructions (#74104, Qiagen), with the modifications for isolating miRNA suggested in the manufacturers supplemental protocol. Briefly, RLT lysate was filtered through a gDNA elimination column, followed by addition of 1.5 volumes of 100% Ethanol to lysate. After running through RNeasy mini column, Wash step with RW1 was skipped and proceeded directly to washing with buffer RPE. Total cDNA was produced using the Maxima RT with dsDNA kit (#M1681, Thermo-Fisher) as per manufacturer's instructions from 100 ng of total RNA. qPCR was performed using TaqMan Fast Advanced Mastermix (#4444963, Thermo-Fisher) and predesigned Taqman probe sets targeting ANGPT2 (Hs00169867_m1, Thermo), a custom Taqman assay against our human optimized AFLB (AR7DTHZ, Thermo) and RPL32 (Hs07291819_s1, Thermo) as a housekeeping control for normalization. Measured levels of VEGF-C and AFLB were normalized to RPL32 expression and expressed as a function of percent reduction from an untreated or vehicle treated control. miRNA specific cDNA was produced using the TaqMan miRNA RT Kit (#4366596, Thermo) as per manufacturer's instructions using 10 ng total RNA and the custom RT primers provided with the ANG2 custom miRNA Taqman Assay (CTU6249, Thermo) targeting the FL mature miRNA guide sequence: 5-AAUGUUCAUACAAUGAGUAAGC-3′ (SEQ ID NO:72). qPCR was performed using TaqMan Fast Advanced Mastermix (#4444963, Thermo-Fisher) and custom Taqman probe sets targeting ANG2 (CTU6249, Thermo). A standard curve was generated from the custom miRvana miRNA mimic of ANG2 (AKS063L. Thermo) with input ranging from 1e9 to 1e2 copies of the miRNA mimic RT product per reaction. miRNA concentrations were calculated from the generated standard curve.

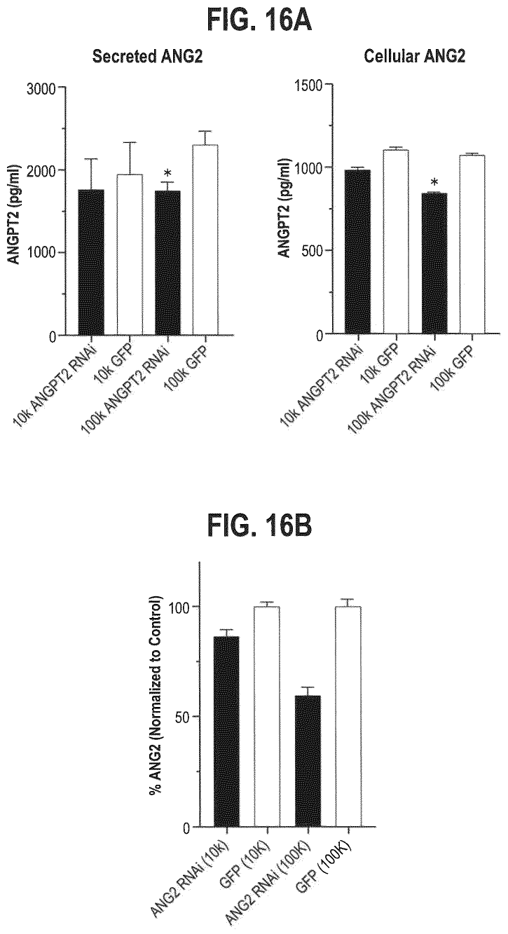

FIG. 16A illustrates results of Ang-2 ELISA showing a reduction of secreted and cellular Ang-2 protein (endogenously made by RMVEC cells) by expression of miRNA targeting Ang-2 in transduced RMVEC cells. A significant decrease in Ang-2 secretion is observed at MOI of 100K; a trend for decreased secretion is also observed at MOI 10 k. A marked decrease in Ang-2 was observed in cellular samples. FIG. 16B illustrates a significant decrease in expression of Ang-2 mRNA at 100 k MOI in transduced RMVEC cells by Ang-2 qPCR

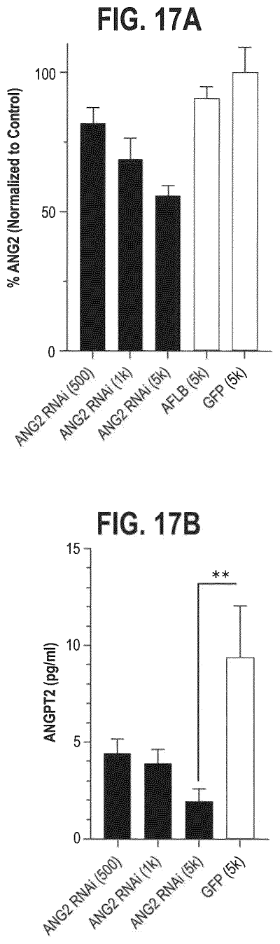

Next, Ang-2 levels were assessed in human RPE cells following transduction with rAAV (the same as described above for RMVEC cells). FIGS. 17A-B illustrate Ang-2 levels at day 8 (post-transduction) in RPE cells by RT-qPCR (FIG. 17A—mRNA levels) and ELISA (FIG. 17B—protein levels). A strong decrease in Ang-2 RNA levels is observed, with KD increasing with increasing MOI. Notably, Ang-2 protein levels in RPE are quite low; however, a trend is observed toward increased KD of Ang-2 protein compared to GFP control-transduced cells at all MOI, trending towards more KD with increased MOI. Significance is observed in matched MOIs.

Next, the effect of including RNAi targeting Ang-2 or VEGF-C on aflibercept expression in dual constructs was assessed in RMVEC cells. Briefly, RMVEC cells were transduced with rAAV comprising a capsid protein of SEQ ID NO:48 and a nucleic acid encoding (i) AFLB only (CAG-AFLB) (ii) encoding AFLB and miRNA targeting Ang-2 (CAG-AFLB-ANG2-RNAi) or (iii) encoding AFLB and miRNA targeting VEGF-C(CAG-AFLB-VEGFC-RNAi).

RMVEC were seeded at a density of 1E+4 cells/cm² in attachment factor coated 24-well cell culture plates. This density is sufficient for confluence at 3 days post-seeding. AAV carrying a CAG-GFP payload was added to the cell for 48 hours at MOIs calculated by a cell count at the time of transduction and qPCR-derived viral titer. The transduction volume was the same as the standard culture volume (1 ml per well of a 24-well plate). After transduction, media was replenished and every other day thereafter until final readout at seven days post-transduction.

RT-qPCR of Mature miRNAs and Targets from Transduced Cells—cells were lysed on the plate in RLT and total RNA containing miRNAs was purified using the Qiagen RNeasy kit per manufacturer's instructions (#74104, Qiagen), with the modifications for isolating miRNA suggested in the manufacturers supplemental protocol. Briefly, RLT lysate was filtered through a gDNA elimination column, followed by addition of 1.5 volumes of 100% Ethanol to lysate. After running through RNeasy mini column, Wash step with RW1 was skipped and proceeded directly to washing with buffer RPE. Total cDNA was produced using the Maxima RT with dsDNA kit (#M1681, Thermo-Fisher) as per manufacturer's instructions from 100 ng of total RNA. qPCR was performed using TaqMan Fast Advanced Mastermix (#4444963, Thermo-Fisher) and predesigned Taqman probe sets targeting ANGPT2 (Hs00169867 ml, Thermo), a custom Taqman assay against our human optimized AFLB (AR7DTHZ, Thermo) and RPL32 (Hs07291819_s1, Thermo) as a housekeeping control for normalization. Measured levels of VEGF-C and AFLB were normalized to RPL32 expression and expressed as a function of percent reduction from an untreated or vehicle treated control. miRNA specific cDNA was produced using the TaqMan miRNA RT Kit (#4366596, Thermo) as per manufacturer's instructions using 10 ng total RNA and the custom RT primers provided with the ANG2 custom miRNA Taqman Assay (CTU6249, Thermo) targeting the FL mature miRNA guide sequence: 5′-AAUGUUCAUACAAUGAGUAAGC-3′ (SEQ ID NO:72). qPCR was performed using TaqMan Fast Advanced Mastermix (#4444963, Thermo-Fisher) and custom Taqman probe sets targeting ANG2 (CTU6249, Thermo). A standard curve was generated from the custom miRvana miRNA mimic of ANG2 (AKS063L, Thermo) with input ranging from 1e9 to 1e2 copies of the miRNA mimic RT product per reaction. miRNA concentrations were calculated from the generated standard curve.

ELISAs for secreted free-AFLB, ANGPT2, and VEGF-A Levels—cell lysates for ELISA were prepared in M-PER lysis buffer (#78501, Thermo) supplemented with 1× Halt Protease and Phosphatase Inhibitor Cocktail (78440, Thermo) as per manufacturer's instructions. Cell media and lysate were diluted appropriately for each sample and were used to evaluate secreted analyte levels using the Aflibercept ELISA kit (to measure free AFLB levels) (Cat. #IG-AA115, Eagle Biosciences, Nashua, NH), the Quantikine human VEGF-A ELISA kit (DVE00, R&D Systems) and the Quantikine human ANGPT2 ELISA kit (DANG20, R&D Systems) following the provider's instructions. The optical density (OD) was measured with a Cytation 3 (BioTek, Winooski, VT) photometer at 450 nm (reference at OD 620 nm) within 15 min after pipetting the Stop Solution. Media concentrations were defined based on the generated standard curve.

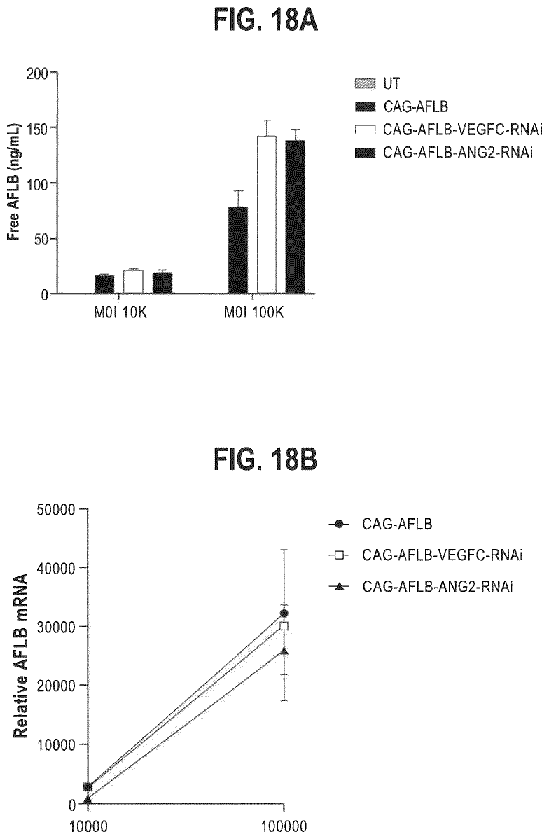

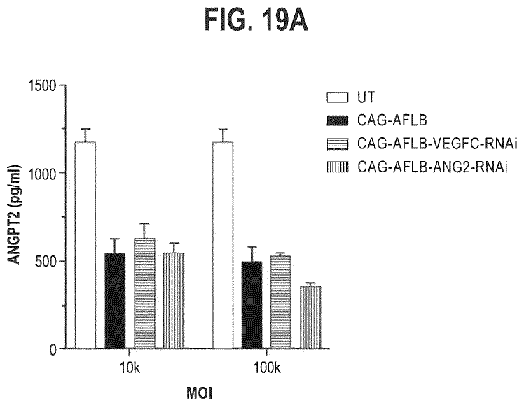

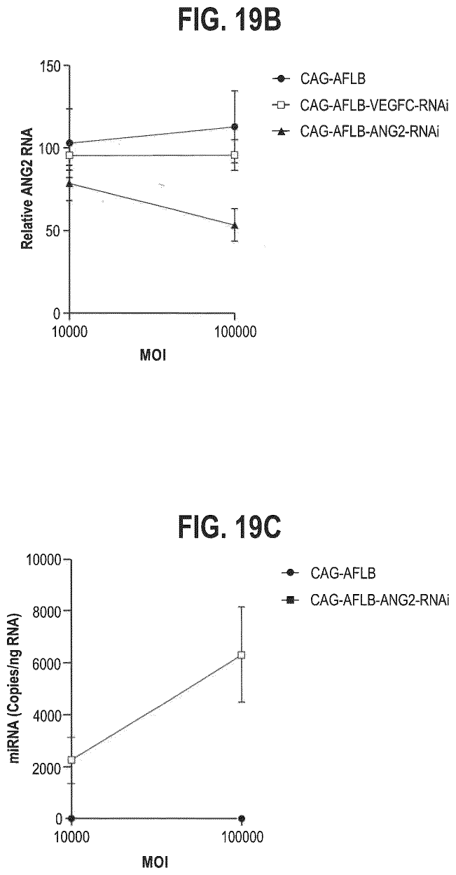

Aflibercept expression was assessed on day 8. FIG. 18A illustrates an increase in free aflibercept levels following transduction with the miRNA constructs compared to transduction with the CAG-AFLB control in both the ANG2 and the VEGF-C RNAi. FIG. 19B illustrates a corresponding dose-dependent increase in aflibercept miRNA.

Ang-2 expression was assessed on day 8. FIG. 19A illustrates a significant decrease in Ang-2 protein levels in all constructs encoding aflibercept, with a further reduction in Ang-2 observed with miRNA targeting Ang-2 compared to other constructs at 100 k MOL. FIG. 19B illustrates a dose response in terms of decrease in Ang-2 transcripts with RNAi targeting Ang-2. Aflibercept alone doesn't appear to affect Ang-2 transcriptionally but may be preventing secretion stimulated by VEGF-A, leading to the decrease observed in supernatants (FIG. 19A). A dose-dependent increase in anti-Ang-2 miRNA (as a function of MOI) is observed (FIG. 19C).

Characterization of VEGF miRNA-Containing Vectors

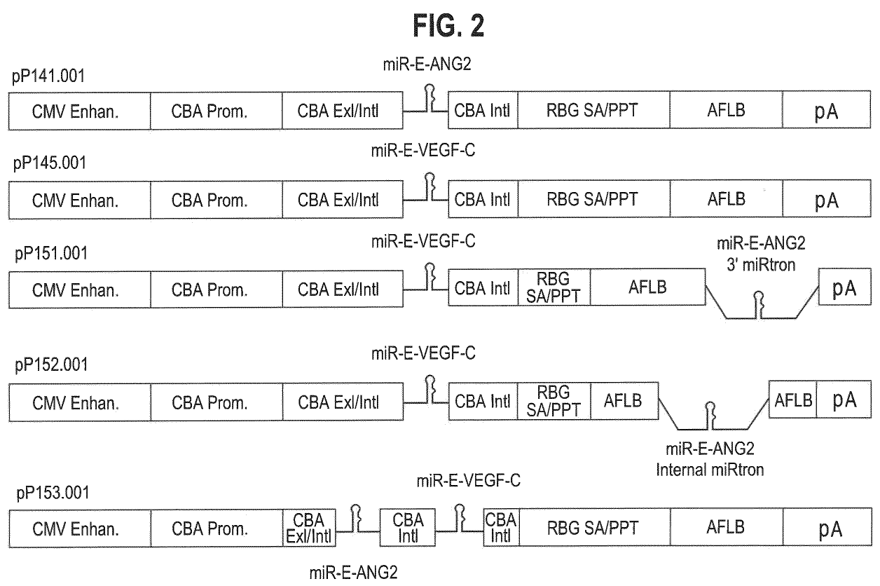

AAV plasmid pP145.001 (pAAV-CACi-miR-E-(VEiFC)-AFLB-SV40) (see FIG. 2) was constructed as follows.

The pAAV-CAG-AFLB-SV40 construct expressing human codon optimized Aflibercept, VEGF-A/B Trap was synthesized. The miR-E-(VEGF-C) miRNA transgene containing the region of the CAG beta-actin intron encoded between the SgrAI and NheI restriction cloning sites was synthesized and cloned into pUC57 by Genscript (Genscript, Picataway, NJ). pUC57 plasmid and the pAAV-CAG-AFLB-SV40-Kan-Stuffer plasmid were cut with various restriction enzymes (New England Biolabs) as indicated, backbone DNA was also treated with recombinant shrimp alkaline phosphatase (rSAP, M0371L, New England Biolabs) during digest to remove free phosphates on cut DNA ends. DNA fragments were added at a 7:1 molar ratio insert:backbone and ligated with Quick Ligase per manufacturer's instructions (#M2200L, New England Biolabs). Ligated plasmid was transformed into NEB Stable bacterial competent cells (#C3040H, New England Biolabs) per manufacturer's instructions and the cells were spread on Kanamycin 50 mg/ml plates (#L1025, Teknova, Hollister, CA) and grown at 30° C.

Miniprep cultures were grown from the resulting colonies (7.1), DNA was prepared with the GeneJET Plasmid Miniprep kit (Cat. #0503, ThermoFisher, Waltham, MA) and restriction digested to identify positive clones. A 50-ml culture in Terrific Broth was grown from one positive clone and DNA was prepared with the Qiagen EndoFree Plasmid Maxi Kit (Cat. #12362, Qiagen, Hilden, Germany).

Maxiprep plasmid DNA (0.5 mg) was digested with various restriction enzymes (New England BioLabs) according to the manufacturer's instructions and analyzed by agarose gel electrophoresis. Sanger DNA sequencing was performed by ELIM using primers.

Several shRNA sequences targeting VEGF-C were evaluated for their ability to reduce expression of VEGF-C in HEK293T cells.

Generation of VEGF-C shRNA Lentiviral Lines—pLKO.1-shVEGFC plasmids were generated by ligation of annealed phosphorylated oligos, corresponding to 5 unique target sequences in human VEGF-C identified from the Broad RNAi consortium (Table 1), into the pLKO.1 vector (Sigma Aldrich, Cat #: SCH001) via EcoRI and AgeI (New England Biolabs) restriction cloning. Plasmid was confirmed by sequencing as with AAV vectors. Maxi prep DNA was generated as with AAV vectors. HEK293T cells were seeded in 6-well plates at 5.0×10≡cells/well in 2.0 ml DMEM/10% FBS media. The next day, 0.5 ug pSF-GFP plasmid DNA, 4.6 uL of MISSION Lentiviral Packaging Mix (Sigma Aldrich, Cat #: SHP001) complexed with 2.7 ul FuGene6 (Cat. #E2691, Promega, Madison, WI) was added to the cells. The next day, the media was replaced with 2 mL of fresh media. On the two subsequent days, media containing lentivirus was collected and replaced on the cells with fresh media. Supernatant was collected and filtered with a 0.45 um syringe filter (Millipore Sigma, Cat #SLHVM33RS), aliquoted and stored at −80° C. Lentivirus was titered using the Lenti-X™ qRT-PCR Titration Kit via qPCR manufacturer's instructions (Takara, Cat #631235). MCF7 cells (#HTB-22, ATCC) were seeded at 5.0×10⁵ cells/well in 6 well plates in 2 ml EMEM/10% FBS media supplemented with 0.01 mg/mL human recombinant insulin (#19278-5ML, Sigma Aldrich). Immediately after plating, cells were transduced with lentivirus at a multiplicity of infection (MOI) of 25 viral genomes per cell. After 48 hrs, media containing lentivirus was removed and replaced with fresh media containing 0.75 ug/mL puromycin (10 mg/ml stock solution, Sigma Aldrich, Cat #: P9620-10 ml). After 72 hrs of selection with puromycin, media containing dead-uninfected cells was removed. Cells expressing shRNA were continually cultured in 0.5 ug/mL puromycin in all experiments to retain shRNA expression. Cells cultured with puromycin were lysed in RLT and total RNA was purified using the Qiagen RNEasy kit per manufacturer's instructions.

qPCR Analysis of MCF7 VEGF-C Knockdown—cells cultured with puromycin were lysed on the plate in RLT and total RNA was purified using the Qiagen RNeasy kit per manufacturer's instructions (#74104, Qiagen). Total cDNA was produced using the Maxima RT with dsDNA kit (#M1681, Thermo-Fisher) as per manufacturer's instructions from 5 ug of total RNA. qPCR was performed using TaqMan Fast Advanced Mastermix (#4444963, Thermo-Fisher) and predesigned Taqman probe sets targeting VEGF-C(Hs01099203_m1, Thermo) and RPL32 (Hs07291819_s1, Thermo) as a housekeeping control for normalization. Measured levels of VEGF-C were normalized to RPL32 expression and expressed as a function of percent reduction from a non-targeting shRNA.

RT-qPCR of Mature miRNAs and Targets from Transduced Cells—cells were lysed on the plate in RLT and total RNA containing miRNAs was purified using the Qiagen RNeasy kit per manufacturer's instructions (#74104, Qiagen), with the modifications for isolating miRNA suggested in the manufacturers supplemental protocol. Briefly, RLT lysate was filtered through a gDNA elimination column, followed by addition of 1.5 volumes of 100% Ethanol to lysate. After running through RNeasy mini column, Wash step with RW1 was skipped and proceeded directly to washing with buffer RPE. Total cDNA was produced using the Maxima RT with dsDNA kit (#M1681, Thermo-Fisher) as per manufacturer's instructions from 100 ng of total RNA. qPCR was performed using TaqMan Fast Advanced Mastermix (#4444963, Thermo-Fisher) and predesigned Taqman probe sets targeting VEGF-C(Hs01099203 ml, Thermo), a custom Taqman assay against our human optimized AFLB (AR7DTHZ, Thermo) and RPL32 (Hs07291819_s1, Thermo) as a housekeeping control for normalization. Measured levels of VEGF-C and AFLB were normalized to RPL32 expression and expressed as a function of percent reduction from an untreated or vehicle treated control. miRNA specific cDNA was produced using the TaqMan miRNA RT Kit (#4366596, Thermo) as per manufacturer's instructions using 10 ng total RNA and the custom RT primers provided with the VEGF-C custom miRNA Taqman Assay (CTTZ9KC, Thermo) targeting the FL mature miRNA guide sequence: 5′-AAUAACGUCUUGCUGAGGUAGC-3′ (SEQ ID NO:73). qPCR was performed using TaqMan Fast Advanced Mastermix (#4444963, Thermo-Fisher) and custom Taqman probe sets targeting VEGF-C(CTTZ9KC, Thermo). A standard curve was generated from the custom miRvana miRNA mimic of VEGF-C(AKT949T, Thermo) with input ranging from 1e9 to 1e2 copies of the miRNA mimic RT product per reaction. miRNA concentrations were calculated from the generated standard curve.

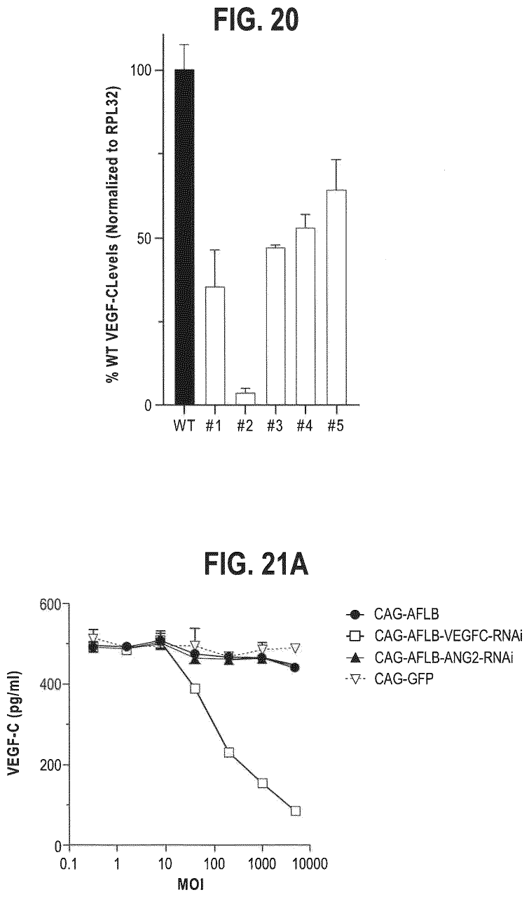

FIG. 20 illustrates the percentage of VEGF-C RNA levels (normalized to RPL32) in HEK293T cells following transduction with the shRNAs. Potent KD of VEGF-C in all constructs was observed, with shRNA #2 resulting in >90% knockdown of VEGF-C. shRNA #2 was selected for inclusion into AAV plasmid constructs encoding aflibercept and miRNA targeting VEGF-C. VEGF-C shRNAs #1-5 comprise the following sense and antisense strand sequences:

| shRNA | | |
|---|---|---|
| Sequence | | |
| # | Sense Strand Sequence | Antisense Strand Sequence |
| 1 | CGCGACAAACACCTTCTTTAA | TTAAAGAAGGTGTTTGTCGCG |
| | (SEQ ID NO: 16) | (SEQ ID NO: 17) |
| 2 | CTACCTCAGCAAGACGTTATT | AATAACGTCTTGCTGAGGTAG |
| | (SEQ ID NO: 19) | (SEQ ID NO: 20) |
| 3 | ACCAATTACATGTGGAATAAT | ATTATTCCACATGTAATTGG |
| | (SEQ ID NO: 22) | (SEQ ID NO: 23) |
| 4 | ATGACCAAACAGCCAAGATTT | AAATCTTGGCTGTTTGGTCA |
| | (SEQ ID NO: 25) | (SEQ ID NO: 26) |
| 5 | GTCGTTGTGTCCCTTCATATT | AATATGAAGGGACACAACGAC |
| | (SEQ ID NO: 28) | (SEQ ID NO: 29) |

Next, human RPE cells were transduced with rAAV comprising a capsid protein of SEQ ID NO:48 and a nucleic acid encoding miRNA comprising the sense and antisense strands of shRNA #2 (the sense and antisense strands of shRNA #2 were embedded within mir-E and the miRNA was placed within the hybrid intron of the CAG promoter). rAAV comprising a capsid protein of SEQ ID NO:48 and a nucleic acid encoding GFP under the control of CAG promoter was used as a control.

RPE Transduction—human stem cell derived retinal pigment epithelial cells (RPE) were differentiated from embryonic stem cells (ESI-017) following published protocols (Buchholz D 2013, Leach L 2015). RPE cells were grown on Matrigel (Corning) for 30 days in XVIVO-10 media (Lonza), in a 96 well plate format. Prior to transduction, three wells were harvested and counted for an accurate calculation of multiplicity of infection (MOI). Virus was added to the cells for 48 hours in XVIVO10 media based on each viral titer in a total volume of 100 μL per well. Media was collected on day 3, 7, 11, 15 and 19 and replaced with 200 μL of media per well. Media samples were stored at 4° C. until processed.

ELISAs for secreted free-AFLB, ANGPT2, and VEGF-A Levels—cell lysates for ELISA were prepared in M-PER lysis buffer (#78501, Thermo) supplemented with 1× Halt Protease and Phosphatase Inhibitor Cocktail (78440, Thermo) as per manufacturer's instructions. Cell media and lysate were diluted appropriately for each sample and were used to evaluate secreted analyte levels using the Aflibercept ELISA kit (to measure free AFLB levels) (Cat. #IG-AA115, Eagle Biosciences, Nashua, NH), the Quantikine human VEGF-A ELISA kit (DVE00, R&D Systems) and the Quantikine human ANGPT2 ELISA kit (DANG20, R&D Systems) following the provider's instructions. The optical density (OD) was measured with a Cytation 3 (BioTek, Winooski, VT) photometer at 450 nm (reference at OD 620 nm) within 15 min after pipetting the Stop Solution. Media concentrations were defined based on the generated standard curve.

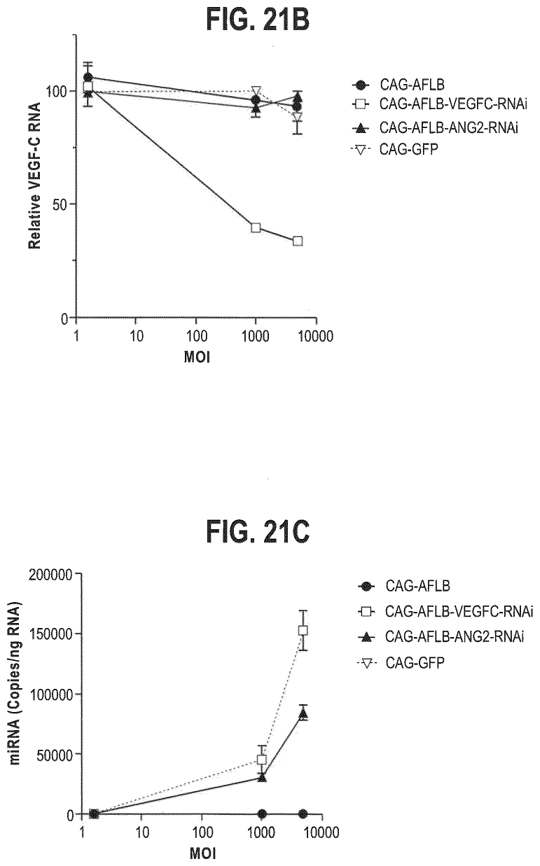

FIG. 21A illustrates VEGF-C levels (by ELISA) in RPE cells following transduction with rAAV carrying the indicated constructs. A dose-dependent decrease in VEGF-C in supernatants of RPE cells (which make VEGF-C endogenously) is observed that is specific to the VEGF-C miRNA construct. A slight decrease in VEGF-C in other constructs is observed at the highest MOI. The miRNA targeting VEGF-C is functional.

FIG. 21B illustrates a corresponding dose-dependent decrease (MOIs of 1.6, 1000 and 5000) in VEGF-C mRNA that is specific to the VEGF-C construct.

FIG. 21C illustrates a dose-dependent increase in expression of miRNA targeting VEGF-C in RPE cells.

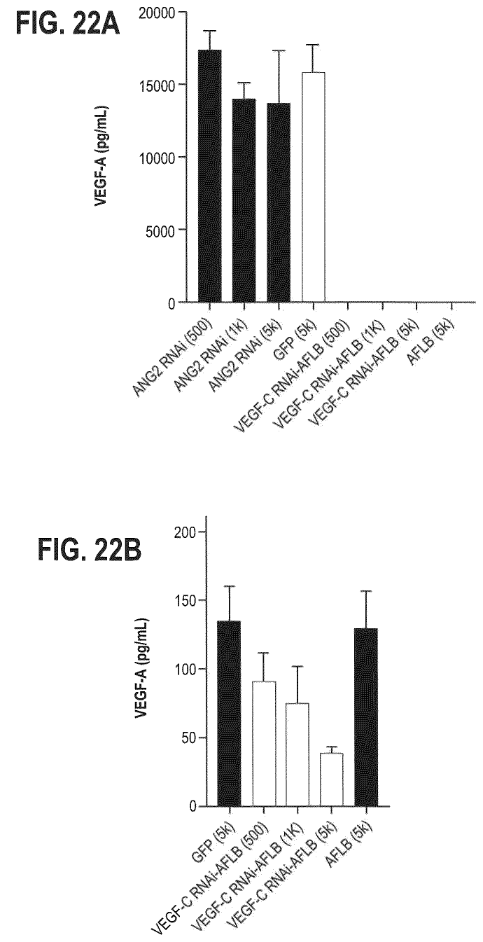

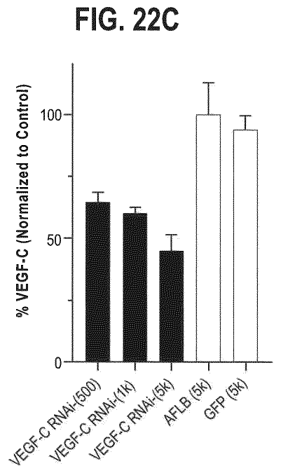

The ability of the dual constructs (encoding aflibercept+miRNA targeting VEGF-C or Ang-2) to neutralize VEGF-A in RPE cells was assessed at day 8 post-transduction with rAAV vectors comprising a capsid protein of SEQ ID NO:48 and nucleic acid comprising the dual construct (“VEGF-C RNAi-AFLB”, “Ang-2 RNAi-AFLB” “AFLB”). FIG. 22A illustrates that all constructs expressing AFLB are able to completely neutralize endogenous VEGF-A at all MOI tested. Notably, constructs not expressing AFLB (“GFP”, “Ang2 RNAi”) do not have any significant effect on VEGF-A levels in the media. FIGS. 21B-C illustrate that VEGF-C protein (FIG. 22B; ELISA) and mRNA levels (FIG. 22C; RT-qPCR) are reduced in dose dependent manner in RPE levels following transduction with rAAV encoding miRNA targeting VEGF-C.

### Example 4—Dual RNAi Vectors

A dual RNAi approach (targeting VEGF-C and Ang-2) was investigated. Representative embodiments included pP151.001, pP152.001 and pP153.001 (see FIG. 2).

pP151 comprises an miRNA targeting Ang-2 placed within an artificial intron located in the 3′ UTR of the aflibercept coding region and an miRNA targeting VEGF-C placed within the hybrid intron of the CAG promoter. pP152 comprises an miRNA targeting Ang-2 placed within an artificial intron located within the aflibercept coding region and an miRNA targeting VEGF-C placed within the hybrid intron of the CAG promoter. pP153 comprises miRNA targeting Ang-2 and miRNA targeting VEGF-C, each placed at different locations within the hybrid intron of the CAG promoter.

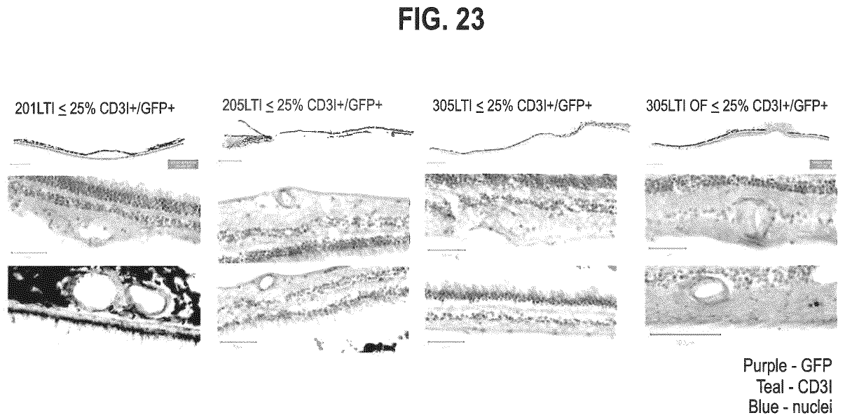

rAAV comprising a capsid comprising SEQ ID NO:48 have been shown to transduce CD31+ endothelial cells following intravitreal administration to non-human primates (1×10″ vg/eye) with a transduction efficiency of <25% of total CD31+ cells (see FIG. 23A—rAAV comprised nucleic acid encoding GFP reporter gene).

### Example 5—NHP Model of Angiogenesis

A pilot pharmacology study was performed in non-human primates (NHPs) to (i) assess acute ocular safety (ii) measure expression of aflibercept and intracellular miR targeting VEGF-C and (iii) confirm the dominant miRNA species in vivo following intravitreal administration of an rAAV comprising a capsid protein of SEQ ID NO:48 and a nucleic acid comprising nucleotide sequence encoding aflibercept and miR targeting VEGF-C. The nucleic acid comprises sense and antisense strands corresponding to SEQ ID Nos: 19 and 20 and the full construct corresponds to SEQ ID NO:69.

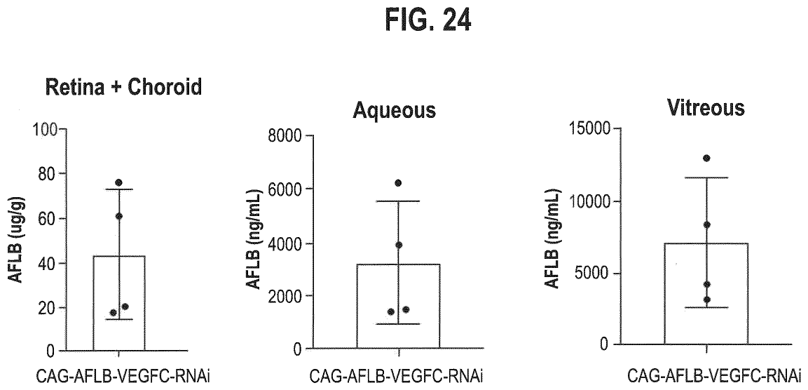

As shown in FIG. 24, aqueous and vitreous aflibercept (AFLB) levels were well within/above the range reported for efficacy. Robust retinal AFLB levels were detected. There was no evidence of uveitis or retinal abnormalities during the study.

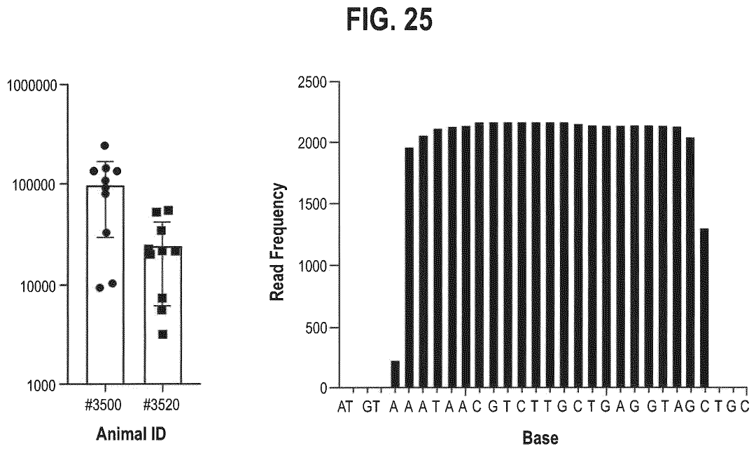

As shown in FIG. 25, a high number of miRNA copies were detected across all retinas of all eyes of NHPs in the study. Inter-animal expression levels parallel what is observed with AFLB ELISA. MiSeq data confirms full-length 22-bp targeting VEGF-C as predominant miRNA species as shown in transfected HEK293T and transduced RPE cells.

Next, a proof-of-concept study was initiated to investigate the efficacy of the rAAV in an NHP model of angiogenesis. See e.g., Goody et al., Experimental Eye Research, 92(6):464-472 (2011). Briefly, African Green NHPs, n=7 per group, were intravitreally administered the rAAV (comprising a capsid protein of SEQ ID NO:48 and a nucleic acid comprising nucleotide sequence encoding aflibercept and miR targeting VEGF-C) or vehicle at three doses (bilaterally at 1×10¹¹ vg/eye, 3×10¹² vg/eye or 1×10¹² vg/eye). Steroids (40 mg methylprednisolone IM weekly starting on D-1 and 2 mg triamcinolone acetonide sub-tenon post-injection) were discontinued after 4 weeks (post-administration). Laser was administered 42 days after dosing to induce choroidal neovascularization (CNV) and lesions were scored 2 and 4 weeks after CNV laser.

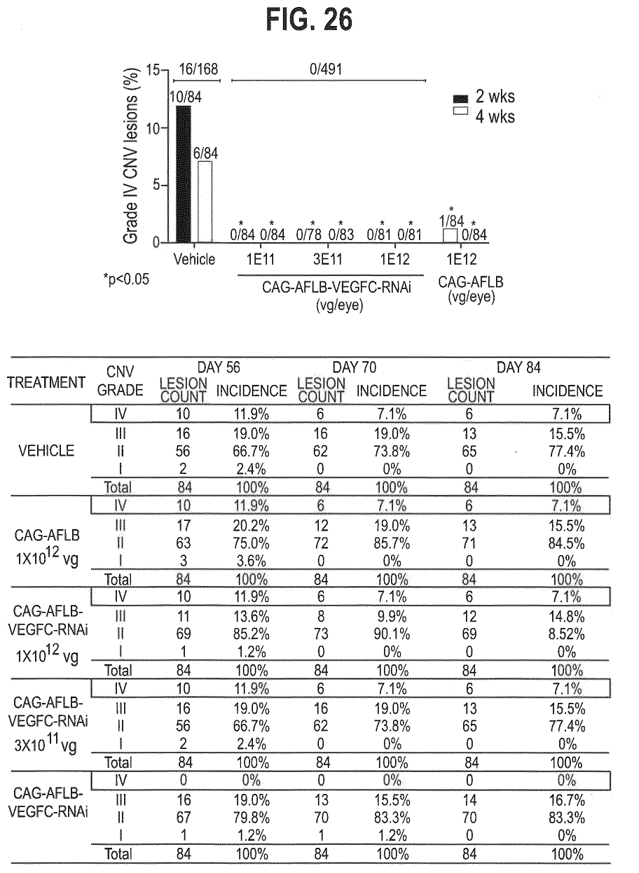

As illustrated in FIG. 26, evaluation of Grade IV lesion incidence revealed that treatment with all doses of the rAAV significantly blocked CNV as demonstrated by complete absence of clinically relevant Grade IV lesions in all treated groups (i.e., at all tested doses of the rAAV). See FIG. 26, compared to vehicle control. No dose-response was observed suggesting complete efficacy of the rAAV at all doses administered.

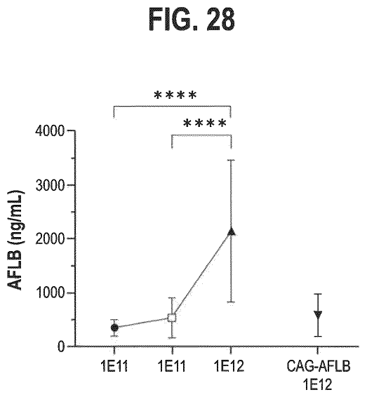

Aqueous humor samples collected from the NHPs at 21 days after intravitreal administration of an rAAV comprising a capsid protein of SEQ ID NO:48 and a nucleic acid encoding aflibercept and miR targeting VEGF-C(comprising the sense and antisense strands corresponding to SEQ ID Nos: 19 and 20 and the full construct corresponding to SEQ ID NO:69), hereinafter referred to as rAAV SEQ ID NO. 48 CAG-AFLB-VEGFC-RNAi, were analyzed for aflibercept protein expression. As shown in FIG. 28, aqueous levels of aflibercept are dose dependent. Additionally, at the 1×10¹² vg/eye dose, aflibercept expression from the eyes dosed with the rAAV SEQ ID NO. 48 CAG-AFLB-VEGFC-RNAi was not inferior to rAAV comprising the capsid protein of SEQ ID NO:48 and a nucleic acid encoding aflibercept only, hereinafter referred to as rAAV SEQ ID NO. 48 CAG-AFLB.

Intraocular inflammation in the NHPs was examined with slit lamp biomicroscopy at designated time points. Scoring was applied to qualitative clinical ophthalmic findings using a nonhuman primate ophthalmic exam scoring system with a summary score derived from exam components. At designated time points, intraocular pressure (IOP) measurements were collected using a TonoVet (iCare, Finland) tonometer set to the dog (d) calibration setting. Three measures were taken from each eye at each time point and the mean IOP defined.

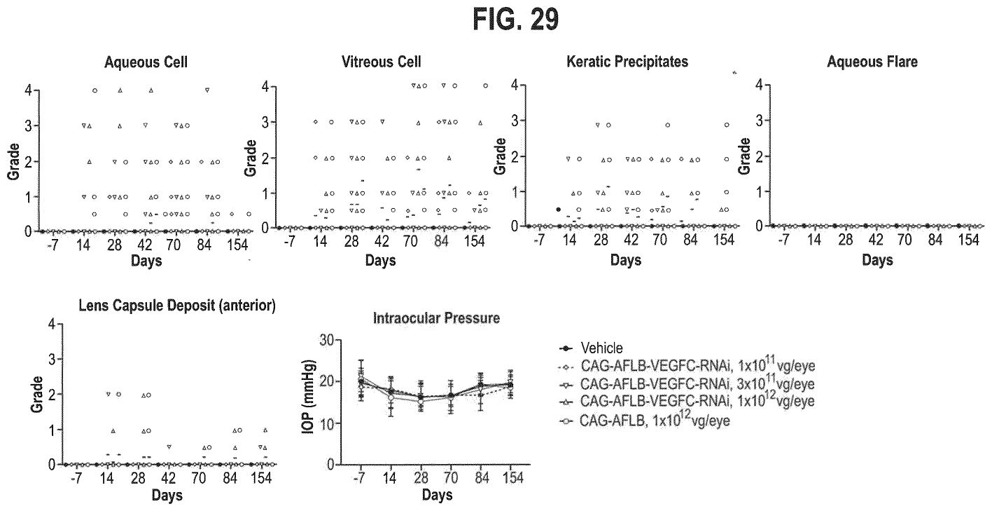

In comparison to vehicle-treated eyes that exhibited consistently low integrated clinical score, the eyes receiving rAAV SEQ ID NO. 48 CAG-AFLB at 1×10¹¹ vg/eye or rAAV SEQ ID NO. 48 CAG-AFLB-VEGFC-RNAi at 1×10¹² vg/eye exhibited mild to moderate intraocular inflammation, peaking around 28 days post IVT injection (see FIG. 29).

At Week 22, there was no or only mild intraocular inflammation in eyes treated with vehicle, rAAV SEQ ID NO. 48 CAG-AFLB-VEGFC-RNAi at 1×10¹¹ vg/eye or 3×10¹¹ vg/eye, while half or more of the eyes treated with rAAV SEQ ID NO.48 CAG-AFLB at 1×10¹¹ vg/eye or rAAV SEQ ID NO. 48 CAG-AFLB-VEGFC-RNAi at 1×10¹² vg/eye exhibited mild to moderate intraocular inflammation. See FIG. 29. The inflammatory responses mainly included mild aqueous cells, keratic precipitates, vitreous cell findings, and deposits on the anterior capsular membrane of the lens. Intraocular pressure (TOP) remained normal in all groups.

Retinal volume and central retinal thickness were assessed in the NHPs. Briefly, at designated time points, Optical Coherence Tomography (OCT) was performed using a Heidelberg Spectralis OCT Plus with eye tracking and HEYEX image capture and analysis software. An overall volume scan of encompassing the posterior retina was performed. At examinations prior to laser, the retinal thickness map and cross-sectional display image were obtained.

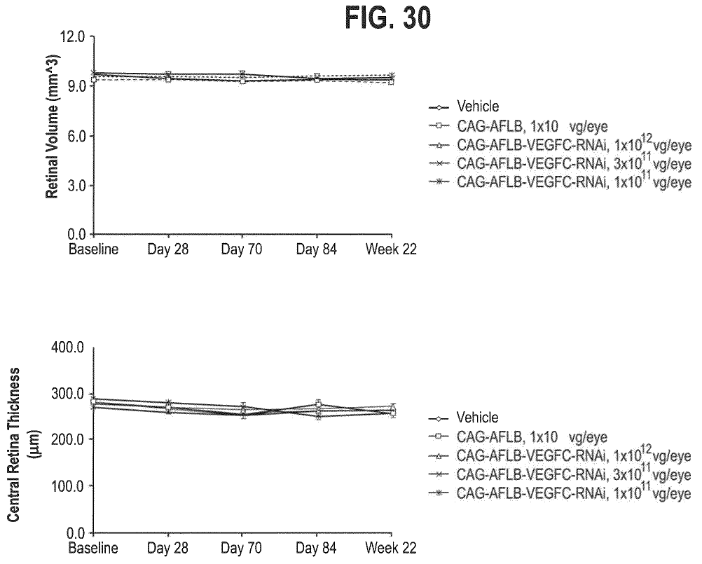

OCT derived retinal volume and retinal thickness exhibited a stable comprehensive retinal thickness from baseline to 22 weeks, indicating no retinal edema or degeneration-related thinning occurred during the observation period after any treatment. Mean values of the sum of retinal volume and average central retinal thickness within an applied ETDRS grid remained stable throughout the study (see FIG. 30).

Full Field Electroretinography (ffERG) was conducted at Day 84 and Week 22 to compare the change of retinal function in the NHPs. Briefly, a minimum 25-minute period of dark-adaptation preceded scotopic ffERG recording. Dark adaptation was achieved by retaining the monkey under sedation in a transfer cage situated in a dark room accessed with a scotopic red light. Pupils were dilated with phenylephrine (10%), augmented with cyclopentolate (1%), at the beginning of dark adaptation and potentially again before stimulus exposure to ensure that animals had maximum pupil dilation at the point of stimulus induction.

A minimum 10-minute period of light-adaptation preceded photopic ffE RG recording with the eyes kept open and DTL electrodes kept in place. Pupils were dilated with phenylephrine (10%), augmented with cyclopentolate (1%), at the beginning of light adaptation and potentially again before stimulus exposure to ensure that monkeys had maximum pupil dilation at the point of stimulus induction.

The following procedures were performed using the Veris platform to the ISCEV standards for a toxicology study and included the following stimulus exposures:
Scotopic 0.16 cd-s m2 stimulus (rod-driven response of on bipolar cells measured, b wave)
Scotopic 2.51 cd-s m2 stimulus (rod and cone-driven response of both photoreceptors, a wave, and on bipolar cells, b wave)
Photopic 2.51 cd-s m2 stimulus (cone driven response of both photoreceptors, a wave, and on and off bipolar cells, b wave)
Photopic 30 Hz flicker stimulus at 2.51 cd-s m2 stimulus (cone driven response)

NHPs underwent scotopic exams before photopic exams and always underwent stimulus exposure order of increasing stimulus strength for a given adaptation. Single stimulus exposures always preceded flicker stimulus exposure to avoid bleaching and impacting retinal adaptation.

To validate consistency as well as establish the range of variability inherent in ffERGs, each stimulus at each time point was captured by two independent runs. Each run was a composite of 3 separate, sequential stimulus inductions.

Data recording followed the format guided by the ISCEV standards.
Stimulus induction was denoted by a marker
The time integrated luminance of not only the stimulus, but also the background was recorded in absolute values
Time and date of stimulus induction
Pupil diameter
Type and position of corneal electrodes

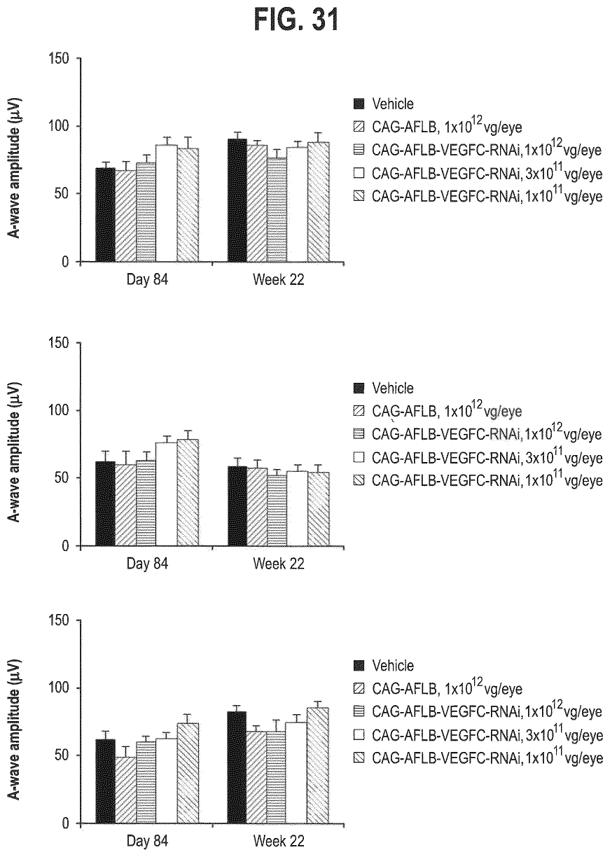

No statistically significant difference of scotopic A wave, scotopic B wave and photopic flicker was observed between treatment groups at the same time point or between different time points of the same treatment group (all p>0.05, Two-way ANOVA followed by Tukey-Krammer HSD). The mean amplitude of scotopic A wave, scotopic B wave and photopic flicker are presented in FIG. 31.

CONCLUSION—rAAV comprising a capsid protein of SEQ ID NO:48 and a nucleic acid encoding aflibercept+miR targeting VEGF-C completely abolished grade IV lesion development compared to vehicle-treated controls and significantly attenuated CNV development, supporting the safety and efficacy of the rAAV in the treatment of a variety of diseases associated with ocular angiogenesis such as wet AMD.

### Example 6—HUVEC Proliferation & Migration Assays

In vitro angiogenesis assays were performed to assess effects of plasmids encoding (i) aflibercept+miRNA targeting VEGF-C(CAG-AFLB-VEGFC-RNAi) (ii) aflibercept only (CAG-AFLB) or (iii) GFP (CAG-GFP) on proliferation and migration of human umbilical vein endothelial cells (HUVECs) following electroporation. Briefly, HUVECs were lifted using 0.05% Trypsin EDTA and electroporated according to Thermo Fisher Neon Electroporator kit instructions. Two million cells per condition were resuspended in R buffer with the appropriate amount of plasmid. One microgram of total DNA was transfected per condition (CAG-AFLB-VEGFC-RNAi, CAG-AFLB, or CAG-GFP). Equimolar concentrations of CAG-AFLB-VEGFC-RNAi plasmid and CAG-AFLB plasmids were transfected. Because the length of CAG-AFLB plasmid (6,660 bp; 1.4×10¹¹ copies/μg) was less that CAG-AFLB-VEGFC-RNAi plasmid (10,711 bp: 8.6×10¹⁰ copies/μg), extra CAG-GFP plasmid was added to the CAG-AFLB condition to equalize the total DNA. A mock transfection “Shock” was also performed as a control. Cells were electroporated by a single pulse at 1350 V for 30 milliseconds. Media was changed four hours post-electroporation to remove residual R buffer. Following electroporation cells were plated for proliferation or migration assays.

HUVEC Cell Counts for Proliferation Assay

Four days post-electroporation, cells were lifted with 0.05% Trypsin-EDTA. Trypsin was quenched with an equal volume of complete media. Cells were centrifuged at 400×g for five minutes and resuspended in 50 μl complete media. Cell suspension was counted using a BD countess cell counter. Six replicates per condition were counted. The total experiment was run three distinct times.

HUVEC Cell Counts for Migration Assay

Four days post-electroporation, cells were lifted with 0.05% Trypsin-EDTA. Trypsin was quenched with an equal volume of complete media. Cells were centrifuged at 400×g for five minutes and counted. 25,000 cells were seeded in starvation EGM-2 medium (without VEGF) into the upper compartment of an 8 μm pore transwell insert coated with 0.1% gelatin, according to Nareshkumar et al. Scientific reports. 8.1 (2018): 1-16. The bottom compartment contained complete EGM-2 media, creating a growth factor gradient. Four hours post-seeding, cultures were fixed and washed with PBS. Nuclei were counterstained with DAPI for 5 minutes at room temperature. The upper compartment was then scraped thoroughly with a rubber scraper. Images were taken at 50× magnification using a Zeiss AxioVert.A1 fluorescent microscope. Four images per insert were taken in a non-biased grid pattern, three replicates per transfection condition. The total experiment was run three distinct times. Quantification of DAPI was done using FIJI software. Schindelin, et al. Nature methods. 9.7 (2012): 676-682. Briefly, a threshold was applied to each image and converted to binary mask. DAPI points were then quantified using the “analyze particles” function. Thresholds were the same within each experiment, but different between experimental replicates because of variability in DAPI staining intensity.

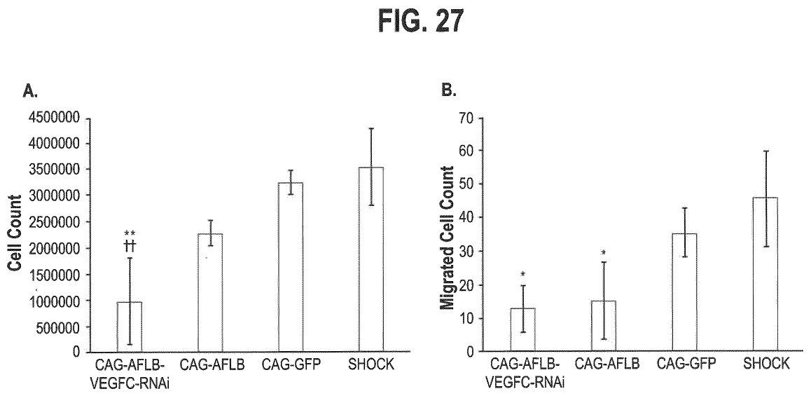

As shown in FIG. 27, HUVEC cells transfected with plasmid DNA comprising a nucleic acid encoding aflibercept and miR targeting VEGF-C led to a decrease in number of cells present in the culture system compared to plasmid containing GFP under the control of CAG promoter or Shock conditions controls (FIG. 27A). The nucleic acid encoding aflibercept and miR targeting VEGF-C comprises sense and antisense strands corresponding to SEQ ID Nos: 19 and 20 and the full construct corresponds to SEQ ID NO:69. In addition, fewer cells migrated through the transwell membrane after transfection with plasmid DNA comprising the nucleic acid encoding aflibercept and miR targeting VEGF-C compared to the Shock condition (FIG. 27B). Importantly, in both assays, the plasmid containing the nucleic acid encoding aflibercept and miR targeting VEGF-C was not inferior to cells transfected with plasmid containing aflibercept under control of the CAG promoter only. These data demonstrate robust inhibition of endothelial cell proliferation and migration by nucleic acid encoding aflibercept and miR targeting VEGF-C in HUVEC cells.

While the materials and methods of this invention have been described in terms of preferred embodiments, it will be apparent to those of skill in the art that variations may be applied to the method described herein without departing from the concept, spirit and scope of the invention. All such similar substitutes and modifications apparent to those skilled in the art are deemed to be within the spirit, scope and concept of the invention.

1. A synthetic ribonucleic acid (RNA) molecule comprising a sense strand comprising at least 90% sequence identity with a sequence selected from SEQ ID Nos: 19, 16, 22, 25, 28, 1, 4, 7, 10, 13, 31, 34, 37, 40 and 43 and an antisense strand comprising at least 90% identity with a sequence selected from SEQ ID Nos: 20, 17, 23, 26, 29, 2, 5, 8, 11, 14, 32, 35, 38, 41 and 44, respectively.

2. The synthetic RNA molecule according to claim 1, wherein the RNA is a small interfering RNA (siRNA), a small hairpin RNA (shRNA) or an artificial microRNA (miRNA).

3. The synthetic RNA molecule according to claim 1, wherein the RNA is an shRNA comprising a loop comprising the sequence CTCGAG or a sequence at least 90% identical thereto.

4. The synthetic RNA molecule according to claim 1, wherein the RNA is an artificial miRNA comprising RNA comprising at least 90% sequence identity with the sequence set forth as SEQ TD NO:46 or 47, wherein (X)ₙ comprises a sense sequence comprising at least 90% sequence identity with a sequence selected from SEQ ID Nos: 19, 16, 22, 25, 28, 1, 4, 7, 10, 13, 31, 34, 37, 40 and 43 and (Y)ₙ comprises an antisense sequence comprising at least 90% sequence identity with a sequence selected from SEQ ID Nos: 20, 17, 23, 26, 29, 2, 5, 8, 11, 14, 32, 35, 38, 41 and 44, respectively.

5. The synthetic RNA according to claim 4, wherein (X)ₙ comprises a sense sequence comprising the nucleotide sequence set forth as SEQ ID No: 19 and (Y)ₙ comprises an antisense sequence comprising the nucleotide sequence set forth as SEQ ID No: 20.

6. A nucleic acid comprising a nucleotide sequence encoding at least one synthetic RNA molecule according to claim 1.

7. The nucleic acid of claim 6 wherein the nucleic acid comprises (i) a nucleotide sequence encoding an artificial miRNA comprising RNA comprising at least 90% sequence identity with the sequence set forth as SEQ ID NO:46 or 47, wherein (X)ₙ comprises a sense sequence comprising the nucleotide sequence set forth as SEQ ID No: 19 and (Y)ₙ comprises an antisense sequence comprising the nucleotide sequence set forth as SEQ ID No: 20 and further comprises (ii) a nucleotide sequence encoding a first anti-angiogenic polypeptide that inhibits the activity of VEGF-A, wherein the nucleotide sequence of (i) and (ii) are operably linked to an expression control sequence.

8. The nucleic acid according to claim 7, wherein the first anti-angiogenic polypeptide is aflibercept.

9. The nucleic acid according to claim 8, wherein the nucleotide sequence encoding the first anti-angiogenic polypeptide and the nucleotide sequence encoding one or more synthetic RNA molecules are under the control of the same expression control sequence.

10. The nucleic acid according to claim 9, wherein the expression control sequence comprises a ubiquitous promoter.

11. The nucleic acid according to claim 10, wherein the promoter is a CAG or CBA promoter.

12. The nucleic acid according to claim 8, wherein the nucleic acid comprises the nucleotide sequence set forth in any one of SEQ ID Nos: 69, 68, and 70 or a sequence at least 95% identical thereto.

13. A composition comprising the nucleic acid according to claim 8.

14. The composition according to claim 13, which is a pharmaceutical composition further comprising a pharmaceutically acceptable carrier.

15. A vector comprising the nucleic acid according to claim 8.

16. The vector according to claim 15 wherein the vector is a recombinant adeno-associated virus (rAAV) vector.

17. The vector according to claim 16, wherein the rAAV has a capsid comprising a capsid protein comprising a heterologous peptide insertion with a length of 7, 8, 9, 10 or 11 amino acids covalently inserted in the GH-loop of the capsid protein relative to a corresponding parental AAV capsid protein, wherein the peptide insertion comprises the amino acid sequence ISDQTKH (SEQ ID NO:74).

18. The vector according to claim 17, wherein the insertion peptide is LAISDQTKHA (SEQ ID NO:49).

19. The vector according to claim 17, wherein the insertion site is located between amino acids corresponding to amino acids 587 and 588 of VP1 of AAV2 or between amino acids corresponding to amino acids 588 and 589 of VP1 of AAV2 or the corresponding position in the capsid protein of another AAV serotype.

20. The vector according to claim 17, wherein the variant capsid protein comprises a V7081 amino acid substitution relative to VP1 of AAV2 or the corresponding position in the capsid protein of another

21. The vector according to claim 20, wherein the rAAV has a capsid comprising a capsid protein comprising an amino acid sequence at least 90% identical to SEQ ID NO:48.

22. The vector according to claim 21, wherein the rAAV comprises a capsid comprising a capsid protein comprising an amino acid sequence at least 95% identical to SEQ ID NO:48 and a heterologous nucleic acid comprising the sequence set forth as SEQ ID No: 69.

23. A pharmaceutical composition comprising the vector according to claim 16 and a pharmaceutically acceptable carrier.

24. The pharmaceutical composition according to claim 23, wherein the rAAV comprises a capsid protein comprising an amino acid sequence at least 95% identical to SEQ ID NO:48 and a heterologous nucleic acid comprising a nucleotide sequence at least 95% identical to the nucleotide sequence set forth as SEQ ID NO:69.

25. A method for treating an ocular disease associated with VEGF-A in a subject, comprising administering to the subject a pharmaceutical composition according to claim 24.

26. The method according to claim 25, wherein the pharmaceutical composition is administered to the subject intraocularly.

27. The method according to claim 26, wherein the pharmaceutical composition comprises 1×10⁹ to 1×10¹⁴ vector particles.

28. The method according to claim 25, wherein the disease associated with ocular angiogenesis is selected from the group consisting of wet age-related macular degeneration; macular edema following retinal vein occlusion; retinal neovascularization resulting from retinal vein occlusion; diabetic macular edema, diabetic retinopathy, myopic macular degeneration, branch retinal vein occlusion, hemi-retinal vein occlusion, and central retinal vein occlusion; retinopathy of prematurity; idiopathic choroidal neovascularization; myopia macular degeneration and secondary retinal and choroidal neovascularization; retinal telangiectasia; neovascular glaucoma; vitreous hemorrhage; retinal and choroidal neovascularization secondary to retinal diseases, including but not limited to uveitis, trauma, retinal degenerative disorders, genetic retinal and/or choroidal disease, tumors of the eye, corneal and iris neovascularization.

29. The method according to claim 28, wherein the disease associated with VEGF-A is selected from wet age-related macular degeneration; diabetic macular edema; macular edema following retinal vein occlusion; diabetic retinopathy; and myopic choroidal neovascularization.

30. A method for delivering a synthetic RNA to a retinal cell comprising contacting the retinal cell with the synthetic RNA according to claim 1.
